SEQUENCE LISTING (12 sequences)

SEQ ID NO: 1 - DNA, 1635 bp, Populus alba ispS
atggaagcacgtcgctctgcgaactacgaacctaacagctgggactatgattacctgctgtcctccgacacggacgagtccatcgaagtatacaaagacaaagcgaaaaagctggaagccgaagttcgtcgcgagattaataacgaaaaagcagaatttctgaccctgctggaactgattgacaacgtccagcgcctgggcctgggttaccgtttcgagtctgatatccgtggtgcgctggatcgcttcgtttcctccggcggcttcgatgcggtaaccaagacttccctgcacggtacggcactgtctttccgtctgctgcgtcaacacggttttgaggtttctcaggaagcgttcagcggcttcaaagaccaaaacggcaacttcctggagaacctgaaggaagatatcaaagctatcctgagcctgtacgaggccagcttcctggctctggaaggcgaaaacatcctggacgaggcgaaggttttcgcaatctctcatctgaaagaactgtctgaagaaaagatcggtaaagagctggcagaacaggtgaaccatgcactggaactgccactgcatcgccgtactcagcgtctggaagcagtatggtctatcgaggcctaccgtaaaaaggaggacgcgaatcaggttctgctggagctggcaattctggattacaacatgatccagtctgtataccagcgtgatctgcgtgaaacgtcccgttggtggcgtcgtgtgggtctggcgaccaaactgcactttgctcgtgaccgcctgattgagagcttctactgggccgtgggtgtagcattcgaaccgcaatactccgactgccgtaactccgtcgcaaaaatgttttctttcgtaaccattatcgacgatatctacgatgtatacggcaccctggacgaactggagctgtttactgatgcagttgagcgttgggacgtaaacgccatcaacgacctgccggattacatgaaactgtgctttctggctctgtataacactattaacgaaatcgcctacgacaacctgaaagataaaggtgagaacatcctgccgtatctgaccaaagcctgggctgacctgtgcaacgctttcctgcaagaagccaagtggctgtacaacaaatctactccgacctttgacgactacttcggcaacgcatggaaatcctcttctggcccgctgcaactggtgttcgcttacttcgctgtcgtgcagaacattaaaaaggaagagatcgaaaacctgcaaaaataccatgacaccatctctcgtccttcccatatcttccgtctgtgcaatgacctggctagcgcgtctgcggaaattgcgcgtggtgaaaccgcaaatagcgtttcttgttacatgcgcactaaaggtatctccgaagaactggctaccgaaagcgtgatgaatctgatcgatgaaacctggaaaaagatgaacaaggaaaaactgggtggtagcctgttcgcgaaaccgttcgtggaaaccgcgatcaacctggcacgtcaatctcactgcacttatcataacggcgacgcgcatacctctccggatgagctgacccgcaaacgcgttctgtctgtaatcactgaaccgattctgccgtttgaacgctaa

SEQ ID NO: 2 - protein, 329 aa, Artificial Sequence
  Synthetic Construct
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 3 - DNA, 2439 bp, Listeria grayi DSM 20601 mvaE
atggttaaagacattgtaataattgatgccctccgtactcccatcggtaagtaccgcggtcagctctcaaagatgacggcggtggaattgggaaccgcagttacaaaggctctgttcgagaagaacgaccaggtcaaagaccatgtagaacaagtcatttttggcaacgttttacaggcagggaacggccagaatcccgcccgtcagatcgcccttaattctggcctgtccgcagagataccggcttcgactattaaccaggtgtgtggttctggcctgaaagcaataagcatggcgcgccaacagatcctactcggagaagcggaagtaatagtagcaggaggtatcgaatccatgacgaatgcgccgagtattacatattataataaagaagaagacaccctctcaaagcctgttcctacgatgaccttcgatggtctgaccgacgcgtttagcggaaagattatgggtttaacagccgaaaatgttgccgaacagtacggcgtatcacgtgaggcccaggacgcctttgcgtatggatcgcagatgaaagcagcaaaggcccaagaacagggcattttcgcagctgaaatactgcctcttgaaataggggacgaagttattactcaggacgagggggttcgtcaagagaccaccctcgaaaaattaagtctgcttcggaccatttttaaagaagatggtactgttacagcgggcaacgcctcaacgatcaatgatggcgcctcagccgtgatcattgcatcaaaggagtttgctgagacaaaccagattccctaccttgcgatcgtacatgatattacagagataggcattgatccatcaataatgggcattgctcccgtgagtgcgatcaataaactgatcgatcgtaaccaaattagcatggaagaaatcgatctctttgaaattaatgaggcatttgcagcatcctcggtggtagttcaaaaagagttaagcattcccgatgaaaagatcaatattggcggttccggtattgcactaggccatcctcttggcgccacaggagcgcgcattgtaaccaccctagcgcaccagttgaaacgtacacacggacgctatggtattgcctccctgtgcattggcggtggccttggcctagcaatattaatagaagtgcctcaggaagatcagccggttaaaaaattttatcaattggcccgtgaggaccgtctggctagacttcaggagcaagccgtgatcagcccagctacaaaacatgtactggcagaaatgacacttcctgaagatattgccgacaatctgatcgaaaatcaaatatctgaaatggaaatccctcttggtgtggctttgaatctgagggtcaatgataagagttataccatcccactagcaactgaggaaccgagtgtaatcgctgcctgtaataatggtgcaaaaatggcaaaccacctgggcggttttcagtcagaattaaaagatggtttcctgcgtgggcaaattgtacttatgaacgtcaaagaacccgcaactatcgagcatacgatcacggcagagaaagcggcaatttttcgtgccgcagcgcagtcacatccatcgattgtgaaacgaggtgggggtctaaaagagatagtagtgcgtacgttcgatgatgatccgacgttcctgtctattgatctgatagttgatactaaagacgcaatgggcgctaacatcattaacaccattctcgagggtgtagccggctttctgagggaaatccttaccgaagaaattctgttctctattttatctaattacgcaaccgaatcaattgtgaccgccagctgtcgcataccttacgaagcactgagtaaaaaaggtgatggtaaacgaatcgctgaaaaagtggctgctgcatctaaatttgcccagttagatccttatcgagctgcaacccacaacaaaggtattatgaatggtattgaggccgtcgttttggcctcaggaaatgacacacgggcggtcgcggcagccgcacatgcgtatgcttcacgcgatcagcactatcggggcttaagccagtggcaggttgcagaaggcgcgttacacggggagatcagtctaccacttgcactcggcagcgttggcggtgcaattgaggtcttgcctaaagcgaaggcggcattcgaaatcatggggatcacagaggcgaaggagctggcagaagtcacagctgcggtagggctggcgcaaaacctggcggcgttaagagcgcttgttagtgaaggaatacagcaaggtcacatgtcgctccaggctcgctctcttgcattatcggtaggtgctacaggcaaggaagttgaaatcctggccgaaaaattacagggctctcgtatgaatcaggcgaacgctcagaccatactcgcagagatcagatcgcaaaaagttgaattgtga

SEQ ID NO: 4 - DNA, 1158 bp, Enterococcus faecium mvaE
atgaccatgaacgttggaatcgataaaatgtcattctttgttccaccttactttgtggacatgactgatctggcagtagcacgggatgtcgatcccaataagtttctgattggtattggccaggaccagatggcagttaatccgaaaacgcaggatattgtgacatttgccacaaatgctgccaaaaacatactgtcagctgaggaccttgataaaattgatatggtcatagtcggcaccgagagtggaatcgatgaatccaaagcgagtgccgtagtgcttcacaggttgctcggtatccagaagtttgctcgctcctttgaaatcaaagaagcctgttatgggggtaccgcggctttacagttcgctgtaaaccacattaggaatcatcctgaatcaaaggttcttgtagttgcatcagatatcgcgaaatacggcctggcttctggaggtgaaccaacgcaaggtgcaggcgctgtggctatgctcgtctcaactgaccctaagatcattgctttcaacgacgatagcctcgcgcttacacaagatatctatgacttctggcgaccagttggacatgactatcctatggtcgacgggcctcttagtacagagacctacatccagtcatttcagaccgtatggcaggaatacacaaaacggtcgcagcatgcactggcagactttgctgcccttagctttcatatcccgtatactaaaatgggcaaaaaggcgctgcttgcaatccttgaaggcgaatcagaggaggctcagaaccgtatactagcaaaatatgaaaagagtatagcctactccagaaaggcgggtaacctgtataccggtagcctgtatctaggacttatttcacttctggaaaatgcagaagaccttaaagctggtgatttaataggcctcttttcttacggttccggtgctgttgcggagtttttctcaggaaggctggttgaggactatcaggaacagctacttaaaacaaaacatgccgaacagctggcccatagaaagcaactgacaatcgaggagtacgaaacgatgttctccgatcgcttggacgtggacaaagacgccgaatacgaagacacattagcttatagcatttcgtcagtccgaaacaccgtacgtgagtacaggagttga

SEQ ID NO: 5 - DNA, 2442 bp, Enterococcus gallinarum EG2 mvaE
atgaaagaagtggttatgattgatgcggctcgcacacccattgggaaatacagaggtagtcttagtccttttacagcggtggagctggggacactggtcacgaaagggctgctggataaaacaaagcttaagaaagacaagatagaccaagtgatattcggcaatgtgcttcaggcaggaaacggacaaaacgttgcaagacaaatagccctgaacagtggcttaccagttgacgtgccggcgatgactattaacgaagtttgcgggtccggaatgaaagcggtgattttagcccgccagttaatacagttaggggaggcagagttggtcattgcagggggtacggagtcaatgtcacaagcacccatgctgaaaccttaccagtcagagaccaacgaatacggagagccgatatcatcaatggttaatgacgggctgacggatgcgttttccaatgctcacatgggtcttactgccgaaaaggtggcgacccagttttcagtgtcgcgcgaggaacaagaccggtacgcattgtccagccaattgaaagcagcgcacgcggttgaagccggggtgttctcagaagagattattccggttaagattagcgacgaggatgtcttgagtgaagacgaggcagtaagaggcaacagcactttggaaaaactgggcaccttgcggacggtgttttctgaagagggcacggttaccgctggcaatgcttcaccgctgaatgacggcgctagtgtcgtgattcttgcatcaaaagaatacgcggaaaacaataatctgccttacctggcgacgataaaggaggttgcggaagttggtatcgatccttctatcatgggtattgccccaataaaggccattcaaaagttaacagatcggtcgggcatgaacctgtccacgattgatctgttcgaaattaatgaagcattcgcggcatctagcattgttgtttctcaagagctgcaattggacgaagaaaaagtgaatatctatggcggggcgatagctttaggccatccaatcggcgcaagcggagcccggatactgacaaccttagcatacggcctcctgcgtgagcaaaagcgttatggtattgcgtcattatgtatcggcggtggtcttggtctggccgtgctgttagaagctaatatggagcagacccacaaagacgttcagaagaaaaagttttaccagcttaccccctccgagcggagatcgcagcttatcgagaagaacgttctgactcaagaaacggcacttattttccaggagcagacgttgtccgaagaactgtccgatcacatgattgagaatcaggtctccgaagtggaaattccaatgggaattgcacaaaattttcagattaatggcaagaaaaaatggattcctatggcgactgaagaaccttcagtaatagcggcagcatcgaacggcgccaaaatctgcgggaacatttgcgcggaaacgcctcagcggcttatgcgcgggcagattgtcctgtctggcaaatcagaatatcaagccgtgataaatgccgtgaatcatcgcaaagaagaactgattctttgcgcaaacgagtcgtacccgagtattgttaaacgcgggggaggtgttcaggatatttctacgcgggagtttatgggttcttttcacgcgtatttatcaatcgactttctggtggacgtcaaggacgcaatgggggcaaacatgatcaactctattctcgaaagcgttgcaaataaactgcgtgaatggttcccggaagaggaaatactgttctccatcctgtcaaacttcgctacggagtccctggcatctgcatgttgcgagattccttttgaaagacttggtcgtaacaaagaaattggtgaacagatcgccaagaaaattcaacaggcaggggaatatgctaagcttgacccttaccgcgcggcaacccataacaaggggattatgaacggtatcgaagccgtcgttgccgcaacgggaaacgacacacgggctgtttccgcttctattcacgcatacgccgcccgtaatggcttgtaccaaggtttaacggattggcagatcaagggcgataaactggttggtaaattaacagtcccactggctgtggcgactgtcggtggcgcgtcgaacatattaccaaaagccaaagcttccctcgccatgctggatattgattccgcaaaagaactggcccaagtgatcgccgcggtaggtttagcacagaatctggcggcgttacgtgcattagtgacagaaggcattcagaaaggacacatgggcttgcaagcacgttctttagcgatttcgataggtgccatcggtgaggagatagagcaagtcgcgaaaaaactgcgtgaagctgaaaaaatgaatcagcaaacggcaatacagattttagaaaaaattcgcgagaaatga

SEQ ID NO: 6 - DNA, 1155 bp, Enterococcus faecalis mvaE
atgaaaatcggtattgaccgtctgtccttcttcatcccgaatttgtatttggacatgactgagctggcagaatcacgcggggatgatccagctaaatatcatattggaatcggacaagatcagatggcagtgaatcgcgcaaacgaggacatcataacactgggtgcaaacgctgcgagtaagatcgtgacagagaaagaccgcgagttgattgatatggtaatcgttggcacggaatcaggaattgaccactccaaagcaagcgccgtgattattcaccatctccttaaaattcagtcgttcgcccgttctttcgaggtaaaagaagcttgctatggcggaactgctgccctgcacatggcgaaggagtatgtcaaaaatcatccggagcgtaaggtcttggtaattgcgtcagacatcgcgcgttatggtttggccagcggaggagaagttactcaaggcgtgggggccgtagccatgatgattacacaaaacccccggattctttcgattgaagacgatagtgtttttctcacagaggatatctatgatttctggcggcctgattactccgagttccctgtagtggacgggcccctttcaaactcaacgtatatagagagttttcagaaagtttggaaccggcacaaggaattgtccggaagagggctggaagattatcaagctattgcttttcacataccctatacgaagatgggtaagaaagcgctccagagtgttttagaccaaaccgatgaagataaccaggagcgcttaatggctagatatgaggagtctattcgctatagccggagaattggtaacctgtacacaggcagcttgtaccttggtcttacaagcttgttggaaaactctaaaagtttacaaccgggagatcggatcggcctcttttcctatggcagtggtgcggtgtccgagttctttaccgggtatttagaagaaaattaccaagagtacctgttcgctcaaagccatcaagaaatgctggatagccggactcggattacggtcgatgaatacgagaccatcttttcagagactctgccagaacatggtgaatgcgccgaatatacgagcgacgtccccttttctataaccaagattgagaacgacattcgttattataaaatctga

SEQ ID NO: 7 - DNA, 2448 bp, Listeria grayi DSM 20601 mvaS
atggaagaagtggtaattatagatgcacgtcggactccgattggtaaatatcacgggtcgttgaagaagttttcagcggtggcgctggggacggccgtggctaaagacatgttcgaacgcaaccagaaaatcaaagaggagatcgcgcaggtcataattggtaatgtcttgcaggcaggaaatggccagaaccccgcgcggcaagttgctcttcaatcagggttgtccgttgacattcccgcttctacaattaacgaggtttgtgggtctggtttgaaagctatcttgatgggcatggaacaaatccaactcggcaaagcgcaagtagtgctggcaggcggcattgaatcaatgacaaatgcgccaagcctgtcccactataacaaggcggaggatacgtatagtgtcccagtgtcgagcatgacactggatggtctgacagacgcattttctagtaaacctatgggattaacagcggaaaacgtcgcacagcgctacggtatctcccgtgaggcgcaagatcaattcgcatatcaatctcagatgaaagcagcaaaagcgcaggcagaaaacaaattcgctaaggaaattgtgccactggcgggtgaaactaaaaccatcacagctgacgaagggatcagatcccaaacaacgatggagaaactggcaagtctcaaacctgtttttaaaaccgatggcactgtaaccgcagggaatgctagcaccattaatgacggggccgcccttgtgctgcttgctagcaaaacttactgcgaaactaatgacataccgtaccttgcgacaatcaaagaaattgttgaagttggaatcgatccggagattatgggcatctctccgataaaagcgatacaaacattgttacaaaatcaaaaagttagcctcgaagatattggagtttttgaaataaatgaagcctttgccgcaagtagcatagtggttgaatctgagttgggattagatccggctaaagttaaccgttatgggggtggtatatccttaggtcatgcaattggggcaaccggcgctcgcctggccacttcactggtgtatcaaatgcaggagatacaagcacgttatggtattgcgagcctgtgcgttggtggtggacttggactggcaatgcttttagaacgtccaactattgagaaggctaaaccgacagacaaaaagttctatgaattgtcaccagctgaacggttgcaagagctggaaaatcaacagaaaatcagttctgaaactaaacagcagttatctcagatgatgcttgccgaggacactgcaaaccatttgatagaaaatcaaatatcagagattgaactcccaatgggcgtcgggatgaacctgaaggttgatgggaaagcctatgttgtgccaatggcgacggaagagccgtccgtcatcgcggccatgtctaatggtgccaaaatggccggcgaaattcacactcagtcgaaagaacggctgctcagaggtcagattgttttcagcgcgaagaatccgaatgaaatcgaacagagaatagctgagaaccaagctttgattttcgaacgtgccgaacagtcctatccttccattgtgaaaagagagggaggtctccgccgcattgcacttcgtcattttcctgccgattctcagcaggagtctgcggaccagtccacatttttatcagtggacctttttgtagatgtgaaagacgcgatgggggcaaatatcataaatgcaatacttgagggcgtcgcagccctgtttcgcgaatggttccccaatgaggaaattcttttttctattctctcgaacttggctacggagagcttagtcacggctgtttgtgaagtcccatttagtgcacttagcaagagaggtggtgcaacggtggcccagaaaattgtgcaggcgtcgctcttcgcaaagacagacccataccgcgcagtgacccacaacaaagggattatgaacggtgtagaggctgttatgcttgccacaggcaacgacacgcgcgcagtctcagccgcttgtcatggatacgcagcgcgcaccggtagctatcagggtctgactaactggacgattgagtcggatcgcctggtaggcgagataacactgccgctggccatcgctacagttggaggcgctaccaaagtgttgcccaaagctcaagcggcactggagattagtgatgttcactcttctcaagagcttgcagccttagcggcgtcagtaggtttagtacaaaatctcgcggccctgcgcgcactggtttccgaaggtatacaaaaagggcacatgtccatgcaagcccggtctctcgcaatcgcggtcggtgctgaaaaagccgagatcgagcaggtcgccgaaaagttgcggcagaacccgccaatgaatcagcagcaggcgctccgttttcttggcgagatccgcgaacaatga

SEQ ID NO: 8 - DNA, 1155 bp, Enterococcus faecium mvaS
atgaacgtcggcattgacaaaattaattttttcgttccaccgtattatctggatatggtcgacctggcccacgcacgcgaagtggacccgaacaaatttacaattggaattggacaggatcagatggctgtgagcaaaaagacgcacgatatcgtaacattcgcggctagtgccgcgaaggaaattttagaacctgaggacttgcaagctatagacatggttatagttggtaccgaatcgggcattgacgagagcaaagcatccgcggtcgttttacatcgtttgttgggcgtacaacctttcgctcgcagttttgaaattaaagaagcctgttacggggcaaccgcaggcattcagtttgccaagactcatatacaagcgaacccggagagcaaggtcctggtaattgcaagcgatatagctcggtatggtcttcggtcaggtggagagcccacacaaggcgcaggggcagttgctatgcttctcacggcaaatcccagaatcctgaccttcgaaaacgacaatctgatgttaacgcaggatatttatgacttctggagaccacttggtcacgcttaccctatggtagatggccacctttccaatcaagtctatattgacagttttaagaaggtctggcaagcacattgcgaacgcaatcaagcttctatatccgactatgccgcgattagttttcatattccgtatacaaaaatgggtaagaaagccctgctcgctgtttttgcagatgaagtggaaactgaacaggaacgcgttatggcacggtatgaagagtctatcgtatattcacgccggatcggcaacttgtatacgggatcattgtacctggggctgatatccttattggaaaacagttctcacctgtcggcgggcgaccggataggattgtttagttatgggagtggcgctgtcagcgaatttttctccggtcgtttagtggcaggctatgaaaatcaattgaacaaagaggcgcatacccagctcctggatcagcgtcagaagctttccatcgaagagtatgaggcgatttttacagattccttagaaattgatcaggatgcagcgttctcggatgacctgccatattccatccgcgagataaaaaacacgattcggtactataaggagagctga

SEQ ID NO: 9 - DNA, 2475 bp, Enterococcus gallinarum EG2 mvaS
atggaagaagttgtcatcattgacgcactgcgtactccaataggaaagtaccacggttcgctgaaagattacacagctgttgaactggggacagtagcagcaaaggcgttgctggcacgaaatcagcaagcaaaagaacacatagcgcaagttattattggcaacgtcctgcaagccggaagtgggcagaatccaggccgacaagtcagtttacagtcaggattgtcttctgatatccccgctagcacgatcaatgaagtgtgtggctcgggtatgaaagcgattctgatgggtatggagcaaattcagctgaacaaagcctctgtggtcttaacaggcggaattgaaagcatgaccaacgcgccgctgtttagttattacaacaaggctgaggatcaatattcggcgccggttagcacaatgatgcacgatggtctaacagatgctttcagttccaaaccaatgggcttaaccgcagagaccgtcgctgagagatatggaattacgcgtaaggaacaagatgaatttgcttatcactctcaaatgaaggcggccaaagcccaggcggcgaaaaagtttgatcaggaaattgtacccctgacggaaaaatccggaacggttctccaggacgaaggcatcagagccgcgacaacagtcgagaagctagctgagcttaaaacggtgttcaaaaaagacggaacagttacagcgggtaacgcctctacgataaatgatggcgctgctatggtattaatagcatcaaaatcttattgcgaagaacaccagattccttatctggccgttataaaggagatcgttgaggtgggttttgcccccgaaataatgggtatttcccccattaaggctatagacaccctgctgaaaaatcaagcactgaccatagaggatataggaatatttgagattaatgaagcctttgctgcgagttcgattgtggtagaacgcgagttgggcctggaccccaaaaaagttaatcgctatggcggtggtatatcactcggccacgcaattggggcgacgggagctcgcattgcgacgaccgttgcttatcagctgaaagatacccaggagcgctacggtatagcttccttatgcgttggtgggggtcttggattggcgatgcttctggaaaacccatcggccactgcctcacaaactaattttgatgaggaatctgcttccgaaaaaactgagaagaagaagttttatgcgctagctcctaacgaacgcttagcgtttttggaagcccaaggcgctattaccgctgctgaaaccctggtcttccaggagatgaccttaaacaaagagacagccaatcacttaatcgaaaaccaaatcagcgaagttgaaattcctttaggcgtgggcctgaacttacaggtgaatgggaaagcgtataatgttcctctggccacggaggaaccgtccgttatcgctgcgatgtcgaatggcgccaaaatggctggtcctattacaacaacaagtcaggagaggctgttacggggtcagattgtcttcatggacgtacaggacccagaagcaatattagcgaaagttgaatccgagcaagctaccattttcgcggtggcaaatgaaacatacccgtctatcgtgaaaagaggaggaggtctgcgtagagtcattggcaggaatttcagtccggccgaaagtgacttagccacggcgtatgtatcaattgacctgatggtagatgttaaggatgcaatgggtgctaatatcatcaatagtatcctagaaggtgttgcggaattgtttagaaaatggttcccagaagaagaaatcctgttctcaattctctccaatctcgcgacagaaagtctggtaacggcgacgtgctcagttccgtttgataaattgtccaaaactgggaatggtcgacaagtagctggtaaaatagtgcacgcggcggactttgctaagatagatccatacagagctgccacacacaataaaggtattatgaatggcgttgaagcgttaatcttagccaccggtaatgacacccgtgcggtgtcggctgcatgccacggttacgcggcacgcaatgggcgaatgcaagggcttacctcttggacgattatcgaagatcggctgataggctctatcacattacctttggctattgcgacagtggggggtgccacaaaaatcttgccaaaagcacaggccgccctggcgctaactggcgttgagacggcgtcggaactggccagcctggcggcgagtgtgggattagttcaaaatttggccgctttacgagcactagtgagcgagggcattcagcaagggcacatgagtatgcaagctagatccctggccattagcgtaggtgcgaaaggtactgaaatagagcaactagctgcgaagctgagggcagcgacgcaaatgaatcaggagcaggctcgtaaatttctgaccgaaataagaaattaa

SEQ ID NO: 10 - DNA, 1161 bp, Enterococcus casseliflavus mvaS
atgaacgttggaattgataaaatcaattttttcgttccgccctatttcattgatatggtggatctcgctcatgcaagagaagttgaccccaacaagttcactataggaataggccaagatcagatggcagtaaacaagaaaacgcaagatatcgtaacgttcgcgatgcacgccgcgaaggatattctgactaaggaagatttacaggccatagatatggtaatagtggggactgagtctgggatcgacgagagcaaggcaagtgctgtcgtattgcatcggcttttaggtattcagccttttgcgcgctcctttgaaattaaggaggcatgctatggggccactgccggccttcagtttgcaaaagctcatgtgcaggctaatccccagagcaaggtcctggtggtagcttccgatatagcacgctacggactggcatccggaggagaaccgactcaaggtgtaggtgctgtggcaatgttgatttccgctgatccagctatcttgcagttagaaaatgataatctcatgttgacccaagatatatacgatttttggcgcccggtcgggcatcaatatcctatggtagacggccatctgtctaatgccgtctatatagacagctttaaacaagtctggcaagcacattgcgagaaaaaccaacggactgctaaagattatgctgcattgtcgttccatattccgtacacgaaaatgggtaagaaagctctgttagcggtttttgcggaggaagatgagacagaacaaaagcggttaatggcacgttatgaagaatcaattgtatacagtcgtcggactggaaatctgtatactggctcactctatctgggcctgatttccttactggagaatagtagcagtttacaggcgaacgatcgcataggtctgtttagctatggttcaggggccgttgcggaatttttcagtggcctcttggtaccgggttacgagaaacaattagcgcaagctgcccatcaagctcttctggacgaccggcaaaaactgactatcgcagagtacgaagccatgtttaatgaaaccattgatattgatcaggaccagtcatttgaggatgacttactgtactccatcagagagatcaaaaacactattcgctactataacgaggagaatgaataa

SEQ ID NO: 11 - DNA, 51 bp, Artificial Sequence
  Synthetic Construct
atagcgaattcagaaggagatataccatggaagcacgtcgctctgcgaact

SEQ ID NO: 12 - DNA, 37 bp, Artificial Sequence
  Synthetic Construct
atacgcggatccttagcgttcaaacggcagaatcggt

(19) United States
(12) Patent Application Publication
(10) Pub. No.: US 2014/0128645 A1
(43) Pub. Date: May 8, 2014

(54) ENHANCED PRODUCTION OF ISOPRENE USING MARINE BACTERIAL CELLS

(71) Applicants: Danisco US Inc., (US); The Goodyear Tire & Rubber Company, Akron, OH (US)

(72) Inventors: Richard LA DUCA, Pleasanton, CA (US); Karl J. SANFORD, Cupertino, CA (US); Gregory M. WHITED, Belmont, CA (US)

(73) Assignees: The Goodyear Tire & Rubber Company, Akron, OH (US); DANISCO US INC., Palo Alto, CA (US)

(21) Appl. No.: 13/722,991

(22) Filed: Dec. 20, 2012

Related U.S. Application Data
(60) Provisional application No. 61/580,168, filed on Dec. 23, 2011.

(51) Int. Cl. C12P 5/00

(52) U.S. Cl. CPC C12P 5/007
USPC 585/16; 435/252.3; 435/167

(57) ABSTRACT

The invention provides for methods for the production of isoprene in recombinant marine bacterial cells via the heterologous expression of isoprene synthase enzymes.

### CROSS-REFERENCE TO RELATED APPLICATIONS

This application claims priority benefit of U.S. Provisional patent application No. 61/580,168, filed on Dec. 23, 2011. The content of that application is hereby incorporated by reference herein in its entirety.

### INCORPORATION BY REFERENCE

The Sequence Listing submitted in an ASCII text file, in accordance with 37 C.F.R. §1.821(c) and (e), is incorporated by herein by reference. The text file name is “643842004300.txt”, the date of creation of the text file is Dec. 20, 2012, and the size of the ASCII text file in bytes is 24,576.

### FIELD OF THE INVENTION

The present invention relates to cultured recombinant marine bacterial cells capable of producing isoprene and compositions that include these cultured cells, as well as methods for producing and using the same.

### BACKGROUND OF THE INVENTION

Isoprene (2-methyl-1,3-butadiene) is the critical starting material for a variety of synthetic polymers, most notably synthetic rubbers. Isoprene can be obtained by fractionating petroleum; however, the purification of this material is expensive and time-consuming. Petroleum cracking of the C5 stream of hydrocarbons produces only about 15% isoprene. About 800,000 tons per year of cis-polyisoprene are produced from the polymerization of isoprene; most of this polyisoprene is used in the tire and rubber industry. Isoprene is also copolymerized for use as a synthetic elastomer in other products such as footwear, mechanical products, medical products, sporting goods, and latex. Isoprene can also be naturally produced by a variety of microbial, plant, and animal species. In particular, two pathways have been identified for the natural biosynthesis of isoprene: the mevalonate (MVA) pathway and the non-mevalonate (DXP) pathway. The products of the mevalonate and non-mevalonate pathway are isopentenyl pyrophosphate (IPP) and dimethylallyl diphosphate (DMAPP). A key enzyme in the pathway is isoprene synthase (IspS), which converts the precursor DMAPP to isoprene.

Recent developments in the production of isoprene disclose methods for the production of isoprene at rates, titers, and purities that can be sufficient to meet the demands of robust commercial processes (see, for example, International Patent Application Publication No. WO 2009/076676 A2); however, alternate pathways to improve production and yields of isoprene in an efficient manner are still needed.

Renewable resources (e.g., biomass) traditionally have posed problems for degradation. The ability to use renewable resources is hindered due to the complexity of material (e.g., plants containing cellulose) found in such renewable resources and the challenge it poses for the degradation of the material into simple sugars (e.g., fructose, glucose, xylose, mannose, arabinose, or lactose) that can be subsequently used as energy sources by organisms (e.g., bacteria) for the production of an end product of interest (e.g., isoprene or ethanol). Consolidated bioprocessing in which the organism digesting the biomass also produces the end product of interest offers potential to be among the least expensive and most efficient routes to the bioprocessing of biomass for the generation of isoprene.

Engineered bacteria of interest that produce isoprene may involve their utilization of biomass (e.g., plants) as a carbon source for the purpose of producing a consolidated and efficient bioprocessing method in which the microorganism digesting the biomass also produces the end product of interest. However, the cell walls of plants are composed of a heterogenous mixture of complex polysaccharides that interact through covalent and noncovalent means. Complex polysaccharides of higher plant cell walls include, for example, cellulose (β-1,4-glucan) which generally makes up 35-50% of carbon found in cell wall components and can be found as cellulose microfibrils. These microfibrils are embedded in a matrix formed of hemicelluloses (including, e.g., xylans, arabinans, and mannans), pectins (e.g., galacturonans and galactans), and various other β-1,3 and β-1,4 glucans. These matrix polymers are often substituted with, for example, arabinose, galactose and/or xylose residues to yield highly complex arabinoxylans, arabinogalactans, galactomannans, and xyloglucans. The hemicellulose matrix is, in turn, surrounded by polyphenolic lignin.

The complexity of the matrix found in plants makes it difficult for a microrganism to directly degrade and utilize it. A consortium of different microorganisms is usually required to degrade cell wall polymers for the release of constituent monosaccharides. Furthermore, for industrial saccharification of cell walls, large amounts of primarily fungal cellulases are added to processed feedstock that has been treated with dilute sulfuric acid at high temperature and pressure to permeabilize the lignin and partially saccharify the hemicellulose constituents. Therefore, there is a commercial need for bacteria that are engineered to directly utilize biomass for the production of isoprene.

Provided herein are cultured recombinant marine bacterial cells, compositions of these cells, and methods of using these cells to increase production of isoprene directly from biomass. The invention provided herein addresses these problems and provides additional benefits as well.

Throughout this specification, various patents, patent applications and other types of publications (e.g., journal articles) are referenced. The disclosure of all patents, patent applications, and publications cited herein are hereby incorporated by reference in their entirety for all purposes.

### BRIEF SUMMARY OF THE INVENTION

The invention provided herein discloses, inter alia, compositions of matter comprising recombinant marine bacterial cells, recombinants marine bacterial cells, and methods of making and using these recombinant marine bacterial cells for the production of isoprene. In some aspects the recombinant microorganisms comprise an heterologous ispS gene for the increased production of isoprene.

Accordingly, in some aspects, provided herein is a recombinant marine bacterial cell capable of increased production of isoprene, the cell comprising one or more copies of a heterologous nucleic acid encoding an isoprene synthase, wherein said cell produces isoprene at a higher level than isoprene produced by a cell that does not comprise one or more copies of a heterologous nucleic acid encoding an isoprene synthase. In other aspects, the recombinant marine bacterial cell is a gram-positive bacterium or a gram-negative bacterium. In yet another aspect, the recombinant marine bacterial cell is a cellulolytic bacterium. In another aspect, the recombinant marine bacterial cell is an agarolytic bacterium. In a further aspect, the recombinant marine bacterial cell is an alginolytic bacterium. In some aspects, the recombinant marine bacterial cell is a glucanolytic bacterium. In other aspects, the recombinant marine bacterial cell is a chitinolytic bacterium. In another aspect, the recombinant marine bacterial cell is a pectinolytic bacterium. In yet another aspect, the recombinant marine bacterial cell is a xylanolytic bacterium. In other aspects, the recombinant marine bacterial cell is a mannanolytic bacterium. In some aspects, the recombinant marine bacterial cell is a marine γ-proteobacterium. In another aspect, the recombinant marine bacterial cell is a marine saprophytic bacterium. In some aspects, the recombinant marine bacterial cell is a Microbulbifer, a Marinobacterium or a Saccharophagus. In still another aspect, the recombinant marine bacterial cell is selected from the group consisting of Saccharophagus degradans 2-40, Microbulbifer hydrolyticus IRE-31 and Marinobacterium georgiense KW-40. In another aspect, the recombinant marine bacterial cell is Saccharophagus degradans 2-40 having the identifying characteristics of ATCC 43961. In some aspects, the recombinant marine bacterial cell is Microbulbifer hydrolyticus IRE-31 having the identifying characteristics of ATCC 700072. In other aspects, the recombinant marine bacterial cell is Marinobacterium georgiense KW-40 having the identifying characteristics of ATCC 700074. In some aspects, the recombinant marine bacterial cell is cultured in a medium comprising a carbon source selected from the group consisting of biomass, carbohydrates, sugar alcohols, and byproducts of biodiesel production. In another aspect, the recombinant marine bacterial cell is cultured in a medium containing biomass, wherein the biomass is selected from the group consisting of wood, crops, waste, and plants. In still yet another aspect, the recombinant marine bacterial cell is cultured in a medium containing carbohydrates, wherein the carbohydrates is selected from the group consisting of monosaccharides, disaccharides, oligosaccharides, and polysaccharides. In a further aspect, the recombinant marine bacterial cell is cultured in a medium containing carbohydrates, wherein the carbohydrates is selected from the group consisting of agar, agarose, alginate, chitin, cellulose, fucoidan, laminarin, pectin, pullulan, starch α-glucan, β-glucan, glucomannan, galactomannan, and xylan. In another aspect, the recombinant marine bacterial cell is cultured in a medium comprising a carbon source selected from the group consisting of glucose, glycerol, glycerine, dihydroxyacetone, yeast extract, biomass, molasses, sucrose, corn cob, algae, cellulose, xylan, pectin, agar, alginate, chitin, α-glucans, β-glucans, laminarin, glucomannan, galactomannan, march grass, and oil. In some aspects, the recombinant marine bacterial cell comprises one or more copies of a heterologous nucleotide sequence encoding a plant isoprene synthase. In other aspects, the recombinant marine bacterial cell comprises one or more copies of a heterologous nucleotide sequence encoding a poplar isoprene synthase polypeptide. In another aspect, the recombinant marine bacterial cell comprises one or more copies of a heterologous nucleotide sequence encoding a kudzu isoprene synthase polypeptide. In another aspect, the recombinant marine bacterial cell comprises one or more copies of a heterologous nucleotide sequence encoding a willow isoprene synthase polypeptide. In yet another aspect, the recombinant marine bacterial cell comprises one or more copies of a heterologous nucleotide sequence encoding a eucalyptus isoprene synthase polypeptide. In some aspects, the recombinant marine bacterial cell comprises one or more copies of a heterologous nucleotide sequence encoding an isoprene synthase from Pueraria or Populus or a hybrid, Populus alba×Populus tremula. In other aspects, the recombinant marine bacterial cell comprises one or more copies of a heterologous nucleotide sequence encoding an isoprene synthase from the group consisting of Pueraria montana or Pueraria lobata, Populus tremuloides, Populus alba, Populus nigra, and Populus trichocarpa. In still another aspect, the recombinant marine bacterial cell comprises one or more copies of a heterologous nucleotide sequence encoding the P. alba isoprene synthase of SEQ ID NO: 1. In one aspect, the recombinant marine bacterial cell comprises one or more copies of a heterologous nucleotide sequence encoding an isoprene synthase variant. In a further aspect, the recombinant marine bacterial cell further comprises a heterologous nucleic acid encoding for one or more MVA pathway polypeptide and/or one or more DXP pathway polypeptide. In still another aspect, the recombinant marine bacterial cell further comprises a heterologous nucleic acid encoding for one or more MVA pathway polypeptides and/or an endogenous polynucleotide sequence encoding for one or more DXP pathway polypeptides. In still a further aspect, the recombinant marine bacterial cell further comprises a heterologous nucleic acid encoding for one or more IDI polypeptides. In one aspect, the recombinant marine bacterial cell comprises any one or more copies of a heterologous nucleic acid and wherein the one or more copies of the polynucleotide sequence is overexpressed. In still another aspect, the heterologous nucleic acid is cloned into a multicopy plasmid. In some aspects, the heterologous nucleic acid is cloned into an IncQ or IncQ-like plasmid. In one aspect, the heterologous nucleic acid is placed under an inducible promoter or a constitutive promoter. In another aspect, any one or more of the heterologous nucleic acids is integrated into the chromosome of the bacterial cell.

Additionally, in some aspects, provided herein is a method of producing isoprene, comprising: culturing a recombinant marine bacterial cell comprising one or more copies of a heterologous nucleic acid encoding an isoprene synthase in under suitable culture conditions for production of isoprene, wherein said cell produces isoprene at a higher level than isoprene produced by a cell that does not comprise one or more copies of a heterologous sequence encoding an isoprene synthase; and producing the isoprene. In some aspects, the method further comprises recovering the isoprene. In another aspect, the method further comprises polymerizing the isoprene. In still other aspects, the cell is a gram-positive bacterium or a gram-negative bacterium. In one aspect, the cell is a cellulolytic bacterium. In another aspect, the cell is an agarolytic bacterium. In a further aspect, the cell is an alginolytic bacterium. In some aspects, the cell is a glucanolytic bacterium. In other aspects, the cell is a chitinolytic bacterium. In another aspect, the cell is a pectinolytic bacterium. In yet another aspect, the cell is a xylanolytic bacterium. In other aspects, the cell is a mannanolytic bacterium. In some aspects, the cell is a marine γ-proteobacterium. In another aspect, the cell is a marine saprophytic bacterium. In some aspects, the cell is a Microbulbifer, a Marinobacterium or a Saccharophagus. In still another aspect, the cell is selected from the group consisting of Saccharophagus degradans 2-40, Microbulbifer hydrolyticus IRE-31 and Marinobacterium georgiense KW-40. In another aspect, the cell is Saccharophagus degradans 2-40 having the identifying characteristics of ATCC 43961. In some aspects, the cell is Microbulbifer hydrolyticus IRE-31 having the identifying characteristics of ATCC 700072. In other aspects, the cell is Marinobacterium georgiense KW-40 having the identifying characteristics of ATCC 700074. In some aspects, the recombinant marine bacterial cell is cultured in a medium comprising a carbon source selected from the group consisting of biomass, carbohydrates, sugar alcohols, and byproducts of biodiesel production. In another aspect, the cell is cultured in a medium containing biomass, wherein the biomass is selected from the group consisting of wood, crops, waste, and plants. In still yet another aspect, the cell is cultured in a medium containing carbohydrates, wherein the carbohydrates is selected from the group consisting of monosaccharides, disaccharides, oligosaccharides, and polysaccharides. In a further aspect, the cell is cultured in a medium containing carbohydrates, wherein the carbohydrates is selected from the group consisting of agar, agarose, alginate, chitin, cellulose, fucoidan, laminarin, pectin, pullulan, starch α-glucan, β-glucan, glucomannan, galactomannan, and xylan. In another aspect, the cell is cultured in a medium comprising a carbon source selected from the group consisting of glucose, glycerol, glycerine, dihydroxyacetone, yeast extract, biomass, molasses, sucrose, corn cob, algae, cellulose, xylan, pectin, agar, alginate, chitin, α-glucans, β-glucans, laminarin, glucomannan, galactomannan, march grass, and oil. In some aspects, the isoprene synthase is a plant isoprene synthase. In other aspects, the plant isoprene synthase polypeptide is a poplar isoprene synthase polypeptide. In another aspect, the plant isoprene synthase polypeptide is a kudzu isoprene synthase polypeptide. In another aspect, the plant isoprene synthase polypeptide is a willow isoprene synthase polypeptide. In another aspect, the plant isoprene synthase polypeptide is a eucalyptus isoprene synthase polypeptide. In some aspects, the isoprene synthase is an isoprene synthase from Pueraria or Populus or a hybrid, Populus alba×Populus tremula. In other aspects, the isoprene synthase polypeptide is selected from the group consisting of Pueraria montana or Pueraria lobata, Populus tremuloides, Populus alba, Populus nigra, and Populus trichocarpa. In still another aspect, the isoprene synthase is the P. alba isoprene synthase of SEQ ID NO: 1. In some aspects, the isoprene synthase is an isoprene synthase variant. In another aspect, the cell further comprises a heterologous nucleic acid encoding for one or more MVA pathway polypeptide and/or one or more DXP pathway polypeptide. In yet another aspect, the cell further comprises a heterologous nucleic acid encoding for one or more MVA pathway polypeptide and/or an endogenous polynucleotide sequence encoding for one or more DXP pathway polypeptide. In a further aspect, any one or more copies of a heterologous nucleic acid is overexpressed. In a further aspect, the heterologous nucleic acid is cloned into a multicopy plasmid. In another aspect, the heterologous nucleic acid is cloned into an IncQ or IncQ-like plasmid. In some aspects, the heterologous nucleic acid is placed under an inducible promoter or a constitutive promoter. In other apsects, the heterologous nucleic acid is integrated into the chromosome of the bacterial cell.

Provided herein, in some aspects, is a composition comprising isoprene produced by the recombinant marine bacterial cell disclosed herein.

### BRIEF DESCRIPTION OF THE DRAWINGS

FIG. 1 is a western blot demonstrating PCR amplification of the ispS gene.

FIG. 2 is an SDS-PAGE gel demonstrating the expression of a protein of a molecular weight of about 60 kDa (arrow) in crude lysates from IPTG induced Rosetta cultures transformed with pET24a-ispS (Lane 2) or pMMB503EH-ispS (Lane 4) as compared to non-induced Rosetta cultures transformed with pET24a-ispS (Lane 1) or pMMB503EH-ispS (Lane 3).

FIG. 3 is an SDS-PAGE gel demonstrating the expression of a protein of a molecular weight of about 60 kDa (arrow) in crude lysates from either IPTG induced (Lane 2) or non-induced (Lane 1) Rosetta cells transformed with pET24a-ispS as compared to crude lysates from either IPTG induced (Lane 4) or non-induced (Lane 3) Saccharophagus degradans cultures transformed with pMMB503EH-ispS.

FIG. 4 is an SDS-PAGE gel demonstrating the expression of a protein of a molecular weight of about 60 kDa (arrow) in crude lysates from either IPTG induced (Lane 2) or non-induced (Lane 1) Rosetta cells transformed with pET24a-ispS as compared to crude lysates from either IPTG induced (Lane 4) or non-induced (Lane 3) Saccharophagus degradans cultures transformed with pMMB503EH-ispS grown on conventional media or from IPTG induced (Lane 6) or non-induced (Lane 5) Saccharophagus degradans cultures transformed with pMMB503EH-ispS grown on media with corn cob as the carbon source.

FIG. 5 is a western blot and Coomasie stained SDS-PAGE gel demonstrating the expression of isoprene synthase in harvested cell cultures. Western blot (left panel) depicts expression of isoprene synthase protein in supernatents from either IPTG induced (Lane 3) or non-induced (Lane 5) Saccharophagus degradans cultures transformed with pMMB503EH-ispS as compared to harvested pellets from IPTG induced (Lane 4) or non-induced (Lane 6) Saccharophagus degradans cultures transformed with pMMB503EH-ispS. Lane 1 contains molecular weight marker and Lane 2 contains 0.2 μg of purified isoprene synthase as positive control. Coomasie gel (right panel) depicts total protein levels in supernatents from either IPTG induced (Lane 9) or non-induced (Lane 11) Saccharophagus degradans cultures transformed with pMMB503EH-ispS as compared to harvested pellets from IPTG induced (Lane 10) or non-induced (Lane 12) Saccharophagus degradans cultures transformed with pMMB503EH-ispS. Lane 7 contains molecular weight marker and Lane 8 contains 0.4 μg of purified isoprene synthase as positive control.

FIG. 6 is a western blot demonstrating the expression of isoprene synthase in harvested cell cultures. Western blot depicts expression of isoprene synthase protein in supernatents from either IPTG induced (Lane 6) or non-induced (Lane 10) Saccharophagus degradans cultures transformed with pMMB503EH-ispS as compared to whole cell lysate from IPTG induced (Lane 5) or non-induced (Lane 9) Saccharophagus degradans cultures transformed with pMMB503EH-ispS. Lane 1 contains molecular weight marker, Lane 2 contains 0.4 μg of purified isoprene synthase as positive control. Negative control is whole cell lysate (Lane 7) and supernatant (Lane 8) from Saccharophagus degradans cultures transformed with empty pMMB503EH vector.

### DETAILED DESCRIPTION

The invention provided herein discloses, inter alia, compositions and methods for the production of isoprene in recombinant marine bacterial cells that have been engineered to express an isoprene synthase. As further detailed herein, in one aspect, the present invention is directed to a recombinant marine bacterial cell capable of increased production of isoprene, the cell comprising one or more copies of a heterologous nucleic acid encoding an isoprene synthase, wherein said cell produces isoprene at a higher level as compared to cells that do not comprise one or more copies of a heterologous nucleic acid encoding an isoprene synthase. The invention is based in part on the discovery that heterologous expression of the P. alba ispS gene in the biomass-degrading marine bacteria results in isoprene production directly from biomass in comparison to cells that do not express the P. alba ispS gene. In some aspects, the marine bacterium is Saccharophagus degradans.

In certain aspects, the invention provides recombinant cells capable of enhanced production of isoprene, wherein the cells comprise one or more heterologous nucleic acids encoding a polypeptide having isoprene synthase activity and one or more nucleic acids encoding one or more polypeptides of the MVA pathway and/or DXP pathway, wherein the cells produce increased amounts of isoprene compared to cells that do not comprise the one or more heterologous nucleic acids encoding a polypeptide having isoprene synthase activity.

The headings provided herein are not limitations of the various aspects or embodiments of the invention, which can be had by reference to the specification as a whole. Accordingly, the terms defined immediately below are more fully defined by reference to the specification as a whole. Nonetheless, in order to facilitate understanding of the invention, a number of terms are defined below.

### General Techniques

The practice of the present invention will employ, unless otherwise indicated, conventional techniques of molecular biology (including recombinant techniques), microbiology, cell biology, biochemistry, and immunology, which are within the skill of the art. Such techniques are explained fully in the literature, “Molecular Cloning: A Laboratory Manual”, third edition (Sambrook et al., 2001); “Oligonucleotide Synthesis” (M. J. Gait, ed., 1984); “Animal Cell Culture: A practical approach”, third edition (J. R. Masters, ed., 2000); “Methods in Enzymology” (Academic Press, Inc.); “Current Protocols in Molecular Biology” (F. M. Ausubel et al., eds., 1987, and periodic updates); “PCR: The Polymerase Chain Reaction”, (Mullis et al., eds., 1994). Singleton et al., Dictionary of Microbiology and Molecular Biology 3rd revised ed., J. Wiley & Sons (New York, N.Y. 2006), and March's Advanced Organic Chemistry Reactions, Mechanisms and Structure 6th ed., John Wiley & Sons (New York, N.Y. 2007), provide one skilled in the art with a general guide to many of the terms used in the present application.

### DEFINITIONS

As used herein, the terms “isoprene synthase,” “isoprene synthase variant”, and “IspS,” refer to enzymes that catalyze the elimination of pyrophosphate from diemethylallyl diphosphate (DMAPP) to form isoprene. An “isoprene synthase” may be a wild type sequence or an isoprene synthase variant.

The term “isoprene” refers to 2-methyl-1,3-butadiene (CAS#78-79-5). It can be the direct and final volatile C5 hydrocarbon product from the elimination of pyrophosphate from DMAPP. It may not involve the linking or polymerization of IPP molecules to DMAPP molecules. The term “isoprene” is not generally intended to be limited to its method of production unless indicated otherwise herein.

As used herein, a “nucleic acid” refers to two or more deoxyribonucleotides and/or ribonucleotides covalently joined together in either single or double-stranded form.

By “recombinant nucleic acid” is meant a nucleic acid of interest that is free of one or more nucleic acids (e.g., genes) which, in the genome occurring in nature of the organism from which the nucleic acid of interest is derived, flank the nucleic acid of interest. The term therefore includes, for example, a recombinant DNA which is incorporated into a vector, into an autonomously replicating plasmid or virus, or into the genomic DNA of a prokaryote or eukaryote, or which exists as a separate molecule (e.g., a cDNA, a genomic DNA fragment, or a cDNA fragment produced by PCR or restriction endonuclease digestion) independent of other sequences.

By “heterologous nucleic acid” is meant a nucleic acid sequence derived from a different organism, species or strain than the host cell. In some embodiments, the heterologous nucleic acid is not identical to a wild-type nucleic acid that is found in the same host cell in nature.

An “endogenous nucleic acid” is a nucleic acid whose nucleic acid sequence is naturally found in the host cell. In some embodiments, an endogenous nucleic acid is identical to a wild-type nucleic acid that is found in the host cell in nature. In some embodiments, one or more copies of endogenous nucleic acids are introduced into a host cell.

As used herein, an “expression control sequence” means a nucleic acid sequence that directs transcription of a nucleic acid of interest. An expression control sequence can be a promoter, such as a constitutive or an inducible promoter, or an enhancer. An expression control sequence can be “native” or heterologous. A native expression control sequence is derived from the same organism, species, or strain as the gene being expressed. A heterologous expression control sequence is derived from a different organism, species, or strain as the gene being expressed. An “inducible promoter” is a promoter that is active under environmental or developmental regulation.

By “operably linked” is meant a functional linkage between a nucleic acid expression control sequence (such as a promoter) and a second nucleic acid sequence, wherein the expression control sequence directs transcription of the nucleic acid corresponding to the second sequence.

As used herein, the term “polypeptides” includes polypeptides, proteins, peptides, fragments of polypeptides, and fusion polypeptides.

As used herein, an “isolated polypeptide” is not part of a library of polypeptides, such as a library of 2, 5, 10, 20, 50 or more different polypeptides and is separated from at least one component with which it occurs in nature. An isolated polypeptide can be obtained, for example, by expression of a recombinant nucleic acid encoding the polypeptide.

By “heterologous polypeptide” is meant a polypeptide encoded by a nucleic acid sequence derived from a different organism, species, or strain than the host cell. In some embodiments, a heterologous polypeptide is not identical to a wild-type polypeptide that is found in the same host cell in nature.

As used herein, the terms “minimal medium” or “minimal media” refer to growth medium containing the minimum nutrients possible for cell growth, generally without the presence of amino acids. Minimal medium typically contains: (1) a carbon source for bacterial growth; (2) various salts, which can vary among bacterial species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

As used herein, the term “mass yield” refers to the mass of the product produced by the bacterial cells divided by the mass of the glucose consumed by the bacterial cells multiplied by 100.

By “specific productivity,” it is meant the mass of the product produced by the bacterial cell divided by the product of the time for production, the cell density, and the volume of the culture.

As used herein, the term “headspace” refers to the vapor/air mixture trapped above a solid or liquid sample in a sealed vessel.

By “titer,” it is meant the mass of the product produced by the bacterial cells divided by the volume of the culture.

As used herein, the term “cell productivity index (CPI)” refers to the mass of the product produced by the bacterial cells divided by the mass of the bacterial cells produced in the culture.

As used herein, the singular terms “a,” “an,” and “the” include the plural reference unless the context clearly indicates otherwise.

Unless defined otherwise herein, all technical and scientific terms used herein have the same meaning as commonly understood by one of ordinary skill in the art to which this invention pertains.

It is intended that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Reference to “about” a value or parameter herein also includes (and describes) embodiments that are directed to that value or parameter per se.

It is understood that all aspects and embodiments of the invention described herein include “comprising,” “consisting,” and “consisting essentially of” aspects and embodiments. It is to be understood that methods or compositions “consisting essentially of” the recited elements include only the specified steps or materials and those that do not materially affect the basic and novel characteristics of those methods and compositions.

It is to be understood that this invention is not limited to the particular methodology, protocols, and reagents described, as these may vary, depending upon the context they are used by those of skill in the art.

### Recombinant Marine Bacterial Cells Expressing an Isoprene Synthase

Isoprene (2-methyl-1,3-butadiene) is an important organic compound used in a wide array of applications. For instance, isoprene is employed as an intermediate or a starting material in the synthesis of numerous chemical compositions and polymers, including in the production of synthetic rubber. Isoprene is also an important biological material that is synthesized naturally by many animals, plants, and bacteria. Building a strain (prokaryotic or eukaryotic) capable of producing commercially viable levels of isoprene requires optimization of part of or the entire DXP or MVA pathway or both MVA and DXP pathways. A key enzyme in the pathway is isoprene synthase (IspS), which converts the precursor DMAPP to isoprene.

Engineered bacteria of interest that produce isoprene may involve their utilization of biomass (e.g., plants) as a carbon source for the purpose of producing a consolidated and efficient bioprocessing method in which the microorganism digesting the biomass also produces the end product of interest. However, the cell walls of plants are composed of a heterogenous mixture of complex polysaccharides that interact through covalent and noncovalent means. Complex polysaccharides of higher plant cell walls include, for example, cellulose (β-1,4-glucan) which generally makes up 35-50% of carbon found in cell wall components and can be found as cellulose microfibrils. These microfibrils are embedded in a matrix formed of hemicelluloses (including, e.g., xylans, arabinans, and mannans), pectins (e.g., galacturonans and galactans), and various other β-1,3 and β-1,4 glucans. These matrix polymers are often substituted with, for example, arabinose, galactose and/or xylose residues to yield highly complex arabinoxylans, arabinogalactans, galactomannans, and xyloglucans. The hemicellulose matrix is, in turn, surrounded by polyphenolic lignin.

The complexity of the matrix found in plants makes it difficult for a microrganism to directly degrade and utilize it. A consortium of different microorganisms is usually required to degrade cell wall polymers for the release of constituent monosaccharides. Furthermore, for industrial saccharification of cell walls, large amounts of primarily fungal cellulases are added to processed feedstock that has been treated with dilute sulfuric acid at high temperature and pressure to permeabilize the lignin and partially saccharify the hemicellulose constituents. Therefore, there is a commercial need for bacteria that are engineered to directly utilize biomass for the production of isoprene.

Thus, in certain embodiments, the recombinant bacterial cell of the present invention is a recombinant marine bacterial cell capable of increased production of isoprene, wherein the cell comprises one or more copies a heterologous nucleic acid encoding an isoprene synthase, wherein said cell produces isoprene at a higher level than isoprene produced by a cell that does not comprise one or more copies of a heterologous nucleic acid encoding the isoprene synthase. In one embodiment, the recombinant marine bacterial cell is a marine saphrophytic bacterium. In another embodiment, the recombinant bacterial cell is Saccharophagus degradans 2-40. In some embodiments, the recombinant marine bacterial cell is cultured in a medium comprising biomass to produce isoprene. In other embodiments, the isoprene synthase is a plant isoprene synthase. In a further embodiment, the isoprene synthase is the P. alba synthase. In another embodiment, the isoprene synthase is an isoprene synthase variant. In certain embodiments, the recombinant bacterial cell further comprises a heterologous nucleic acid encoding one or more MVA pathway polypeptide and/or one or more DXP pathway polypeptide.

### Isoprene Synthase Nucleic Acids and Polypeptides

In some aspects of the invention, the recombinant marine bacterial cells described in any of the compositions or methods described herein further comprise one or more nucleic acids encoding an isoprene synthase polypeptide or a polypeptide having isoprene synthase activity. In some aspects, the isoprene synthase polypeptide is an endogenous polypeptide. In some aspects, the endogenous nucleic acid encoding an isoprene synthase polypeptide is operably linked to a constitutive promoter. In some aspects, the endogenous nucleic acid encoding an isoprene synthase polypeptide is operably linked to an inducible promoter. In some aspects, the endogenous nucleic acid encoding an isoprene synthase polypeptide is operably linked to a strong promoter. In some aspects, more than one endogenous nucleic acid encoding an isoprene synthase polypeptide is used (e.g, 2, 3, 4, or more copies of an endogenous nucleic acid encoding an isoprene synthase polypeptide). In a particular aspect, the cells are engineered to overexpress the endogenous isoprene synthase pathway polypeptide relative to wild-type cells. In some aspects, the endogenous nucleic acid encoding an isoprene synthase polypeptide is operably linked to a weak promoter.

In some aspects, the isoprene synthase polypeptide is a heterologous polypeptide. In some aspects, the cells comprise more than one copy of a heterologous nucleic acid encoding an isoprene synthase polypeptide. In some aspects, the heterologous nucleic acid encoding an isoprene synthase polypeptide is operably linked to a constitutive promoter. In some aspects, the heterologous nucleic acid encoding an isoprene synthase polypeptide is operably linked to an inducible promoter. In some aspects, the heterologous nucleic acid encoding an isoprene synthase polypeptide is operably linked to a strong promoter. In some aspects, the heterologous nucleic acid encoding an isoprene synthase polypeptide is operably linked to a weak promoter. In some aspects, the isoprene synthase polypeptide is a polypeptide from Pueraria or Populus or a hybrid such as Populus alba×Populus tremula. In some aspects, the isoprene synthase polypeptide is from Eucalyptus.

The nucleic acids encoding an isoprene synthase polypeptide(s) can be integrated into a genome of the host cells or can be stably expressed in the cells. The nucleic acids encoding an isoprene synthase polypeptide(s) can additionally be on a vector.

Exemplary isoprene synthase nucleic acids include nucleic acids that encode a polypeptide, fragment of a polypeptide, peptide, or fusion polypeptide that has at least one activity of an isoprene synthase polypeptide. Isoprene synthase polypeptides convert dimethylallyl diphosphate (DMAPP) into isoprene. Exemplary isoprene synthase polypeptides include polypeptides, fragments of polypeptides, peptides, and fusions polypeptides that have at least one activity of an isoprene synthase polypeptide. Exemplary isoprene synthase polypeptides and nucleic acids include naturally-occurring polypeptides and nucleic acids from any of the source organisms described herein. In addition, variants of isoprene synthase can possess improved activity such as improved enzymatic activity. In some aspects, an isoprene synthase variant has other improved properties, such as improved stability (e.g., thermo-stability), and/or improved solubility.

Standard methods can be used to determine whether a polypeptide has isoprene synthase polypeptide activity by measuring the ability of the polypeptide to convert DMAPP into isoprene in vitro, in a cell extract, or in vivo. Isoprene synthase polypeptide activity in the cell extract can be measured, for example, as described in Silver et al., J. Biol. Chem. 270:13010-13016, 1995. In one exemplary assay, DMAPP (Sigma) can be evaporated to dryness under a stream of nitrogen and rehydrated to a concentration of 100 mM in 100 mM potassium phosphate buffer pH 8.2 and stored at −20 0 C. To perform the assay, a solution of 5 μL of 1M MgCl₂, 1 mM (250 μg/ml) DMAPP, 65 μL of Plant Extract Buffer (PEB) (50 mM Tris-HCl, pH 8.0, 20 mM MgCl₂, 5% glycerol, and 2 mM DTT) can be added to 25 μL of cell extract in a 20 ml Headspace vial with a metal screw cap and teflon coated silicon septum (Agilent Technologies) and cultured at 370 C for 15 minutes with shaking. The reaction can be quenched by adding 200 μL of 250 mM EDTA and quantified by GC/MS.

In some aspects, the isoprene synthase polypeptide is a plant isoprene synthase polypeptide or a variant thereof. In some aspects, the isoprene synthase polypeptide is an isoprene synthase from Pueraria or a variant thereof. In some aspects, the isoprene synthase polypeptide is an isoprene synthase from Populus or a variant thereof. In some aspects, the isoprene synthase polypeptide is a poplar isoprene synthase polypeptide or a variant thereof. In some aspects, the isoprene synthase polypeptide is a kudzu isoprene synthase polypeptide or a variant thereof. In some aspects, the isoprene synthase polypeptide is a willow isoprene synthase polypeptide or a variant thereof. In some aspects, the isoprene synthase polypeptide is a eucalyptus isoprene synthase polypeptide or a variant thereof. In some aspects, the isoprene synthase polypeptide is a polypeptide from Pueraria or Populus or a hybrid, Populus alba×Populus tremula, or a variant thereof. In some aspects, the isoprene synthase polypeptide is from Robinia, Salix, or Melaleuca or variants thereof.

In some embodiments, the parent isoprene synthase is from the family Fabaceae, the family Salicaceae, or the family Fagaceae. In some aspects, the isoprene synthase polypeptide or nucleic acid is a polypeptide or nucleic acid from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), English Oak (Quercus robur) (Zimmer et al., WO 98/02550), or a variant thereof. In some aspects, the isoprene synthase polypeptide is an isoprene synthase from Pueraria montana, Pueraria lobata, Populus tremuloides, Populus alba, Populus nigra, or Populus trichocarpa or a variant thereof. In some aspects, the isoprene synthase polypeptide is an isoprene synthase from Populus alba or a variant thereof. In some aspects, the isoprene synthase is Populus balsamifera (Genbank JN173037), Populus deltoides (Genbank JN173039), Populus fremontii (Genbank JN173040), Populus granididenta (Genbank JN173038), Salix (Genbank JN173043), Robinia pseudoacacia (Genbank JN173041), Wisteria (Genbank JN173042), Eucalyptus globulus (Genbank AB266390) or Melaleuca alterniflora (Genbank AY279379) or variant thereof. In some aspects, the nucleic acid encoding the isoprene synthase (e.g., isoprene synthase from Populus alba or a variant thereof) is codon optimized.

In some aspects, the isoprene synthase nucleic acid or polypeptide is a naturally-occurring polypeptide or nucleic acid (e.g., naturally-occurring polypeptide or nucleic acid from Populus). In some aspects, the isoprene synthase nucleic acid or polypeptide is not a wild-type or naturally-occurring polypeptide or nucleic acid. In some aspects, the isoprene synthase nucleic acid or polypeptide is a variant of a wild-type or naturally-occurring polypeptide or nucleic acid (e.g., a variant of a wild-type or naturally-occurring polypeptide or nucleic acid from Populus).

In some aspects, the isoprene synthase polypeptide is a variant. In some aspects, the isoprene synthase polypeptide is a variant of a wild-type or naturally occurring isoprene synthase. In some aspects, the variant has improved activity such as improved catalytic activity compared to the wild-type or naturally occurring isoprene synthase. The increase in activity (e.g., catalytic activity) can be at least about any of 10%, 20%, 30%, 40%, 50%, 60%, 70%, 80%, 90%, or 95%. In some aspects, the increase in activity such as catalytic activity is at least about any of 1 fold, 2 folds, 5 folds, 10 folds, 20 folds, 30 folds, 40 folds, 50 folds, 75 folds, or 100 folds. In some aspects, the increase in activity such as catalytic activity is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the variant has improved solubility compared to the wild-type or naturally occurring isoprene synthase. The increase in solubility can be at least about any of 10%, 20%, 30%, 40%, 50%, 60%, 70%, 80%, 90%, or 95%. The increase in solubility can be at least about any of 1 fold, 2 folds, 5 folds, 10 folds, 20 folds, 30 folds, 40 folds, 50 folds, 75 folds, or 100 folds. In some aspects, the increase in solubility is about 10% to about 100 folds (e.g., about 20% to about 100 folds, about 50% to about 50 folds, about 1 fold to about 25 folds, about 2 folds to about 20 folds, or about 5 folds to about 20 folds). In some aspects, the isoprene synthase polypeptide is a variant of naturally occurring isoprene synthase and has improved stability (such as thermo-stability) compared to the naturally occurring isoprene synthase.

In some aspects, the variant has at least about 10%, at least about 20%, at least about 30%, at least about 40%, at least about 50%, at least about 60%, at least about 70%, at least about 80%, at least about 90%, at least about 100%, at least about 110%, at least about 120%, at least about 130%, at least about 140%, at least about 150%, at least about 160%, at least about 170%, at least about 180%, at least about 190%, at least about 200% of the activity of a wild-type or naturally occurring isoprene synthase. The variant can share sequence similarity with a wild-type or naturally occurring isoprene synthase. In some aspects, a variant of a wild-type or naturally occurring isoprene synthase can have at least about any of 40%, 50%, 60%, 70%, 75%, 80%, 90%, 91%, 92%, 93%, 94%, 95%, 96%, 97%, 98%, 99%, 99.5%, or 99.9% amino acid sequence identity as that of the wild-type or naturally occurring isoprene synthase. In some aspects, a variant of a wild-type or naturally occurring isoprene synthase has any of about 70% to about 99.9%, about 75% to about 99%, about 80% to about 98%, about 85% to about 97%, or about 90% to about 95% amino acid sequence identity as that of the wild-type or naturally occurring isoprene synthase.

In some aspects, the variant comprises a mutation in the wild-type or naturally occurring isoprene synthase. In some aspects, the variant has at least one amino acid substitution, at least one amino acid insertion, and/or at least one amino acid deletion. In some aspects, the variant has at least one amino acid substitution. In some aspects, the number of differing amino acid residues between the variant and wild-type or naturally occurring isoprene synthase can be one or more, e.g. 1, 2, 3, 4, 5, 10, 15, 20, 30, 40, 50, or more amino acid residues. Naturally occurring isoprene synthases can include any isoprene synthases from plants, for example, kudzu isoprene synthases, poplar isoprene synthases, English oak isoprene synthases, willow isoprene synthases, and eucalyptus isoprene synthases. In some aspects, the variant is a variant of isoprene synthase from Populus alba. In some aspects, the variant of isoprene synthase from Populus alba has at least one amino acid substitution, at least one amino acid insertion, and/or at least one amino acid deletion. In some aspects, the variant is a truncated Populus alba isoprene synthase. In some aspects, the nucleic acid encoding variant (e.g., variant of isoprene synthase from Populus alba) is codon optimized (for example, codon optimized based on host cells where the heterologous isoprene synthase is expressed).

In certain embodiments described herein, an isoprene synthase gene encoding an isoprene synthase polypeptide derived from P. alba can be used. An example of such an isoprene synthase polypeptide is the polypeptide encoded by a gene having the polynucleotide sequence of SEQ ID NO: 1. In various embodiments, the isoprene synthase has at least about 50%, at least about 60%, at least about 70%, at least about 80%, at least about 90%, at least about 92%, at least about 94%, at least about 96%, at least about 98%, at least about 99% sequence identity with MEA P. alba. In other embodiments, isoprene synthase has at least about 50%, at least about 60%, at least about 70%, at least about 80%, at least about 90%, at least about 92%, at least about 94%, at least about 96%, at least about 98%, at least about 99% sequence identity with full-length P. alba or complete P. alba.

Sequence of P. alba ispS
(SEQ ID NO: 1)
ATGGAAGCACGTCGCTCTGCGAACTACGAACCTAACAGCTGGGACTATGATTA
CCTGCTGTCCTCCGACACGGACGAGTCCATCGAAGTATACAAAGACAAAGCGAAAAAG
CTGGAAGCCGAAGTTCGTCGCGAGATTAATAACGAAAAAGCAGAATTTCTGACCCTGC
TGGAACTGATTGACAACGTCCAGCGCCTGGGCCTGGGTTACCGTTTCGAGTCTGATATC
CGTGGTGCGCTGGATCGCTTCGTTTCCTCCGGCGGCTTCGATGCGGTAACCAAGACTTC
CCTGCACGGTACGGCACTGTCTTTCCGTCTGCTGCGTCAACACGGTTTTGAGGTTTCTCA
GGAAGCGTTCAGCGGCTTCAAAGACCAAAACGGCAACTTCCTGGAGAACCTGAAGGAA
GATATCAAAGCTATCCTGAGCCTGTACGAGGCCAGCTTCCTGGCTCTGGAAGGCGAAA
ACATCCTGGACGAGGCGAAGGTTTTCGCAATCTCTCATCTGAAAGAACTGTCTGAAGA
AAAGATCGGTAAAGAGCTGGCAGAACAGGTGAACCATGCACTGGAACTGCCACTGCAT
CGCCGTACTCAGCGTCTGGAAGCAGTATGGTCTATCGAGGCCTACCGTAAAAAGGAGG
ACGCGAATCAGGTTCTGCTGGAGCTGGCAATTCTGGATTACAACATGATCCAGTCTGTA
TACCAGCGTGATCTGCGTGAAACGTCCCGTTGGTGGCGTCGTGTGGGTCTGGCGACCAA
ACTGCACTTTGCTCGTGACCGCCTGATTGAGAGCTTCTACTGGGCCGTGGGTGTAGCAT
TCGAACCGCAATACTCCGACTGCCGTAACTCCGTCGCAAAAATGTTTTCTTTCGTAACC
ATTATCGACGATATCTACGATGTATACGGCACCCTGGACGAACTGGAGCTGTTTACTGA
TGCAGTTGAGCGTTGGGACGTAAACGCCATCAACGACCTGCCGGATTACATGAAACTG
TGCTTTCTGGCTCTGTATAACACTATTAACGAAATCGCCTACGACAACCTGAAAGATAA
AGGTGAGAACATCCTGCCGTATCTGACCAAAGCCTGGGCTGACCTGTGCAACGCTTTCC
TGCAAGAAGCCAAGTGGCTGTACAACAAATCTACTCCGACCTTTGACGACTACTTCGGC
AACGCATGGAAATCCTCTTCTGGCCCGCTGCAACTGGTGTTCGCTTACTTCGCTGTCGT
GCAGAACATTAAAAAGGAAGAGATCGAAAACCTGCAAAAATACCATGACACCATCTCT
CGTCCTTCCCATATCTTCCGTCTGTGCAATGACCTGGCTAGCGCGTCTGCGGAAATTGC
GCGTGGTGAAACCGCAAATAGCGTTTCTTGTTACATGCGCACTAAAGGTATCTCCGAAG
AACTGGCTACCGAAAGCGTGATGAATCTGATCGATGAAACCTGGAAAAAGATGAACAA
GGAAAAACTGGGTGGTAGCCTGTTCGCGAAACCGTTCGTGGAAACCGCGATCAACCTG
GCACGTCAATCTCACTGCACTTATCATAACGGCGACGCGCATACCTCTCCGGATGAGCT
GACCCGCAAACGCGTTCTGTCTGTAATCACTGAACCGATTCTGCCGTTTGAACGCTAA

Suitable isoprene synthases include, but are not limited to, those identified by Genbank Accession Nos. AY341431, AY316691, AB198180, AJ294819.1, EU693027.1, EF638224.1, AM410988.1, EF147555.1, AY279379, AJ457070, and AY182241. Types of isoprene synthases which can be used in any one of the compositions or methods including methods of making microorganisms encoding isoprene synthase described herein are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/124146, WO2010/078457, and WO2010/148256, U.S. Patent Application Publication No.: 2010/0086978, U.S. patent application Ser. No. 13/283,564, and Sharkey et al., “Isoprene Synthase Genes Form A Monophyletic Clade Of Acyclic Terpene Synthases In The Tps-B Terpene Synthase Family”, Evolution (2012) (available on line at DOI: 10.1111/evo.12013), the contents of which are expressly incorporated herein by reference in their entirety with respect to the isoprene synthases and isoprene synthase variants.

Any one of the promoters described herein (e.g., promoters described herein and identified in the Examples of the present disclosure including inducible promoters and constitutive promoters) can be used to drive expression of any of the isoprene synthases described herein.

### MVA Pathway Nucleic Acids and Polypeptides

The complete MVA pathway can be subdivided into two groups: an upper and lower pathway. In the upper portion of the MVA pathway, acetyl Co-A produced during cellular metabolism is converted to mevalonate via the actions of polypeptides having either: (a) (i) thiolase activity or (ii) acetoacetyl-CoA synthase activity, (b) HMG-CoA reductase, and (c) HMG-CoA synthase enzymatic activity. First, acetyl Co-A is converted to acetoacetyl CoA via the action of a thiolase or an acetoacetyl-CoA synthase (which utilizes acetyl-CoA and malonyl-CoA). Next, acetoacetyl-CoA is converted to 3-hydroxy-3-methylglutaryl-CoA (HMG-CoA) by the enzymatic action of HMG-CoA synthase. This Co-A derivative is reduced to mevalonate by HMG-CoA reductase, which is the rate-limiting step of the mevalonate pathway of isoprenoid production. In the lower MVA pathway, mevalonate is then converted into mevalonate-5-phosphate via the action of mevalonate kinase which is subsequently transformed into 5-diphosphomevalonate by the enzymatic activity of phosphomevalonate kinase. Finally, IPP is formed from 5-diphosphomevalonate by the activity of the enzyme mevalonate-5-pyrophosphate decarboxylase.

Exemplary MVA pathway polypeptides that can be used in conjunction with an ispS gene, but are not limited to: 3-hydroxy-3-methylglutaryl-CoA synthase (HMG-CoA synthase) polypeptides (e.g., an enzyme encoded by mvaS), 3-hydroxy-3-methylglutaryl-CoA reductase (HMG-CoA reductase) polypeptides (e.g., enzyme encoded by mvaR or enzyme encoded by mvaE that has been modified to be thiolase-deficient but still retains its reductase activity), mevalonate kinase (MVK) polypeptides, phosphomevalonate kinase (PMK) polypeptides, diphosphomevalonte decarboxylase (MVD) polypeptides, phosphomevalonate decarboxylase (PMDC) polypeptides, isopentenyl phosphate kinase (IPK) polypeptides, IPP isomerase polypeptides, IDI polypeptides, and polypeptides (e.g., fusion polypeptides) having an activity of two or more MVA pathway polypeptides. In particular, MVA pathway polypeptides include polypeptides, fragments of polypeptides, peptides, and fusions polypeptides that have at least one activity of an MVA pathway polypeptide. Exemplary MVA pathway nucleic acids include nucleic acids that encode a polypeptide, fragment of a polypeptide, peptide, or fusion polypeptide that has at least one activity of an MVA pathway polypeptide. Exemplary MVA pathway polypeptides and nucleic acids include naturally-occurring polypeptides and nucleic acids from any of the source organisms described herein. In addition, variants of MVA pathway polypeptide that confer the result of better isoprene production can also be used as well.

Non-limiting examples of MVA pathway polypeptides which can be used are described in International Patent Application Publication No. WO2009/076676; WO2010/003007, WO2010/031062 and WO2010/148150, the contents of which are expressly incorporated herein by reference in their entirety with respect to MVA pathway polypeptides.

### Acetoacetyl-CoA Synthase Nucleic Acids and Polypeptides

The acetoacetyl-CoA synthase gene (aka nphT7) is a gene encoding an enzyme having the activity of synthesizing acetoacetyl-CoA from malonyl-CoA and acetyl-CoA and having minimal activity (e.g., no activity) of synthesizing acetoacetyl-CoA from two acetyl-CoA molecules. See, e.g., Okamura et al., PNAS Vol 107, No. 25, pp. 11265-11270 (2010), the contents of which are expressly incorporated herein for teaching about nphT7. An acetoacetyl-CoA synthase gene from an actinomycete of the genus Streptomyces CL190 strain was described in JP Patent Publication (Kokai) No. 2008-61506 A and US2010/0285549. Acetoacetyl-CoA synthase can also be referred to as acetyl CoA:malonyl CoA acyltransferase. A representative acetoacetyl-CoA synthase (or acetyl CoA:malonyl CoA acyltransferase) that can be used is Genbank AB540131.1.

In any of the aspects or embodiments described herein, an enzyme that has the ability to synthesize acetoacetyl-CoA from malonyl-CoA and acetyl-CoA can be used. Non-limiting examples of such an enzyme are described herein. In certain embodiments described herein, an acetoacetyl-CoA synthase gene derived from an actinomycete of the genus Streptomyces having the activity of synthesizing acetoacetyl-CoA from malonyl-CoA and acetyl-CoA can be used. An example of such an acetoacetyl-CoA synthase gene is the gene encoding a protein having the amino acid sequence of SEQ ID NO: 2. Such a protein having the amino acid sequence of SEQ ID NO: 2 corresponds to an acetoacetyl-CoA synthase having activity of synthesizing acetoacetyl-CoA from malonyl-CoA and acetyl-CoA and having no activity of synthesizing acetoacetyl-CoA from two acetyl-CoA molecules.

Sequence of acetoacetyl-CoA synthase
(SEQ ID NO: 2)
MTDVRFRIIGTGAYVPERIVSNDEVGAPAGVDDDWITRKTGIRQ
RRWAADDQATSDLATAAGRAALKAAGITPEQLTVIAVATSTPDRPQPPTA
AYVQHHLGATGTAAFDVNAVCSGTVFALSSVAGTLVYRGGYALVIGADLY
SRILNPADRKTVVLFGDGAGAMVLGPTSTGTGPIVRRVALHTFGGLTDLI
RVPAGGSRQPLDTDGLDAGLQYFAMDGREVRRFVTEHLPQLIKGFLHEAG
VDAADISHFVPHQANGVMLDEVFGELHLPRATMHRTVETYGNTGAASIPI
TMDAAVRAGSFRPGELVLLAGFGGGMAASFALIEW.

In one embodiment, acetoacetyl-CoA synthase of the present invention synthesizes acetoacetyl-CoA from malonyl-CoA and acetyl-CoA via an irreversible reaction. The use of acetoacetyl-CoA synthase to generate acetyl-CoA provides an additional advantage in that this reaction is irreversible while acetoacetyl-CoA thiolase enzyme's action of synthesizing acetoacetyl-CoA from two acetyl-CoA molecules is reversible. Consequently, the use of acetoacetyl-CoA synthase to synthesize acetoacetyl-CoA from malonyl-CoA and acetyl-CoA can result in significant improvement in productivity for isoprene compared with using thiolase to generate the end same product.

Furthermore, the use of acetoacetyl-CoA synthase to produce isoprene provides another advantage in that acetoacetyl-CoA synthase can convert malonyl CoA to acetyl CoA via decarboxylation of the malonyl CoA. Thus, stores of starting substrate are not limited by the starting amounts of acetyl CoA. The synthesis of acetoacetyl-CoA by acetoacetyl-CoA synthase can still occur when the starting substrate is only malonyl-CoA. In one embodiment, the pool of starting malonyl-CoA is increased by using host strains that have more malonyl-CoA. Such increased pools can be naturally occurring or be engineered by molecular manipulation. See, for example Fowler, et. al, Applied and Environmental Microbiology, Vol. 75, No. 18, pp. 5831-5839 (2009).

In one embodiment, the gene encoding a protein having the amino acid sequence of SEQ ID NO: 2 can be obtained by a nucleic acid amplification method (e.g., PCR) with the use of genomic DNA obtained from an actinomycete of the Streptomyces sp. CL190 strain as a template and a pair of primers that can be designed with reference to JP Patent Publication (Kokai) No. 2008-61506A.

As described herein, an acetoacetyl-CoA synthase gene for use in the present invention is not limited to a gene encoding a protein having the amino acid sequence of SEQ ID NO: 2 from an actinomycete of the Streptomyces sp. CL190 strain. Any gene encoding a protein having the ability to synthesize acetoacetyl-CoA from malonyl-CoA and acetyl-CoA and which does not synthesize acetoacetyl-CoA from two acetyl-CoA molecules can be used in the presently described methods. In certain embodiments, the acetoacetyl-CoA synthase gene can be a gene encoding a protein having an amino acid sequence with high similarity or substantially identical to the amino acid sequence of SEQ ID NO: 2 and having the function of synthesizing acetoacetyl-CoA from malonyl-CoA and acetyl-CoA. The expression “highly similar” or “substantially identical” refers to, for example, at least about 80% identity, at least about 85%, at least about 90%, at least about 91%, at least about 92%, at least about 93%, at least about 94%, at least about 95%, at least about 96%, at least about 97%, at least about 98%, and at least about 99% identity. As used above, the identity value corresponds to the percentage of identity between amino acid residues in a different amino acid sequence and the amino acid sequence of SEQ ID NO: 2, which is calculated by performing alignment of the amino acid sequence of SEQ ID NO: 2 and the different amino acid sequence with the use of a program for searching for a sequence similarity.

In other embodiments, the acetoacetyl-CoA synthase gene may be a gene encoding a protein having an amino acid sequence derived from the amino acid sequence of SEQ ID NO: 2 by substitution, deletion, addition, or insertion of 1 or more amino acid(s) and having the function of synthesizing acetoacetyl-CoA from malonyl-CoA and acetyl-CoA. Herein, the expression “more amino acids” refers to, for example, 2 to 30 amino acids, preferably 2 to 20 amino acids, more preferably 2 to 10 amino acids, and most preferably 2 to 5 amino acids.

In still other embodiments, the acetoacetyl-CoA synthase gene may consist of a polynucleotide capable of hybridizing to a portion or the entirety of a polynucleotide having a nucleotide sequence complementary to the nucleotide sequence encoding the amino acid sequence of SEQ ID NO: 2 under stringent conditions and capable of encoding a protein having the function of synthesizing acetoacetyl-CoA from malonyl-CoA and acetyl-CoA. Herein, hybridization under stringent conditions corresponds to maintenance of binding under conditions of washing at 60.degree. C. 2.times.SSC. Hybridization can be carried out by conventionally known methods such as the method described in J. Sambrook et al. Molecular Cloning, A Laboratory Manual, 3rd Ed., Cold Spring Harbor Laboratory (2001).

As described herein, a gene encoding an acetoacetyl-CoA synthase having an amino acid sequence that differs from the amino acid sequence of SEQ ID NO: 2 can be isolated from potentially any organism, for example, an actinomycete that is not obtained from the Streptomyces sp. CL190 strain. In addition, acetoacetyl-CoA synthase genes for use herein can be obtained by modifying a polynucleotide encoding the amino acid sequence of SEQ ID NO: 2 by a method known in the art. Mutagenesis of a nucleotide sequence can be carried out by a known method such as the Kunkel method or the gapped duplex method or by a method similar to either thereof. For instance, mutagenesis may be carried out with the use of a mutagenesis kit (e.g., product names; Mutant-K and Mutant-G (TAKARA Bio)) for site-specific mutagenesis, product name; an LA PCR in vitro Mutagenesis series kit (TAKARA Bio), and the like.

The activity of an acetoacetyl-CoA synthase having an amino acid sequence that differs from the amino acid sequence of SEQ ID NO: 2 can be evaluated as described below. Specifically, a gene encoding a protein to be evaluated is first introduced into a host cell such that the gene can be expressed therein, followed by purification of the protein by a technique such as chromatography. Malonyl-CoA and acetyl-CoA are added as substrates to a buffer containing the obtained protein to be evaluated, followed by, for example, incubation at a desired temperature (e.g., 10° C. to 60° C.). After the completion of reaction, the amount of substrate lost and/or the amount of product (acetoacetyl-CoA) produced are determined. Thus, it is possible to evaluate whether or not the protein being tested has the function of synthesizing acetoacetyl-CoA from malonyl-CoA and acetyl-CoA and to evaluate the degree of synthesis. In such case, it is possible to examine whether or not the protein has the activity of synthesizing acetoacetyl-CoA from two acetyl-CoA molecules by adding acetyl-CoA alone as a substrate to a buffer containing the obtained protein to be evaluated and determining the amount of substrate lost and/or the amount of product produced in a similar manner.

### Nucleic Acids Encoding Polypeptides of the Upper MVA Pathway

The upper portion of the MVA pathway uses acetyl Co-A produced during cellular metabolism as the initial substrate for conversion to mevalonate via the actions of polypeptides having either: (a) (i) thiolase activity or (ii) acetoacetyl-CoA activity, (b) HMG-CoA reductase, and (c) HMG-CoA synthase enzymatic activity. First, acetyl Co-A is converted to acetoacetyl CoA via the action of a thiolase or an acetoacetyl-CoA synthase (which utilizes acetyl-CoA and malonyl-CoA). Next, acetoacetyl-CoA is converted to 3-hydroxy-3-methylglutaryl-CoA (HMG-CoA) by the enzymatic action of HMG-CoA synthase. This Co-A derivative is reduced to mevalonate by HMG-CoA reductase, which is the rate-limiting step of the mevalonate pathway of isoprenoid production.

Non-limiting examples of upper MVA pathway polypeptides include acetyl-CoA acetyltransferase (AA-CoA thiolase) polypeptides, acetoacetyl-CoA synthase polypeptides, 3-hydroxy-3-methylglutaryl-CoA synthase (HMG-CoA synthase) polypeptides, 3-hydroxy-3-methylglutaryl-CoA reductase (HMG-CoA reductase) polypeptides. Upper MVA pathway polypeptides can include polypeptides, fragments of polypeptides, peptides, and fusions polypeptides that have at least one activity of an upper MVA pathway polypeptide. Exemplary upper MVA pathway nucleic acids include nucleic acids that encode a polypeptide, fragment of a polypeptide, peptide, or fusion polypeptide that has at least one activity of an upper MVA pathway polypeptide. Exemplary MVA pathway polypeptides and nucleic acids include naturally-occurring polypeptides and nucleic acids from any of the source organisms described herein. Thus, it is contemplated herein that any gene encoding an upper MVA pathway polypeptide can be used in the present invention.

In certain embodiments, various options of mvaE and mvaS genes from L. grayi, E. faecium, E. gallinarum, E. casseliflavus and/or E. faecalis alone or in combination with one or more other mvaE and mvaS genes encoding proteins from the upper MVA pathway are contemplated within the scope of the invention. In other embodiments, an acetoacetyl-CoA synthase gene is contemplated within the scope of the present invention in combination with one or more other genes encoding: (i) 3-hydroxy-3-methylglutaryl-CoA synthase (HMG-CoA synthase) polypeptides and 3-hydroxy-3-methylglutaryl-CoA reductase (HMG-CoA reductase) polypeptides. Thus, in certain aspects, any of the combinations of genes contemplated in can be expressed in recombinant cells in any of the ways described herein.

Additional non-limiting examples of upper MVA pathway polypeptides which can be used herein are described in International Patent Application Publication No. WO2009/076676; WO2010/003007 and WO2010/148150.

Genes Encoding mvaE and mvaS Polypeptides

In certain embodiments, various options of mvaE and mvaS genes from L. grayi, E. faecium, E. gallinarum, E. casseliflavus and/or E. faecalis alone or in combination with one or more other mvaE and mvaS genes encoding proteins from the upper MVA pathway are contemplated within the scope of the invention. In L. grayi, E. faecium, E. gallinarum, E. casseliflavus, and E. faecalis, the mvaE gene encodes a polypeptide that possesses both thiolase and HMG-CoA reductase activities. In fact, the mvaE gene product represented the first bifunctional enzyme of IPP biosynthesis found in eubacteria and the first example of HMG-CoA reductase fused to another protein in nature (Hedl, et al., J Bacteriol. 2002 April; 184(8): 2116-2122). The mvaS gene, on the other hand, encodes a polypeptide having an HMG-CoA synthase activity.

Accordingly, recombinant cells (e.g., E. coli) can be engineered to express one or more mvaE and mvaS genes from L. grayi, E. faecium, E. gallinarum, E. casseliflavus and/or E. faecalis, to produce mevalonate. The one or more mvaE and mvaS genes can be expressed on a multicopy plasmid. The plasmid can be a high copy plasmid, a low copy plasmid, or a medium copy plasmid. Alternatively, the one or more mvaE and mvaS genes can be integrated into the host cell's chromosome. For both heterologous expression of the one or more mvaE and mvaS genes on a plasmid or as an integrated part of the host cell's chromosome, expression of the genes can be driven by either an inducible promoter or a constitutively expressing promoter. The promoter can be a strong driver of expression, it can be a weak driver of expression, or it can be a medium driver of expression of the one or more mvaE and mvaS genes.

Exemplary mvaE Polypeptides and Nucleic Acids

The mvaE gene encodes a polypeptide that possesses both thiolase and HMG-CoA reductase activities. The thiolase activity of the polypeptide encoded by the mvaE gene converts acetyl Co-A to acetoacetyl CoA whereas the HMG-CoA reductase enzymatic activity of the polypeptide converts 3-hydroxy-3-methylglutaryl-CoA to mevalonate. Exemplary mvaE polypeptides and nucleic acids include naturally-occurring polypeptides and nucleic acids from any of the source organisms described herein as well as mutant polypeptides and nucleic acids derived from any of the source organisms described herein that have at least one activity of a mvaE polypeptide.

Mutant mvaE polypeptides include those in which one or more amino acid residues have undergone an amino acid substitution while retaining mvaE polypeptide activity (i.e., the ability to convert acetyl Co-A to acetoacetyl CoA as well as the ability to convert 3-hydroxy-3-methylglutaryl-CoA to mevalonate). The amino acid substitutions can be conservative or non-conservative and such substituted amino acid residues can or cannot be one encoded by the genetic code. The standard twenty amino acid “alphabet” has been divided into chemical families based on similarity of their side chains. Those families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). A “conservative amino acid substitution” is one in which the amino acid residue is replaced with an amino acid residue having a chemically similar side chain (i.e., replacing an amino acid having a basic side chain with another amino acid having a basic side chain). A “non-conservative amino acid substitution” is one in which the amino acid residue is replaced with an amino acid residue having a chemically different side chain (i.e., replacing an amino acid having a basic side chain with another amino acid having an aromatic side chain).

Amino acid substitutions in the mvaE polypeptide can be introduced to improve the functionality of the molecule. For example, amino acid substitutions that increase the binding affinity of the mvaE polypeptide for its substrate, or that improve its ability to convert acetyl Co-A to acetoacetyl CoA and/or the ability to convert 3-hydroxy-3-methylglutaryl-CoA to mevalonate can be introduced into the mvaE polypeptide. In some aspects, the mutant mvaE polypeptides contain one or more conservative amino acid substitutions.

In one aspect, mvaE proteins that are not degraded or less prone to degradation can be used for the production of isoprene. Examples of gene products of mvaEs that are not degraded or less prone to degradation which can be used include, but are not limited to, those from the organisms E. faecium, E. gallinarum, E. casseliflavus, E. faecalis, and L. grayi. One of skill in the art can express mvaE protein in E. coli BL21 (DE3) and look for absence of fragments by any standard molecular biology techniques. For example, absence of fragments can be identified on Safestain stained SDS-PAGE gels following His-tag mediated purification or when expressed in mevalonate, isoprene or isoprenoid producing E. coli BL21 using the methods of detection described herein.

Standard methods, such as those described in Hedl et al., (J Bacteriol. 2002, April; 184(8): 2116-2122) can be used to determine whether a polypeptide has mvaE activity, by measuring acetoacetyl-CoA thiolase as well as HMG-CoA reductase activity. In an exemplary assay, acetoacetyl-CoA thiolase activity is measured by spectrophotometer to monitor the change in absorbance at 302 nm that accompanies the formation or thiolysis of acetoacetyl-CoA. Standard assay conditions for each reaction to determine synthesis of acetoacetyl-CoA, are 1 mM acetyl-CoA, 10 mM MgCl₂, 50 mM Tris, pH 10.5 and the reaction is initiated by addition of enzyme. Assays can employ a final volume of 200 μl. For the assay, 1 enzyme unit (eu) represents the synthesis or thiolysis in 1 min of 1 μmol of acetoacetyl-CoA. In another exemplary assay, of HMG-CoA reductase activity can be monitored by spectrophotometer by the appearance or disappearance of NADP(H) at 340 nm. Standard assay conditions for each reaction measured to show reductive deacylation of HMG-CoA to mevalonate are 0.4 mM NADPH, 1.0 mM (R,S)-HMG-CoA, 100 mM KCl, and 100 mM KₓPO₄, pH 6.5. Assays employ a final volume of 200 μl. Reactions are initiated by adding the enzyme. For the assay, 1 eu represents the turnover, in 1 min, of 1 μmol of NADP(H). This corresponds to the turnover of 0.5 μmol of HMG-CoA or mevalonate.

Exemplary mvaE nucleic acids include nucleic acids that encode a polypeptide, fragment of a polypeptide, peptide, or fusion polypeptide that has at least one activity of a mvaE polypeptide. Exemplary mvaE polypeptides and nucleic acids include naturally-occurring polypeptides and nucleic acids from any of the source organisms described herein as well as mutant polypeptides and nucleic acids derived from any of the source organisms described herein. Exemplary mvaE nucleic acids include, for example, mvaE nucleic acids isolated from Listeria grayi_DSM 20601, Enterococcus faecium, Enterococcus gallinarum EG2, Enterococcus faecalis, and/or Enterococcus casseliflavus. The mvaE nucleic acid encoded by the Listeria grayi_DSM 20601 mvaE gene can have a 99%, 98%, 97%, 96%, 95%, 95%, 93%, 92%, 91%, 90%, 89%, 88%, 87%, 86%, or 85% sequence identity to SEQ ID NO: 3. The mvaE nucleic acid encoded by the Enterococcus faecium mvaE gene can have a 99%, 98%, 97%, 96%, 95%, 95%, 93%, 92%, 91%, 90%, 89%, 88%, 87%, 86%, or 85% sequence identity to SEQ ID NO: 4. The mvaE nucleic acid encoded by the Enterococcus gallinarum EG2 mvaE gene can have a 99%, 98%, 97%, 96%, 95%, 95%, 93%, 92%, 91%, 90%, 89%, 88%, 87%, 86%, or 85% sequence identity to SEQ ID NO:5. The mvaE nucleic acid encoded by the Enterococcus casseliflavus mvaE gene can have a 99%, 98%, 97%, 96%, 95%, 95%, 93%, 92%, 91%, 90%, 89%, 88%, 87%, 86%, or 85% sequence identity to SEQ ID NO:6. The mvaE nucleic acid encoded by the Enterococcus faecalis mvaE gene can have a 99%, 98%, 97%, 96%, 95%, 95%, 93%, 92%, 91%, 90%, 89%, 88%, 87%, 86%, or 85% sequence identity to the mvaE gene previously disclosed in E. coli to produce mevalonate (see US 2005/0287655 A1; Tabata, K. and Hashimoto, S.-I. Biotechnology Letters 26: 1487-1491, 2004).

Sequence of Listeria grayi DSM 20601 mvaE
SEQ ID NO: 3
atggttaaagacattgtaataattgatgccctccgtactcccatcggtaagtaccgcggtcagctctcaaagatgacggcggtggaattgggaacc
gcagttacaaaggctctgttcgagaagaacgaccaggtcaaagaccatgtagaacaagtcatttttggcaacgttttacaggcagggaacggcc
agaatcccgcccgtcagatcgcccttaattctggcctgtccgcagagataccggcttcgactattaaccaggtgtgtggttctggcctgaaagcaa
taagcatggcgcgccaacagatcctactcggagaagcggaagtaatagtagcaggaggtatcgaatccatgacgaatgcgccgagtattacat
attataataaagaagaagacaccctctcaaagcctgttcctacgatgaccttcgatggtctgaccgacgcgtttagcggaaagattatgggtttaac
agccgaaaatgttgccgaacagtacggcgtatcacgtgaggcccaggacgcctttgcgtatggatcgcagatgaaagcagcaaaggcccaag
aacagggcattttcgcagctgaaatactgcctcttgaaataggggacgaagttattactcaggacgagggggttcgtcaagagaccaccctcga
aaaattaagtctgcttcggaccatttttaaagaagatggtactgttacagcgggcaacgcctcaacgatcaatgatggcgcctcagccgtgatcatt
gcatcaaaggagtttgctgagacaaaccagattccctaccttgcgatcgtacatgatattacagagataggcattgatccatcaataatgggcattg
ctcccgtgagtgcgatcaataaactgatcgatcgtaaccaaattagcatggaagaaatcgatctctttgaaattaatgaggcatttgcagcatcctc
ggtggtagttcaaaaagagttaagcattcccgatgaaaagatcaatattggcggttccggtattgcactaggccatcctcttggcgccacaggag
cgcgcattgtaaccaccctagcgcaccagttgaaacgtacacacggacgctatggtattgcctccctgtgcattggcggtggccttggcctagca
atattaatagaagtgcctcaggaagatcagccggttaaaaaattttatcaattggcccgtgaggaccgtctggctagacttcaggagcaagccgtg
atcagcccagctacaaaacatgtactggcagaaatgacacttcctgaagatattgccgacaatctgatcgaaaatcaaatatctgaaatggaaatc
cctcttggtgtggctttgaatctgagggtcaatgataagagttataccatcccactagcaactgaggaaccgagtgtaatcgctgcctgtaataatg
gtgcaaaaatggcaaaccacctgggcggttttcagtcagaattaaaagatggtttcctgcgtgggcaaattgtacttatgaacgtcaaagaacccg
caactatcgagcatacgatcacggcagagaaagcggcaatttttcgtgccgcagcgcagtcacatccatcgattgtgaaacgaggtgggggtct
aaaagagatagtagtgcgtacgttcgatgatgatccgacgttcctgtctattgatctgatagttgatactaaagacgcaatgggcgctaacatcatta
acaccattctcgagggtgtagccggctttctgagggaaatccttaccgaagaaattctgttctctattttatctaattacgcaaccgaatcaattgtga
ccgccagctgtcgcataccttacgaagcactgagtaaaaaaggtgatggtaaacgaatcgctgaaaaagtggctgctgcatctaaatttgcccag
ttagatccttatcgagctgcaacccacaacaaaggtattatgaatggtattgaggccgtcgttttggcctcaggaaatgacacacgggcggtcgc
ggcagccgcacatgcgtatgcttcacgcgatcagcactatcggggcttaagccagtggcaggttgcagaaggcgcgttacacggggagatca
gtctaccacttgcactcggcagcgttggcggtgcaattgaggtcttgcctaaagcgaaggcggcattcgaaatcatggggatcacagaggcga
aggagctggcagaagtcacagctgcggtagggctggcgcaaaacctggcggcgttaagagcgcttgttagtgaaggaatacagcaaggtcac
atgtcgctccaggctcgctctcttgcattatcggtaggtgctacaggcaaggaagttgaaatcctggccgaaaaattacagggctctcgtatgaat
caggcgaacgctcagaccatactcgcagagatcagatcgcaaaaagttgaattgtga
Sequence of Enterococcus faecium mvaE
SEQ ID NO: 4
atgaccatgaacgttggaatcgataaaatgtcattctttgttccaccttactttgtggacatgactgatctggcagtagcacgggatgtcgatcccaat
aagtttctgattggtattggccaggaccagatggcagttaatccgaaaacgcaggatattgtgacatttgccacaaatgctgccaaaaacatactgt
cagctgaggaccttgataaaattgatatggtcatagtcggcaccgagagtggaatcgatgaatccaaagcgagtgccgtagtgcttcacaggttg
ctcggtatccagaagtttgctcgctcctttgaaatcaaagaagcctgttatgggggtaccgcggctttacagttcgctgtaaaccacattaggaatc
atcctgaatcaaaggttcttgtagttgcatcagatatcgcgaaatacggcctggcttctggaggtgaaccaacgcaaggtgcaggcgctgtggct
atgctcgtctcaactgaccctaagatcattgctttcaacgacgatagcctcgcgcttacacaagatatctatgacttctggcgaccagttggacatga
ctatcctatggtcgacgggcctcttagtacagagacctacatccagtcatttcagaccgtatggcaggaatacacaaaacggtcgcagcatgcac
tggcagactttgctgcccttagctttcatatcccgtatactaaaatgggcaaaaaggcgctgcttgcaatccttgaaggcgaatcagaggaggctc
agaaccgtatactagcaaaatatgaaaagagtatagcctactccagaaaggcgggtaacctgtataccggtagcctgtatctaggacttatttcact
tctggaaaatgcagaagaccttaaagctggtgatttaataggcctcttttcttacggttccggtgctgttgcggagtttttctcaggaaggctggttga
ggactatcaggaacagctacttaaaacaaaacatgccgaacagctggcccatagaaagcaactgacaatcgaggagtacgaaacgatgttctc
cgatcgcttggacgtggacaaagacgccgaatacgaagacacattagcttatagcatttcgtcagtccgaaacaccgtacgtgagtacaggagtt
ga
Sequence of Enterococcus gallinarum EG2 mvaE
SEQ ID NO: 5
atgaaagaagtggttatgattgatgcggctcgcacacccattgggaaatacagaggtagtcttagtccttttacagcggtggagctggggacact
ggtcacgaaagggctgctggataaaacaaagcttaagaaagacaagatagaccaagtgatattcggcaatgtgcttcaggcaggaaacggaca
aaacgttgcaagacaaatagccctgaacagtggcttaccagttgacgtgccggcgatgactattaacgaagtttgcgggtccggaatgaaagcg
gtgattttagcccgccagttaatacagttaggggaggcagagttggtcattgcagggggtacggagtcaatgtcacaagcacccatgctgaaac
cttaccagtcagagaccaacgaatacggagagccgatatcatcaatggttaatgacgggctgacggatgcgttttccaatgctcacatgggtctta
ctgccgaaaaggtggcgacccagttttcagtgtcgcgcgaggaacaagaccggtacgcattgtccagccaattgaaagcagcgcacgcggttg
aagccggggtgttctcagaagagattattccggttaagattagcgacgaggatgtcttgagtgaagacgaggcagtaagaggcaacagcacttt
ggaaaaactgggcaccttgcggacggtgttttctgaagagggcacggttaccgctggcaatgcttcaccgctgaatgacggcgctagtgtcgtg
attcttgcatcaaaagaatacgcggaaaacaataatctgccttacctggcgacgataaaggaggttgcggaagttggtatcgatccttctatcatgg
gtattgccccaataaaggccattcaaaagttaacagatcggtcgggcatgaacctgtccacgattgatctgttcgaaattaatgaagcattcgcgg
catctagcattgttgtttctcaagagctgcaattggacgaagaaaaagtgaatatctatggcggggcgatagctttaggccatccaatcggcgcaa
gcggagcccggatactgacaaccttagcatacggcctcctgcgtgagcaaaagcgttatggtattgcgtcattatgtatcggcggtggtcttggtc
tggccgtgctgttagaagctaatatggagcagacccacaaagacgttcagaagaaaaagttttaccagcttaccccctccgagcggagatcgca
gcttatcgagaagaacgttctgactcaagaaacggcacttattttccaggagcagacgttgtccgaagaactgtccgatcacatgattgagaatca
ggtctccgaagtggaaattccaatgggaattgcacaaaattttcagattaatggcaagaaaaaatggattcctatggcgactgaagaaccttcagt
aatagcggcagcatcgaacggcgccaaaatctgcgggaacatttgcgcggaaacgcctcagcggcttatgcgcgggcagattgtcctgtctgg
caaatcagaatatcaagccgtgataaatgccgtgaatcatcgcaaagaagaactgattctttgcgcaaacgagtcgtacccgagtattgttaaacg
cgggggaggtgttcaggatatttctacgcgggagtttatgggttcttttcacgcgtatttatcaatcgactttctggtggacgtcaaggacgcaatgg
gggcaaacatgatcaactctattctcgaaagcgttgcaaataaactgcgtgaatggttcccggaagaggaaatactgttctccatcctgtcaaactt
cgctacggagtccctggcatctgcatgttgcgagattccttttgaaagacttggtcgtaacaaagaaattggtgaacagatcgccaagaaaattca
acaggcaggggaatatgctaagcttgacccttaccgcgcggcaacccataacaaggggattatgaacggtatcgaagccgtcgttgccgcaac
gggaaacgacacacgggctgtttccgcttctattcacgcatacgccgcccgtaatggcttgtaccaaggtttaacggattggcagatcaagggcg
ataaactggttggtaaattaacagtcccactggctgtggcgactgtcggtggcgcgtcgaacatattaccaaaagccaaagcttccctcgccatgc
tggatattgattccgcaaaagaactggcccaagtgatcgccgcggtaggtttagcacagaatctggcggcgttacgtgcattagtgacagaagg
cattcagaaaggacacatgggcttgcaagcacgttctttagcgatttcgataggtgccatcggtgaggagatagagcaagtcgcgaaaaaactg
cgtgaagctgaaaaaatgaatcagcaaacggcaatacagattttagaaaaaattcgcgagaaatga
Sequence of Enterococcus faecalis mvaE
SEQ ID NO: 6
atgaaaatcggtattgaccgtctgtccttcttcatcccgaatttgtatttggacatgactgagctggcagaatcacgcggggatgatccagctaaata
tcatattggaatcggacaagatcagatggcagtgaatcgcgcaaacgaggacatcataacactgggtgcaaacgctgcgagtaagatcgtgac
agagaaagaccgcgagttgattgatatggtaatcgttggcacggaatcaggaattgaccactccaaagcaagcgccgtgattattcaccatctcct
taaaattcagtcgttcgcccgttctttcgaggtaaaagaagcttgctatggcggaactgctgccctgcacatggcgaaggagtatgtcaaaaatcat
ccggagcgtaaggtcttggtaattgcgtcagacatcgcgcgttatggtttggccagcggaggagaagttactcaaggcgtgggggccgtagcc
atgatgattacacaaaacccccggattctttcgattgaagacgatagtgtttttctcacagaggatatctatgatttctggcggcctgattactccgagt
tccctgtagtggacgggcccctttcaaactcaacgtatatagagagttttcagaaagtttggaaccggcacaaggaattgtccggaagagggctg
gaagattatcaagctattgcttttcacataccctatacgaagatgggtaagaaagcgctccagagtgttttagaccaaaccgatgaagataaccag
gagcgcttaatggctagatatgaggagtctattcgctatagccggagaattggtaacctgtacacaggcagcttgtaccttggtcttacaagcttgtt
ggaaaactctaaaagtttacaaccgggagatcggatcggcctcttttcctatggcagtggtgcggtgtccgagttctttaccgggtatttagaagaa
aattaccaagagtacctgttcgctcaaagccatcaagaaatgctggatagccggactcggattacggtcgatgaatacgagaccatcttttcagag
actctgccagaacatggtgaatgcgccgaatatacgagcgacgtccccttttctataaccaagattgagaacgacattcgttattataaaatctga

Exemplary mvaS Polypeptides and Nucleic Acids

The mvaE nucleic acid can be expressed in a recombinant cell on a multicopy plasmid. The plasmid can be a high copy plasmid, a low copy plasmid, or a medium copy plasmid. Alternatively, the mvaE nucleic acid can be integrated into the host cell's chromosome. For both heterologous expression of an mvaE nucleic acid on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the mvaE nucleic acid.

The mvaS gene encodes a polypeptide that possesses HMG-CoA synthase activity. This polypeptide can convert acetoacetyl CoA to 3-hydroxy-3-methylglutaryl-CoA (HMG-CoA). Exemplary mvaS polypeptides and nucleic acids include naturally-occurring polypeptides and nucleic acids from any of the source organisms described herein as well as mutant polypeptides and nucleic acids derived from any of the source organisms described herein that have at least one activity of a mvaS polypeptide.

Mutant mvaS polypeptides include those in which one or more amino acid residues have undergone an amino acid substitution while retaining mvaS polypeptide activity (i.e., the ability to convert acetoacetyl CoA to 3-hydroxy-3-methylglutaryl-CoA). Amino acid substitutions in the mvaS polypeptide can be introduced to improve the functionality of the molecule. For example, amino acid substitutions that increase the binding affinity of the mvaS polypeptide for its substrate, or that improve its ability to convert acetoacetyl CoA to 3-hydroxy-3-methylglutaryl-CoA can be introduced into the mvaS polypeptide. In some aspects, the mutant mvaS polypeptides contain one or more conservative amino acid substitutions.

Standard methods, such as those described in Quant et al. (Biochem J., 1989, 262:159-164), can be used to determine whether a polypeptide has mvaS activity, by measuring HMG-CoA synthase activity. In an exemplary assay, HMG-CoA synthase activity can be assayed by spectrophotometrically measuring the disappearance of the enol form of acetoacetyl-CoA by monitoring the change of absorbance at 303 nm. A standard 1 ml assay system containing 50 mm-Tris/HCl, pH 8.0, 10 mM-MgCl2 and 0.2 mM-dithiothreitol at 30° C.; 5 mM-acetyl phosphate, 10,M-acetoacetyl-CoA and 5 μl samples of extracts can be added, followed by simultaneous addition of acetyl-CoA (100 μM) and 10 units of PTA. HMG-CoA synthase activity is then measured as the difference in the rate before and after acetyl-CoA addition. The absorption coefficient of acetoacetyl-CoA under the conditions used (pH 8.0, 10 mM-MgCl₂), is 12.2×10³ M⁻¹ cm⁻¹. By definition, 1 unit of enzyme activity causes 1 μmol of acetoacetyl-CoA to be transformed per minute.

Exemplary mvaS nucleic acids include nucleic acids that encode a polypeptide, fragment of a polypeptide, peptide, or fusion polypeptide that has at least one activity of a mvaS polypeptide. Exemplary mvaS polypeptides and nucleic acids include naturally-occurring polypeptides and nucleic acids from any of the source organisms described herein as well as mutant polypeptides and nucleic acids derived from any of the source organisms described herein. Exemplary mvaS nucleic acids include, for example, mvaS nucleic acids isolated from Listeria grayi_DSM 20601, Enterococcus faecium, Enterococcus gallinarum EG2, Enterococcus faecalis, and/or Enterococcus casseliflavus. The mvaS nucleic acid encoded by the Listeria grayi_DSM 20601 mvaS gene can have a 99%, 98%, 97%, 96%, 95%, 95%, 93%, 92%, 91%, 90%, 89%, 88%, 87%, 86%, or 85% sequence identity to SEQ ID NO: 7. The mvaS nucleic acid encoded by the Enterococcus faecium mvaS gene can have a 99%, 98%, 97%, 96%, 95%, 95%, 93%, 92%, 91%, 90%, 89%, 88%, 87%, 86%, or 85% sequence identity to SEQ ID NO:8. The mvaS nucleic acid encoded by the Enterococcus gallinarum EG2 mvaS gene can have a 99%, 98%, 97%, 96%, 95%, 95%, 93%, 92%, 91%, 90%, 89%, 88%, 87%, 86%, or 85% sequence identity to SEQ ID NO:9. The mvaS nucleic acid encoded by the Enterococcus casseliflavus mvaS gene can have a 99%, 98%, 97%, 96%, 95%, 95%, 93%, 92%, 91%, 90%, 89%, 88%, 87%, 86%, or 85% sequence identity to SEQ ID NO:10. The mvaS nucleic acid encoded by the Enterococcus faecalis mvaS gene can have a 99%, 98%, 97%, 96%, 95%, 95%, 93%, 92%, 91%, 90%, 89%, 88%, 87%, 86%, or 85% sequence identity to the mvaE gene previously disclosed in E. coli to produce mevalonate (see US 2005/0287655 A1; Tabata, K. and Hashimoto, S.-I. Biotechnology Letters 26: 1487-1491, 2004).

Sequence of Listeria grayi DSM 20601 mvaS
SEQ ID NO: 7
atggaagaagtggtaattatagatgcacgtcggactccgattggtaaatatcacgggtcgttgaagaagttttcagcggtggcgctggggacggc
cgtggctaaagacatgttcgaacgcaaccagaaaatcaaagaggagatcgcgcaggtcataattggtaatgtcttgcaggcaggaaatggcca
gaaccccgcgcggcaagttgctcttcaatcagggttgtccgttgacattcccgcttctacaattaacgaggtttgtgggtctggtttgaaagctatctt
gatgggcatggaacaaatccaactcggcaaagcgcaagtagtgctggcaggcggcattgaatcaatgacaaatgcgccaagcctgtcccacta
taacaaggcggaggatacgtatagtgtcccagtgtcgagcatgacactggatggtctgacagacgcattttctagtaaacctatgggattaacagc
ggaaaacgtcgcacagcgctacggtatctcccgtgaggcgcaagatcaattcgcatatcaatctcagatgaaagcagcaaaagcgcaggcag
aaaacaaattcgctaaggaaattgtgccactggcgggtgaaactaaaaccatcacagctgacgaagggatcagatcccaaacaacgatggaga
aactggcaagtctcaaacctgtttttaaaaccgatggcactgtaaccgcagggaatgctagcaccattaatgacggggccgcccttgtgctgcttg
ctagcaaaacttactgcgaaactaatgacataccgtaccttgcgacaatcaaagaaattgttgaagttggaatcgatccggagattatgggcatctc
tccgataaaagcgatacaaacattgttacaaaatcaaaaagttagcctcgaagatattggagtttttgaaataaatgaagcctttgccgcaagtagc
atagtggttgaatctgagttgggattagatccggctaaagttaaccgttatgggggtggtatatccttaggtcatgcaattggggcaaccggcgctc
gcctggccacttcactggtgtatcaaatgcaggagatacaagcacgttatggtattgcgagcctgtgcgttggtggtggacttggactggcaatgc
ttttagaacgtccaactattgagaaggctaaaccgacagacaaaaagttctatgaattgtcaccagctgaacggttgcaagagctggaaaatcaac
agaaaatcagttctgaaactaaacagcagttatctcagatgatgcttgccgaggacactgcaaaccatttgatagaaaatcaaatatcagagattga
actcccaatgggcgtcgggatgaacctgaaggttgatgggaaagcctatgttgtgccaatggcgacggaagagccgtccgtcatcgcggccat
gtctaatggtgccaaaatggccggcgaaattcacactcagtcgaaagaacggctgctcagaggtcagattgttttcagcgcgaagaatccgaat
gaaatcgaacagagaatagctgagaaccaagctttgattttcgaacgtgccgaacagtcctatccttccattgtgaaaagagagggaggtctccg
ccgcattgcacttcgtcattttcctgccgattctcagcaggagtctgcggaccagtccacatttttatcagtggacctttttgtagatgtgaaagacgc
gatgggggcaaatatcataaatgcaatacttgagggcgtcgcagccctgtttcgcgaatggttccccaatgaggaaattcttttttctattctctcgaa
cttggctacggagagcttagtcacggctgtttgtgaagtcccatttagtgcacttagcaagagaggtggtgcaacggtggcccagaaaattgtgc
aggcgtcgctcttcgcaaagacagacccataccgcgcagtgacccacaacaaagggattatgaacggtgtagaggctgttatgcttgccacag
gcaacgacacgcgcgcagtctcagccgcttgtcatggatacgcagcgcgcaccggtagctatcagggtctgactaactggacgattgagtcgg
atcgcctggtaggcgagataacactgccgctggccatcgctacagttggaggcgctaccaaagtgttgcccaaagctcaagcggcactggaga
ttagtgatgttcactcttctcaagagcttgcagccttagcggcgtcagtaggtttagtacaaaatctcgcggccctgcgcgcactggtttccgaagg
tatacaaaaagggcacatgtccatgcaagcccggtctctcgcaatcgcggtcggtgctgaaaaagccgagatcgagcaggtcgccgaaaagtt
gcggcagaacccgccaatgaatcagcagcaggcgctccgttttcttggcgagatccgcgaacaatga
Sequence of Enterococcus faecium mvaS
SEQ ID NO: 8
atgaacgtcggcattgacaaaattaattttttcgttccaccgtattatctggatatggtcgacctggcccacgcacgcgaagtggacccgaacaaat
ttacaattggaattggacaggatcagatggctgtgagcaaaaagacgcacgatatcgtaacattcgcggctagtgccgcgaaggaaattttagaa
cctgaggacttgcaagctatagacatggttatagttggtaccgaatcgggcattgacgagagcaaagcatccgcggtcgttttacatcgtttgttgg
gcgtacaacctttcgctcgcagttttgaaattaaagaagcctgttacggggcaaccgcaggcattcagtttgccaagactcatatacaagcgaacc
cggagagcaaggtcctggtaattgcaagcgatatagctcggtatggtcttcggtcaggtggagagcccacacaaggcgcaggggcagttgcta
tgcttctcacggcaaatcccagaatcctgaccttcgaaaacgacaatctgatgttaacgcaggatatttatgacttctggagaccacttggtcacgct
taccctatggtagatggccacctttccaatcaagtctatattgacagttttaagaaggtctggcaagcacattgcgaacgcaatcaagcttctatatc
cgactatgccgcgattagttttcatattccgtatacaaaaatgggtaagaaagccctgctcgctgtttttgcagatgaagtggaaactgaacaggaa
cgcgttatggcacggtatgaagagtctatcgtatattcacgccggatcggcaacttgtatacgggatcattgtacctggggctgatatccttattgga
aaacagttctcacctgtcggcgggcgaccggataggattgtttagttatgggagtggcgctgtcagcgaatttttctccggtcgtttagtggcaggc
tatgaaaatcaattgaacaaagaggcgcatacccagctcctggatcagcgtcagaagctttccatcgaagagtatgaggcgatttttacagattcct
tagaaattgatcaggatgcagcgttctcggatgacctgccatattccatccgcgagataaaaaacacgattcggtactataaggagagctga
Sequence of Enterococcus gallinarum EG2 mvaS
SEQ ID NO: 9
atggaagaagttgtcatcattgacgcactgcgtactccaataggaaagtaccacggttcgctgaaagattacacagctgttgaactggggacagt
agcagcaaaggcgttgctggcacgaaatcagcaagcaaaagaacacatagcgcaagttattattggcaacgtcctgcaagccggaagtgggc
agaatccaggccgacaagtcagtttacagtcaggattgtcttctgatatccccgctagcacgatcaatgaagtgtgtggctcgggtatgaaagcga
ttctgatgggtatggagcaaattcagctgaacaaagcctctgtggtcttaacaggcggaattgaaagcatgaccaacgcgccgctgtttagttatta
caacaaggctgaggatcaatattcggcgccggttagcacaatgatgcacgatggtctaacagatgctttcagttccaaaccaatgggcttaaccg
cagagaccgtcgctgagagatatggaattacgcgtaaggaacaagatgaatttgcttatcactctcaaatgaaggcggccaaagcccaggcgg
cgaaaaagtttgatcaggaaattgtacccctgacggaaaaatccggaacggttctccaggacgaaggcatcagagccgcgacaacagtcgag
aagctagctgagcttaaaacggtgttcaaaaaagacggaacagttacagcgggtaacgcctctacgataaatgatggcgctgctatggtattaat
agcatcaaaatcttattgcgaagaacaccagattccttatctggccgttataaaggagatcgttgaggtgggttttgcccccgaaataatgggtattt
cccccattaaggctatagacaccctgctgaaaaatcaagcactgaccatagaggatataggaatatttgagattaatgaagcctttgctgcgagttc
gattgtggtagaacgcgagttgggcctggaccccaaaaaagttaatcgctatggcggtggtatatcactcggccacgcaattggggcgacggg
agctcgcattgcgacgaccgttgcttatcagctgaaagatacccaggagcgctacggtatagcttccttatgcgttggtgggggtcttggattggc
gatgcttctggaaaacccatcggccactgcctcacaaactaattttgatgaggaatctgcttccgaaaaaactgagaagaagaagttttatgcgcta
gctcctaacgaacgcttagcgtttttggaagcccaaggcgctattaccgctgctgaaaccctggtcttccaggagatgaccttaaacaaagagac
agccaatcacttaatcgaaaaccaaatcagcgaagttgaaattcctttaggcgtgggcctgaacttacaggtgaatgggaaagcgtataatgttcc
tctggccacggaggaaccgtccgttatcgctgcgatgtcgaatggcgccaaaatggctggtcctattacaacaacaagtcaggagaggctgtta
cggggtcagattgtcttcatggacgtacaggacccagaagcaatattagcgaaagttgaatccgagcaagctaccattttcgcggtggcaaatga
aacatacccgtctatcgtgaaaagaggaggaggtctgcgtagagtcattggcaggaatttcagtccggccgaaagtgacttagccacggcgtat
gtatcaattgacctgatggtagatgttaaggatgcaatgggtgctaatatcatcaatagtatcctagaaggtgttgcggaattgtttagaaaatggttc
ccagaagaagaaatcctgttctcaattctctccaatctcgcgacagaaagtctggtaacggcgacgtgctcagttccgtttgataaattgtccaaaa
ctgggaatggtcgacaagtagctggtaaaatagtgcacgcggcggactttgctaagatagatccatacagagctgccacacacaataaaggtatt
atgaatggcgttgaagcgttaatcttagccaccggtaatgacacccgtgcggtgtcggctgcatgccacggttacgcggcacgcaatgggcgaa
tgcaagggcttacctcttggacgattatcgaagatcggctgataggctctatcacattacctttggctattgcgacagtggggggtgccacaaaaat
cttgccaaaagcacaggccgccctggcgctaactggcgttgagacggcgtcggaactggccagcctggcggcgagtgtgggattagttcaaa
atttggccgctttacgagcactagtgagcgagggcattcagcaagggcacatgagtatgcaagctagatccctggccattagcgtaggtgcgaa
aggtactgaaatagagcaactagctgcgaagctgagggcagcgacgcaaatgaatcaggagcaggctcgtaaatttctgaccgaaataagaaa
ttaa
Sequence of Enterococcus casseliflavus mvaS
SEQ ID NO: 10
atgaacgttggaattgataaaatcaattttttcgttccgccctatttcattgatatggtggatctcgctcatgcaagagaagttgaccccaacaagttca
ctataggaataggccaagatcagatggcagtaaacaagaaaacgcaagatatcgtaacgttcgcgatgcacgccgcgaaggatattctgactaa
ggaagatttacaggccatagatatggtaatagtggggactgagtctgggatcgacgagagcaaggcaagtgctgtcgtattgcatcggcttttag
gtattcagccttttgcgcgctcctttgaaattaaggaggcatgctatggggccactgccggccttcagtttgcaaaagctcatgtgcaggctaatcc
ccagagcaaggtcctggtggtagcttccgatatagcacgctacggactggcatccggaggagaaccgactcaaggtgtaggtgctgtggcaat
gttgatttccgctgatccagctatcttgcagttagaaaatgataatctcatgttgacccaagatatatacgatttttggcgcccggtcgggcatcaatat
cctatggtagacggccatctgtctaatgccgtctatatagacagctttaaacaagtctggcaagcacattgcgagaaaaaccaacggactgctaaa
gattatgctgcattgtcgttccatattccgtacacgaaaatgggtaagaaagctctgttagcggtttttgcggaggaagatgagacagaacaaaag
cggttaatggcacgttatgaagaatcaattgtatacagtcgtcggactggaaatctgtatactggctcactctatctgggcctgatttccttactggag
aatagtagcagtttacaggcgaacgatcgcataggtctgtttagctatggttcaggggccgttgcggaatttttcagtggcctcttggtaccgggtta
cgagaaacaattagcgcaagctgcccatcaagctcttctggacgaccggcaaaaactgactatcgcagagtacgaagccatgtttaatgaaacc
attgatattgatcaggaccagtcatttgaggatgacttactgtactccatcagagagatcaaaaacactattcgctactataacgaggagaatgaata
a

The mvaS nucleic acid can be expressed in a recombinant cell on a multicopy plasmid. The plasmid can be a high copy plasmid, a low copy plasmid, or a medium copy plasmid. Alternatively, the mvaS nucleic acid can be integrated into the host cell's chromosome. For both heterologous expression of an mvaS nucleic acid on a plasmid or as an integrated part of the host cell's chromosome, expression of the nucleic acid can be driven by either an inducible promoter or a constitutively expressing promoter. The promoter can be a strong driver of expression, it can be a weak driver of expression, or it can be a medium driver of expression of the mvaS nucleic acid.

Compositions of recombinant cells as described herein are contemplated within the scope of the invention as well. It is understood that recombinant cells also encompass progeny cells as well.

### Nucleic Acids Encoding Polypeptides of the Lower MVA Pathway

In some aspects of the invention, the cells described in any of the compositions or methods described herein further comprise one or more nucleic acids encoding a lower mevalonate (MVA) pathway polypeptide(s). In some aspects, the lower MVA pathway polypeptide is an endogenous polypeptide. In some aspects, the endogenous nucleic acid encoding a lower MVA pathway polypeptide is operably linked to a constitutive promoter. In some aspects, the endogenous nucleic acid encoding a lower MVA pathway polypeptide is operably linked to an inducible promoter. In some aspects, the endogenous nucleic acid encoding a lower MVA pathway polypeptide is operably linked to a strong promoter. In a particular aspect, the cells are engineered to over-express the endogenous lower MVA pathway polypeptide relative to wild-type cells. In some aspects, the endogenous nucleic acid encoding a lower MVA pathway polypeptide is operably linked to a weak promoter.

The lower mevalonate biosynthetic pathway comprises mevalonate kinase (MVK), phosphomevalonate kinase (PMK), and diphosphomevalonte decarboxylase (MVD). In some aspects, the lower MVA pathway can further comprise isopentenyl diphosphate isomerase (IDI). Cells provided herein can comprise at least one nucleic acid encoding isoprene synthase, one or more upper MVA pathway polypeptides, and/or one or more lower MVA pathway polypeptides. Polypeptides of the lower MVA pathway can be any enzyme (a) that phosphorylates mevalonate to mevalonate 5-phosphate; (b) that converts mevalonate 5-phosphate to mevalonate 5-pyrophosphate; and (c) that converts mevalonate 5-pyrophosphate to isopentenyl pyrophosphate. More particularly, the enzyme that phosphorylates mevalonate to mevalonate 5-phosphate can be from the group consisting of M. mazei mevalonate kinase, Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, and Methanococcoides burtonii mevalonate kinase polypeptide. In another aspect, the enzyme that phosphorylates mevalonate to mevalonate 5-phosphate is M. mazei mevalonate kinase. In yet another aspect, the enzyme that phosphorylates mevalonate to mevalonate 5-phosphate is M. burtonii mevalonate kinase.

In some aspects, the lower MVA pathway polypeptide is a heterologous polypeptide. In some aspects, the cells comprise more than one copy of a heterologous nucleic acid encoding a lower MVA pathway polypeptide. In some aspects, the heterologous nucleic acid encoding a lower MVA pathway polypeptide is operably linked to a constitutive promoter. In some aspects, the heterologous nucleic acid encoding a lower MVA pathway polypeptide is operably linked to an inducible promoter. In some aspects, the heterologous nucleic acid encoding a lower MVA pathway polypeptide is operably linked to a strong promoter. In some aspects, the heterologous nucleic acid encoding a lower MVA pathway polypeptide is operably linked to a weak promoter. In some aspects, the heterologous lower MVA pathway polypeptide is a polypeptide from Saccharomyces cerevisiae, Enterococcus faecalis, Methanosarcina mazei, or Methanococcoides burtonii.

The nucleic acids encoding a lower MVA pathway polypeptide(s) can be integrated into a genome of the cells or can be stably expressed in the cells. The nucleic acids encoding a lower MVA pathway polypeptide(s) can additionally be on a vector.

Exemplary lower MVA pathway polypeptides are also provided below: (i) mevalonate kinase (MVK); (ii) phosphomevalonate kinase (PMK); (iii) diphosphomevalonate decarboxylase (MVD); and (iv) isopentenyl diphosphate isomerase (IDI). In particular, the lower MVK polypeptide can be from the genus Methanosarcina and, more specifically, the lower MVK polypeptide can be from Methanosarcina mazei. Additional examples of lower MVA pathway polypeptides can be found in U.S. Patent Application Publication 2010/0086978 the contents of which are expressly incorporated herein by reference in their entirety with respect to lower MVK pathway polypeptides and lower MVK pathway polypeptide variants.

Any one of the cells described herein can comprise IDI nucleic acid(s) (e.g., endogenous or heterologous nucleic acid(s) encoding IDI). Isopentenyl diphosphate isomerase polypeptides (isopentenyl-diphosphate delta-isomerase or IDI) catalyzes the interconversion of isopentenyl diphosphate (IPP) and dimethylallyl diphosphate (DMAPP) (e.g., converting IPP into DMAPP and/or converting DMAPP into IPP). Exemplary IDI polypeptides include polypeptides, fragments of polypeptides, peptides, and fusions polypeptides that have at least one activity of an IDI polypeptide. Standard methods (such as those described herein) can be used to determine whether a polypeptide has IDI polypeptide activity by measuring the ability of the polypeptide to interconvert IPP and DMAPP in vitro, in a cell extract, or in vivo. Exemplary IDI nucleic acids include nucleic acids that encode a polypeptide, fragment of a polypeptide, peptide, or fusion polypeptide that has at least one activity of an IDI polypeptide. Exemplary IDI polypeptides and nucleic acids include naturally-occurring polypeptides and nucleic acids from any of the source organisms described herein as well as mutant polypeptides and nucleic acids derived from any of the source organisms described herein.

Lower MVA pathway polypeptides include polypeptides, fragments of polypeptides, peptides, and fusions polypeptides that have at least one activity of a lower MVA pathway polypeptide. Exemplary lower MVA pathway nucleic acids include nucleic acids that encode a polypeptide, fragment of a polypeptide, peptide, or fusion polypeptide that has at least one activity of a lower MVA pathway polypeptide. Exemplary lower MVA pathway polypeptides and nucleic acids include naturally-occurring polypeptides and nucleic acids from any of the source organisms described herein. In addition, variants of lower MVA pathway polypeptides that confer the result of better isoprene production can also be used as well.

In some aspects, the lower MVA pathway polypeptide is a polypeptide from Saccharomyces cerevisiae, Enterococcus faecalis, Methanosarcina mazei, or Methanococcoides burtonii. In some aspects, the MVK polypeptide is selected from the group consisting of Lactobacillus mevalonate kinase polypeptide, Lactobacillus sakei mevalonate kinase polypeptide, yeast mevalonate kinase polypeptide, Saccharomyces cerevisiae mevalonate kinase polypeptide, Streptococcus mevalonate kinase polypeptide, Streptococcus pneumoniae mevalonate kinase polypeptide, Streptomyces mevalonate kinase polypeptide, Streptomyces CL190 mevalonate kinase polypeptide, Methanosarcina mazei mevalonate kinase polypeptide, and Methanococcoides burtonii mevalonate kinase polypeptide. Any one of the promoters described herein (e.g., promoters described herein and identified in the Examples of the present disclosure including inducible promoters and constitutive promoters) can be used to drive expression of any of the MVA polypeptides described herein.

### DXP Pathway Nucleic Acids and Polypeptides

In some aspects of the invention, the recombinant cells described in any of the compositions or methods described herein further comprise one or more heterologous nucleic acids encoding a DXS polypeptide or other DXP pathway polypeptides. In some aspects, the cells further comprise a chromosomal copy of an endogenous nucleic acid encoding a DXS polypeptide or other DXP pathway polypeptides. In some aspects, the E. coli cells further comprise one or more nucleic acids encoding an IDI polypeptide and a DXS polypeptide or other DXP pathway polypeptides. In some aspects, one nucleic acid encodes the isoprene synthase polypeptide, IDI polypeptide, and DXS polypeptide or other DXP pathway polypeptides. In some aspects, one plasmid encodes the isoprene synthase polypeptide, IDI polypeptide, and DXS polypeptide or other DXP pathway polypeptides. In some aspects, multiple plasmids encode the isoprene synthase polypeptide, IDI polypeptide, and DXS polypeptide or other DXP pathway polypeptides.

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS polypeptides and nucleic acids and methods of measuring DXS activity are described in more detail in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716, the contents of which are expressly incorporated herein by reference in their entirety with respect to DXP pathway polypeptides.

Exemplary DXP pathways polypeptides include, but are not limited to any of the following polypeptides: DXS polypeptides, DXR polypeptides, MCT polypeptides, CMK polypeptides, MCS polypeptides, HDS polypeptides, HDR polypeptides, and polypeptides (e.g., fusion polypeptides) having an activity of one, two, or more of the DXP pathway polypeptides. In particular, DXP pathway polypeptides include polypeptides, fragments of polypeptides, peptides, and fusions polypeptides that have at least one activity of a DXP pathway polypeptide. Exemplary DXP pathway nucleic acids include nucleic acids that encode a polypeptide, fragment of a polypeptide, peptide, or fusion polypeptide that has at least one activity of a DXP pathway polypeptide. Exemplary DXP pathway polypeptides and nucleic acids include naturally-occurring polypeptides and nucleic acids from any of the source organisms described herein as well as mutant polypeptides and nucleic acids derived from any of the source organisms described herein. Exemplary DXP pathway polypeptides and nucleic acids and methods of measuring DXP pathway polypeptide activity are described in more detail in International Publication No.: WO 2010/148150

Exemplary DXS polypeptides include polypeptides, fragments of polypeptides, peptides, and fusions polypeptides that have at least one activity of a DXS polypeptide. Standard methods (such as those described herein) can be used to determine whether a polypeptide has DXS polypeptide activity by measuring the ability of the polypeptide to convert pyruvate and D-glyceraldehyde-3-phosphate into 1-deoxy-D-xylulose-5-phosphate in vitro, in a cell extract, or in vivo. Exemplary DXS polypeptides and nucleic acids and methods of measuring DXS activity are described in more detail in International Publication No. WO 2009/076676, U.S. patent application Ser. No. 12/335,071 (US Publ. No. 2009/0203102), WO 2010/003007, US Publ. No. 2010/0048964, WO 2009/132220, and US Publ. No. 2010/0003716.

In particular, DXS polypeptides convert pyruvate and D-glyceraldehyde 3-phosphate into 1-deoxy-d-xylulose 5-phosphate (DXP). Standard methods can be used to determine whether a polypeptide has DXS polypeptide activity by measuring the ability of the polypeptide to convert pyruvate and D-glyceraldehyde 3-phosphate in vitro, in a cell extract, or in vivo.

DXR polypeptides convert 1-deoxy-d-xylulose 5-phosphate (DXP) into 2-C-methyl-D-erythritol 4-phosphate (MEP). Standard methods can be used to determine whether a polypeptide has DXR polypeptides activity by measuring the ability of the polypeptide to convert DXP in vitro, in a cell extract, or in vivo.

MCT polypeptides convert 2-C-methyl-D-erythritol 4-phosphate (MEP) into 4-(cytidine 5′-diphospho)-2-methyl-D-erythritol (CDP-ME). Standard methods can be used to determine whether a polypeptide has MCT polypeptides activity by measuring the ability of the polypeptide to convert MEP in vitro, in a cell extract, or in vivo.

CMK polypeptides convert 4-(cytidine 5′-diphospho)-2-C-methyl-D-erythritol (CDP-ME) into 2-phospho-4-(cytidine 5′-diphospho)-2-C-methyl-D-erythritol (CDP-MEP). Standard methods can be used to determine whether a polypeptide has CMK polypeptides activity by measuring the ability of the polypeptide to convert CDP-ME in vitro, in a cell extract, or in vivo.

MCS polypeptides convert 2-phospho-4-(cytidine 5′-diphospho)-2-C-methyl-D-erythritol (CDP-MEP) into 2-C-methyl-D-erythritol 2,4-cyclodiphosphate (ME-CPP or cMEPP). Standard methods can be used to determine whether a polypeptide has MCS polypeptides activity by measuring the ability of the polypeptide to convert CDP-MEP in vitro, in a cell extract, or in vivo.

HDS polypeptides convert 2-C-methyl-D-erythritol 2,4-cyclodiphosphate into (E)-4-hydroxy-3-methylbut-2-en-1-yl diphosphate (HMBPP or HDMAPP). Standard methods can be used to determine whether a polypeptide has HDS polypeptides activity by measuring the ability of the polypeptide to convert ME-CPP in vitro, in a cell extract, or in vivo.

HDR polypeptides convert (E)-4-hydroxy-3-methylbut-2-en-1-yl diphosphate into isopentenyl diphosphate (IPP) and dimethylallyl diphosphate (DMAPP). Standard methods can be used to determine whether a polypeptide has HDR polypeptides activity by measuring the ability of the polypeptide to convert HMBPP in vitro, in a cell extract, or in vivo.

### Source Organisms for MVA Pathway, Isoprene Synthase, IDI, and DXP Pathway Polypeptides

Isoprene synthase, IDI, DXP pathway, and/or MVA pathway nucleic acids can be obtained from any organism that naturally contains isoprene synthase, IDI, DXP pathway, and/or MVA pathway nucleic acids. Isoprene is formed naturally by a variety of organisms, such as bacteria, yeast, plants, and animals. Some organisms contain the MVA pathway for producing isoprene. Isoprene synthase nucleic acids can be obtained, e.g., from any organism that contains an isoprene synthase. MVA pathway nucleic acids can be obtained, e.g., from any organism that contains the MVA pathway. IDI and DXP pathway nucleic acids can be obtained, e.g., from any organism that contains the IDI and DXP pathway.

The nucleic acid sequence of the isoprene synthase, DXP pathway, IDI, and/or MVA pathway nucleic acids can be isolated from a bacterium, fungus, plant, algae, or cyanobacterium. Exemplary source organisms include, for example, yeasts, such as species of Saccharomyces (e.g., S. cerevisiae), bacteria, such as species of Escherichia (e.g., E. coli), species of Methanosarcina (e.g., Methanosarcina mazei) or species of Methanococcoides (e.g., Methanococcoides burtonii), plants, such as kudzu or poplar (e.g., Populus alba or Populus alba×tremula CAC35696) or aspen (e.g., Populus tremuloides). Exemplary sources for isoprene synthases, IDI, and/or MVA pathway polypeptides which can be used are also described in International Patent Application Publication Nos. WO2009/076676, WO2010/003007, WO2009/132220, WO2010/031062, WO2010/031068, WO2010/031076, WO2010/013077, WO2010/031079, WO2010/148150, WO2010/078457, and WO2010/148256.

In some aspects, the source organism is a yeast, such as Saccharomyces sp., Schizosaccharomyces sp., Pichia sp., or Candida sp.

In some aspects, the source organism is a bacterium, such as strains of Bacillus such as B. lichenformis or B. subtilis, strains of Pantoea such as P. citrea, strains of Pseudomonas such as P. alcaligenes, strains of Streptomyces such as S. lividans or S. rubiginosus, strains of Escherichia such as E. coli, strains of Enterobacter, strains of Streptococcus, or strains of Archaea such as Methanosarcina mazei.

As used herein, “the genus Bacillus” includes all species within the genus “Bacillus,” as known to those of skill in the art, including but not limited to B. subtilis, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, and B. thuringiensis. It is recognized that the genus Bacillus continues to undergo taxonomical reorganization. Thus, it is intended that the genus include species that have been reclassified, including but not limited to such organisms as B. stearothermophilus, which is now named “Geobacillus stearothermophilus.” The production of resistant endospores in the presence of oxygen is considered the defining feature of the genus Bacillus, although this characteristic also applies to the recently named Alicyclobacillus, Amphibacillus, Aneurinibacillus, Anoxybacillus, Brevibacillus, Filobacillus, Gracilibacillus, Halobacillus, Paenibacillus, Salibacillus, Thermobacillus, Ureibacillus, and Virgibacillus.

In some aspects, the source organism is a gram-positive bacterium. Non-limiting examples include strains of Streptomyces (e.g., S. lividans, S. coelicolor, or S. griseus) and Bacillus. In some aspects, the source organism is a gram-negative bacterium, such as E. coli or Pseudomonas sp. In some aspects, the source organism is L. acidophilus.

In some aspects, the source organism is a plant, such as a plant from the family Fabaceae, such as the Faboideae subfamily. In some aspects, the source organism is kudzu, poplar (such as Populus alba×tremula CAC35696), aspen (such as Populus tremuloides), or Quercus robur.

In some aspects, the source organism is an algae, such as a green algae, red algae, glaucophytes, chlorarachniophytes, euglenids, chromista, or dinoflagellates.

In some aspects, the source organism is a cyanobacteria, such as cyanobacteria classified into any of the following groups based on morphology: Chroococcales, Pleurocapsales, Oscillatoriales, Nostocales, or Stigonematales.

### Recombinant Cells Capable of Increased Production of Isoprene

The recombinant marine bacterial cells described herein have the ability to produce isoprene at a concentration greater than that of the same cells lacking one or more heterologous nucleic acid encoding an isoprene synthase polypeptide when cultured under the same conditions. In one aspect, the cells can further comprise one or more heterologous nucleic acid encoding for one or more MVA pathway polypeptide and/or one or more DXP pathway polypeptide. In another aspect, the cells can further comprise one or more heterologous nucleic acid encoding for one or more MVA pathway polypeptide and/or one or more endogenous polynucleotide sequence encoding for one or more DXP pathway polypeptide. In yet another aspect, the cells can further comprise one or more heterologous polynucleotide encoding an IDI polypeptide. In some aspects, the recombinant marine bacterial cells comprise one or more copies of a heterologous nucleic acid encoding an isoprene synthase polypeptide isolated from the family Fabaceae, the family Salicaceae, or the family Fagaceae. In certain aspects, the recombinant marine bacterial cells comprise one or more copies of a heterologous nucleic acid encoding an isoprene synthase polypeptide isolated from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), or English Oak (Quercus robur) (Zimmer et al., WO 98/02550) or comprise a variant of the same. In other aspects, the recombinant marine bacterial cells comprise one or more copies of a heterologous nucleic acid encoding an isoprene synthase polypeptide isolated from Pueraria montana, Pueraria lobata, Populus tremuloides, Populus alba, Populus nigra, or Populus trichocarpa or comprise a variant of the same. In further aspects, the recombinant marine bacterial cells comprise one or more copies of a heterologous nucleic acid encoding an isoprene synthase polypeptide isolated from Populus alba or comprise a variant of the same. In certain aspects, the recombinant marine bacterial cells comprise one or more copies of a heterologous nucleic acid encoding an isoprene synthase polypeptide isolated from wherein the polynucleotide sequence encoding the isoprene synthase (e.g., isoprene synthase from Populus alba or a variant thereof) is codon optimized. In some aspects, the one or more copies of a heterologous nucleic acid encoding isoprene synthase are heterologous nucleic acids that are integrated into the host cell's chromosomal nucleotide sequence. In other aspects, the one or more heterologous nucleic acids are integrated into plasmid. In still other aspects, at least one of the one or more heterologous nucleic acids is integrated into the cell's chromosomal nucleotide sequence while at least one of the one or more heterologous nucleic acid sequences is integrated into a plasmid. The recombinant cells can produce at least 5% greater amounts of isoprene compared to isoprene-producing cells that do not comprise the isoprene synthase polypeptide. Alternatively, the recombinant cells can produce greater than about 1%, 2%, 3%, 4%, 5%, 6%, 7%, 8%, 9%, 10%, 11%, 12%, 13%, 14%, or 15% of isoprene, inclusive, as well as any numerical value in between these numbers.

The recombinant marine bacterial cells described herein have the ability to produce isoprene at a concentration greater than that of the same cells lacking one or more heterologous nucleic acid encoding an isoprene synthase polypeptide when cultured under the same conditions. In one aspect, the cells can further comprise one or more heterologous nucleic acid encoding for one or more MVA pathway polypeptide and/or one or more DXP pathway polypeptide. In another aspect, the cells can further comprise one or more heterologous nucleic acid encoding for one or more MVA pathway polypeptide and/or one or more endogenous polynucleotide sequence encoding for one or more DXP pathway polypeptide. In yet another aspect, the cells can further comprise one or more heterologous polynucleotide encoding an IDI polypeptide. In some aspects, the recombinant marine bacterial cells comprise one or more copies of a heterologous nucleic acid encoding an isoprene synthase polypeptide isolated from the family Fabaceae, the family Salicaceae, or the family Fagaceae. In certain aspects, the recombinant marine bacterial cells comprise one or more copies of a heterologous nucleic acid encoding an isoprene synthase polypeptide isolated from Pueraria montana (kudzu) (Sharkey et al., Plant Physiology 137: 700-712, 2005), Pueraria lobata, poplar (such as Populus alba, Populus nigra, Populus trichocarpa, or Populus alba×tremula (CAC35696) (Miller et al., Planta 213: 483-487, 2001), aspen (such as Populus tremuloides) (Silver et al., JBC 270(22): 13010-1316, 1995), or English Oak (Quercus robur) (Zimmer et al., WO 98/02550) or comprise a variant of the same. In other aspects, the recombinant marine bacterial cells comprise one or more copies of a heterologous nucleic acid encoding an isoprene synthase polypeptide isolated from Pueraria montana, Pueraria lobata, Populus tremuloides, Populus alba, Populus nigra, or Populus trichocarpa or comprise a variant of the same. In further aspects, the recombinant marine bacterial cells comprise one or more copies of a heterologous nucleic acid encoding an isoprene synthase polypeptide isolated from Populus alba or comprise a variant of the same. In certain aspects, the recombinant marine bacterial cells comprise one or more copies of a heterologous nucleic acid encoding an isoprene synthase polypeptide isolated from wherein the polynucleotide sequence encoding the isoprene synthase (e.g., isoprene synthase from Populus alba or a variant thereof) is codon optimized. Any of the one or more heterologous nucleic acids can be operably linked to constitutive promoters, can be operably linked to inducible promoters, or can be operably linked to a combination of inducible and constitutive promoters. The one or more heterologous nucleic acids can additionally be operably linked to strong promoters, weak promoters, and/or medium promoters. One or more of the heterologous nucleic acids encoding an isoprene synthase, a mevalonate (MVA) pathway polypeptide(s), and a DXP pathway polypeptide(s) can be integrated into a genome of the host cells or can be stably expressed in the cells. The one or more heterologous nucleic acids can additionally be on a vector.

One of skill in the art will recognize that expression vectors are designed to contain certain components which optimize gene expression for certain host marine bacterial strains. Such optimization components include, but are not limited to origin of replication, promoters, and enhancers. The vectors and components referenced herein are described for exemplary purposes and are not meant to narrow the scope of the invention.

The recombinant host cell according to any of the compositions or methods described is a marine bacterium. Any marine bacterium or progeny thereof can be used to express any of the genes (heterologous or endogenous) described herein. The marine bacterial cell can be classified as a proteobacteria. The marine bacterial cell can be classified as a gammaproteobacteria. The marine bacterial cell can be classified as a marine γ-proteobacterium. Marine bacterial cells, including gram positive or gram negative bacteria can be used to express any of the genes described herein. In particular, the genes described herein can be expressed in any one of, but not limited to, S. degradans 2-40, Alginovibrio aqualiticus, Alteromonas sr. strain KLIA, Asteromyces cruciatus, Beneckea pelagia, Corynebacterium spp., Enterobacter cloacae, Halmonas marina, Klebsiella pneumonia, Photobacterium spp. (ATCC 433367), Pseudoalteromonas elyakovii, Pseudomonas alginovora, Pseudomonas aeruginosa, Pseudomonas maltophilia, Pseudomonas putida, Vibrio alginolyticus, Vibrio halioticol, and Vibrio harveyi. In one embodiment, the marine bacterial cell is S. degradans 2-40 having the identifying characteristics of ATCC 43961. In another embodiment, the marine bacterial cell is Microbulbifer hydrolyticus IRE-31 having the identifying characteristics of ATCC 700072. In still another embodiment, the marine bacterial cell is Marinobacterium georgiense KW-40 having the identifying characteristics of ATCC 700074.

In aspects of the invention, the recombinant host cell can be a marine bacterium that is a cellulolytic bacterium. Such cellulolytic bacterial cells can be used to express any of the genes described herein. The marine cellulolytic bacterial cell can express a cellulolytic system that includes polypeptides with enzymatic activities. Enzymatic activities of a cellulolytic system include, but are not limited to, an endoglucanase, β-glucosidase, cellbiose or phosphorolase activity. In one embodiment, the cellulolytic bacterial cell is S. degradans 2-40. In a further embodiment, the marine cellulolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is an agarolytic bacterium. Such agarolytic bacterial cells can be used to express any of the genes described herein. The marine agarolytic bacterial cell can express an agarolytic system that includes polypeptides with enzymatic activities. Enzymatic activities of a cellulolytic system include, but are not limited to, an endoagarase, exoagarase, or neoagarbiose hydrolase activity. In one embodiment, the agarolytic bacterial cell is S. degradans 2-40. In a further embodiment, the marine agarolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is an alginolytic bacterium. Such alginolytic bacterial cells can be used to express any of the genes described herein. The marine alginolytic bacterial cell can express an alginolytic system that includes polypeptides with enzymatic activities. Enzymatic activities of a cellulolytic system include, but are not limited to, an alginate lyase, 4-deoxy-Lthreo-5-hexosulose uronate isomerase, 2-dehydro-3-deoxygluconate kinase, or 2-dehydro-3-deoxyphosphogluconatealdolase activity. In one embodiment, the alginolytic cell is S. degradans 2-40. In a further embodiment, the marine alginolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is a glucanolytic bacterium. Such glucanolytic bacterial cells can be used to express any of the genes described herein. The marine glucanolytic bacterial cell can express a glucanolytic system that includes polypeptides with enzymatic activities. Enzymatic activities of a glucanolytic system include, but are not limited to, an α-amylase, α-glucosidase, pullulananase, glucan 1,4-α-glucosidase, glucose kinase, sucrose phosphorylase, laminarinase, β-1,3(4)-endoglucanase, β-1,3-endoglucanase, β-1,6-glucosidase, or β-1,3-exoglucanase activity. In one embodiment, the glucanolytic cell is S. degradans 2-40. In a further embodiment, the marine glucanolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is a chitinolytic bacterium. Such chitinolytic bacterial cells can be used to express any of the genes described herein. The marine chitinolytic bacterial cell can express a chitinolytic system that includes polypeptides with enzymatic activities. Enzymatic activities of a chitinolytic system include, but are not limited to, an endochitinase, chitodextrinase, exochitinase, N-acetylglucaminidase, N-acetylglucosamine kinase, fructose 6P transmaminase, or N-acetylglucosamine deacetylase activity. In one embodiment, the chitinolytic cell is S. degradans 2-40. In a further embodiment, the marine chitinolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is a pectinolytic bacterium. Such pectinolytic bacterial cells can be used to express any of the genes described herein. The marine pectinolytic bacterial cell can express a pectinolytic system that includes polypeptides with enzymatic activities. Enzymatic activities of a pectinolytic system include, but are not limited to, a pectate lyase, rhamnogalacturon lyase, rhamnogalacturon hydrolase, pectinesterase, or exopectate lyase activity. In one embodiment, the pectinolytic cell is S. degradans 2-40. In a further embodiment, the marine pectinolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

In aspects of the invention, the recombinant host cell can be a marine bacterium that is a xylanolytic bacterium. Such xylanolytic bacterial cells can be used to express any of the genes described herein. The marine xylanolytic bacterial cell can express a xylanolytic system that includes polypeptides with enzymatic activities. Enzymatic activities of a xylanolytic system include, but are not limited to, an endoxylanase, xylosidase, α-galactosidase, α-glucuronidase, β-glucuronidase, arabinofuranosidase, arabitan endo 1,5-α-arabinosidase, arabinogalactan endo 1,4-β-galactosidase, acetoxylan esterase, carboxyl esterase, or β-galactosidase activity. In one embodiment, the xylanolytic cell is S. degradans 2-40. In a further embodiment, the marine xylanolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, or alginic acid.

In aspects of the invention, the recombinant host cell can be a marine bacterium that is a mannanolytic bacterium. Such mannanolytic bacterial cells can be used to express any of the genes described herein. The marine mannanolytic bacterial cell can express a mannanolytic system that includes polypeptides with enzymatic activities. Enzymatic activities of a mannanolytic system include, but are not limited to, a mannanase or mannosidase activity. In one embodiment, the mannanolytic cell is S. degradans 2-40. In a further embodiment, the marine mannanolytic bacterial cell can degrade biomass components (e.g., complex carbohydrates) including, but not limited to, cellulose, fucoidan, pectin, glucomannan, galactomannan, xylan, chitin, agar, laminarin, β-glucan, pullulan, starch, and alginic acid.

Additional examples of cellulolytic, agarolytic, alginolytic, glucanolytic, chitinolytic, pectinolytic, xylanolytic, mannanolytic bacterial characteristics can be found in Hutcheson S W, et al., (2011) Mar. Drugs, 9:645:665, the contents of which are expressly incorporated herein by reference in their entirety.

In another aspect of the invention, the recombinant host cell can be a bacterium that has high sequence similarity to a cellulolytic bacterium. Such bacterial cells can be used to express any of the genes described herein. In particular, the genes described herein can be expressed in any one of Cellvibrio mixtus, Cellvibrio japonicas, Teredinibacter turnerae, Hahella chejuensis, and Pseudomonas sp. ND137. C. mixtus.

In another aspect of the invention, the host cells described and/or used in any of the compositions or methods described herein are facultative anaerobic cells and progeny thereof. Facultative anaerobes can generate cellular ATP by aerobic respiration (e.g., utilization of the TCA cycle) if oxygen is present. However, facultative anaerobes can also grow in the absence of oxygen. This is in contrast to obligate anaerobes which die or grow poorly in the presence of greater amounts of oxygen. In one aspect, therefore, facultative anaerobes can serve as host cells for any of the compositions and/or methods provided herein and can be engineered to produce isoprene. Facultative anaerobic host cells can be grown under substantially oxygen-free conditions, wherein the amount of oxygen present is not harmful to the growth, maintenance, and/or fermentation of the anaerobes, or can be alternatively grown in the presence of greater amounts of oxygen.

In another aspect of the invention, the host cells described and/or used in any of the compositions or methods described herein are strict aerobic cells and progeny thereof. Strict aerobes can generate cellular ATP by aerobic respiration (e.g., utilization of the TCA cycle) if oxygen is present. However, strict aerobes die or grow poorly in the presence of greater amounts of oxygen. In one aspect, therefore, strict aerobes can serve as host cells for any of the compositions and/or methods provided herein and can be engineered to produce isoprene. Strict aerobe host cells can be alternatively grown in the presence of oxygen. In some aspects, a strict aerobe cell is a S. degradans bacterium.

The production of isoprene by the cells according to any of the compositions or methods described herein can be enhanced (e.g., enhanced by the expression of one or more heterologous nucleic acids encoding an isoprene synthase polypeptide, MVA pathway polypeptide(s), and/or a DXP pathway polypeptide(s)). As used herein, “enhanced” isoprene production refers to an increased cell productivity index (CPI) for isoprene, an increased titer of isoprene, an increased mass yield of isoprene, and/or an increased specific productivity of isoprene by the cells described by any of the compositions and methods described herein compared to cells which do not have one or more heterologous nucleic acids encoding a isoprene synthase polypeptide.

The production of isoprene by the recombinant cells described herein can be enhanced by about 5% to about 1,000,000 folds. In certain aspects, the production of isoprene can be enhanced by about 10% to about 1,000,000 folds (e.g., about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of isoprene by cells that do not express one or more heterologous nucleic acids encoding an isoprene synthase poly peptide.

In other aspects, the production of isoprene by the recombinant cells described herein can also be enhanced by at least about any of 5%, 10%, 20%, 30%, 40%, 50%, 60%, 70%, 80%, 90%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds as compared to the production of isoprene by cells that do not express one or more heterologous nucleic acids encoding isoprene synthase polypeptide.

### Vectors

Suitable vectors can be used for any of the compositions and methods described herein. For example, suitable vectors can be used to optimize the expression of one or more copies of a gene encoding a HMG-CoA reductase, an isoprene synthase, and/or one or more non-thiolase MVA pathway polypeptides. In some aspects, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, streptomycin, clindamycin, lincomycin, tetracycline, tobramycin, spectinomycin, or chloramphenicol) and/or nucleic acids that confer a metabolic advantage, such as a nutritional advantage on the host cell. In some aspects, one or more copies of HMG-CoA reductase, an isoprene synthase, and/or one or more non-thiolase MVA pathway polypeptides nucleic acid(s) integrate into the genome of host cells without a selective marker. In some embodiments, a suitable vector is a broad host range vector. In further embodiments, a suitable vector is a plasmid belonging to the incompatibility group Q (IncQ) (i.e., IncQ and IncQ-like plasmids). Examples of such a plasmid that can be used include pMMB503EH and pDSK600 (Michel L O., et al., Gene, 152(1):41-45, 1995) (Murrillo J., et al., Plasmid, 31(3):275-287, 1994). Any one of the vectors characterized or used in the Examples of the present disclosure can be used.

In one embodiment, a marine bacterium such as Saccharophagus degradans is used as a host. In this embodiment, an expression vector can be selected and/or engineered to be able to autonomously replicate in such bacterium. Promoters, a ribosome binding sequence, transcription termination sequence(s) can also be included in the expression vector, in addition to the genes listed herein. Optionally, an expression vector may contain a gene that controls promoter activity.

When a marine bacterium is used as a host, Saccharophagus degradans or the like can be used. In this case, a promoter is not particularly limited as long as it can be expressed in marine bacterium. Examples of such a promoter that can be used include the 3xlacUV5 promoter. Further, an artificially designed or modified promoter such as a tac promoter may be used. In some embodiments, the promoter is inducible. In other embodiments, the promoter is constitutive.

### Transformation Methods

Nucleic acids encoding acetoacetyl-CoA synthase, an enzyme that produces acetoacetyl-CoA synthase from malonyl-CoA and acetyl-CoA, non-thiolase MVA pathway polypeptides, DXP pathway polypeptides, isoprene synthase polypeptides, IDI, and any other enzyme needed to produce isoprene can be introduced into host cells (e.g., a plant cell, a fungal cell, a yeast cell, or a bacterial cell) by any technique known to one of the skill in the art. In some embodiments, nucleic acids encoding one or more MVA pathway polypeptide, one or more DXP pathway polypeptide and/or one or more isoprene synthase is introduced into host cells using chromosomal integration or extrachromasomal vehicles, such as plasmids.

Standard techniques for introduction of a DNA construct or vector into a host cell, such as transformation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion can be used. General transformation techniques are known in the art (See, e.g., Current Protocols in Molecular Biology (F. M. Ausubel et al. (eds.) Chapter 9, 1987; Sambrook et al., Molecular Cloning: A Laboratory Manual, 3r^{d} ed., Cold Spring Harbor, 2001; and Campbell et al., Curr. Genet. 16:53-56, 1989). The introduced nucleic acids can be integrated into chromosomal DNA or maintained as extrachromosomal replicating sequences. Transformants can be selected by any method known in the art. Suitable methods for selecting transformants are described in International Publication No. WO 2009/076676, U.S. patent application Ser. No. 12/335,071 (US Publ. No. 2009/0203102), WO 2010/003007, U.S. Patent Appl. Publ. No. 2010/0048964, WO 2009/132220, and U.S. Patent Appl. Publ. No. 2010/0003716.

A method for introduction of an expression vector is not particularly limited as long as DNA is introduced into a bacterium thereby. Examples thereof include a method using calcium ions (Cohen, S. N., et al.: Proc. Natl. Acad. Sci., USA, 69:2110-2114, 1972) and an electroporation method.

### Exemplary Cell Culture Media

As used herein, the terms “minimal medium” or “minimal media” refer to growth medium containing the minimum nutrients possible for cell growth, generally, but not always, without the presence of one or more amino acids (e.g., 1, 2, 3, 4, 5, 6, 7, 8, 9, 10, or more amino acids). Minimal medium typically contains: (1) a carbon source for bacterial growth; (2) various salts, which can vary among bacterial species and growing conditions; and (3) water. The carbon source can vary significantly, from simple sugars like glucose to more complex hydrolysates of other biomass, such as yeast extract, as discussed in more detail below. The salts generally provide essential elements such as magnesium, nitrogen, phosphorus, and sulfur to allow the cells to synthesize proteins and nucleic acids. Minimal medium can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. Medium can be supplemented with other compounds as necessary to select for desired physiological or biochemical characteristics, such as particular amino acids and the like.

Any minimal medium formulation can be used to cultivate the host cells. Exemplary minimal medium formulations include, for example, M9 minimal medium and TM3 minimal medium. Each liter of M9 minimal medium contains (1) 200 ml sterile M9 salts (64 g Na₂HPO₄-7H₂O, 15 g KH₂PO₄, 2.5 g NaCl, and 5.0 g NH₄Cl per liter); (2) 2 ml of 1 M MgSO₄ (sterile); (3) 20 ml of 20% (w/v) glucose (or other carbon source); and (4) 100 μl of 1 M CaCl₂ (sterile). Each liter of TM3 minimal medium contains (1) 13.6 g K₂HPO₄; (2) 13.6 g KH₂PO₄; (3) 2 g MgSO₄*7H₂O; (4) 2 g Citric Acid Monohydrate; (5) 0.3 g Ferric Ammonium Citrate; (6) 3.2 g (NH₄)₂SO₄; (7) 0.2 g yeast extract; and (8) 1 ml of 1000× Trace Elements solution; pH is adjusted to ˜6.8 and the solution is filter sterilized. Each liter of 1000× Trace Elements contains: (1) 40 g Citric Acid Monohydrate; (2) 30 g MnSO₄*H₂O; (3) 10 g NaCl; (4) 1 g FeSO₄*7H₂O; (4) 1 g CoCl₂*6H₂O; (5) 1 g ZnSO₄*7H₂O; (6) 100 mg CuSO₄*5H₂O; (7) 100 mg H₃BO₃; and (8) 100 mg NaMoO₄*2H₂O; pH is adjusted to ˜3.0.

An additional exemplary minimal media includes (1) potassium phosphate K₂HPO₄, (2) Magnesium Sulfate MgSO₄*7H₂O, (3) citric acid monohydrate C₆H₈O₇*H₂O, (4) ferric ammonium citrate NH₄FeC₆H₅O₇, (5) yeast extract (from biospringer), (6) 1000× Modified Trace Metal Solution, (7) sulfuric acid 50% w/v, (8) foamblast 882 (Emerald Performance Materials), and (9) Macro Salts Solution 3.36 ml All of the components are added together and dissolved in deionized H₂O and then heat sterilized. Following cooling to room temperature, the pH is adjusted to 7.0 with ammonium hydroxide (28%) and q.s. to volume. Vitamin Solution and spectinomycin are added after sterilization and pH adjustment.

A further exemplary minimal media contains per liter (1) 2.3% Instant Ocean (Aquarium Systems), (2) 0.2% yeast extract, (3) 0.5% tryptone, (4) 0.1% ammonium chloride, (5) 16.7 mM Tris-HCl, pH 8.6, and (6) 0.2% glucose, Avicel, Birchwood xylan, or citrus pectin. For plate media, marine media 2216 (BD Difco) may be used. All of the components are added together and dissolved in deionized H₂O and then heat sterilized. Streptomycin is added after sterilization.

An additional exemplary minimal media contains per liter (1) 2.3% Instant Ocean (Aquarium Systems), (2) 0.1% yeast extract, (3) 0.05% ammonium chloride, (4) 50 mM Tris-HCl, pH 7.6, and (5) 0.2% carbon source including polysaccharides (e.g., alginate, fructose, glucose, sorbitol, xylose, galactosem and lactose). For plate media, 1.5% agar may be used. All of the components, except Tris-HCl and ammonium chloride, are added together and dissolved in deionized H₂O and then heat sterilized. Tris-HCl and ammonium chloride are separately filter-sterilized then added to the cooled heat sterilized media. Streptomycin is added after sterilization. Any one of the minimal media characterized or used in the Examples of the present disclosure can be used.

Any carbon source can be used to cultivate the host cells. The term “carbon source” refers to one or more carbon-containing compounds capable of being metabolized by a host cell or organism. For example, the cell medium used to cultivate the marine bacterial cells can include any carbon source suitable for maintaining the viability or growing the marine bacterial cells. In some aspects, the carbon source is biomass (e.g., wood, a crop, waste, and a plant), a carbohydrate (such as monosaccharide, disaccharide, oligosaccharide, or polysaccharides), or invert sugar (e.g., enzymatically treated sucrose syrup). In additional aspects, the carbon source is a byproduct of biodiesel production (e.g., glycerol with high salt content). In another aspect, the carbon source is a sugar alcohol (e.g., sorbitol). In certain embodiments, wood is wood residue, trees, and shrubs. In another embodiment, waste is municipal solid waste, livestock waste, process waste, or sewage. In a further embodiment, a crop is a starch crop, sugar crop, forage crop, or an oilseed crop. In still another embodiment, a plant includes algae, water weed, marsh grass, water hyacinth, reed, and rushes.

In some aspects, the carbon source includes yeast extract or one or more components of yeast extract. In some aspects, the concentration of yeast extract is 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. In some aspects, the carbon source includes both yeast extract (or one or more components thereof) and another carbon source, such as glucose.

Exemplary polysaccharides include complex carbohydrates (e.g., agar, agarose, alginate, chitin, cellulose, fucoidan, laminarin, pectin, pullulan, starch, α-glucan, β-glucan, glucomannan, galactomannan, and xylan). Exemplary carbohydrates include C6 sugars (e.g., fructose, mannose, galactose, or glucose) and C5 sugars (e.g., xylose or arabinose). Exemplary monosaccharides include glucose, xylose and fructose; exemplary oligosaccharides include lactose and sucrose. sorbitol,

In some embodiments, any carbon source may be used as long as it can be used by a host cell such as a marine bacterium. When a marine bacterium is used as a host cell, Saccharophagus degradans or the like can be used. Examples of such a carbon source that can be used by the host cell include biomass, carbohydrates, polysaccharides, sugar alcohols, and byproduct of biodeisel production. In some embodiments, the biomass is corn cob, marsh grass, or wood. In other embodiments, the complex carbohydrates are agar, alginate, chitin, cellulose, fucoidan, laminarin, pectin, pullulan, starch α-glucan, β-glucan, glucomannan, galactomannan, or xylan. In yet another embodiment, the carbohydrates are fructose, glucose, xylose, mannose, arabinose, or lactose. In another embodiment, the sugar alcohol is sorbitol.

### Exemplary Cell Culture Conditions

Materials and methods suitable for the maintenance and growth of the recombinant marine bacterial cells of the invention are described infra, e.g., in the Examples section. Other materials and methods suitable for the maintenance and growth of bacterial cultures are well known in the art. Exemplary techniques can be found in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass. In some aspects, the cells are cultured in a culture medium under conditions permitting the expression of one or more HMG-CoA reductase, HMG-CoA synthase, isoprene synthase, DXP pathway (e.g., DXS), IDI, or lower MVA pathway polypeptides encoded by a nucleic acid inserted into the host cells.

Standard cell culture conditions can be used to culture the cells (see, for example, WO 2004/033646 and references cited therein). In some aspects, cells are grown and maintained at an appropriate temperature, gas mixture, and pH (such as at about 20° C. to about 37° C., at about 6% to about 84% CO₂, and at a pH between about 5 to about 9). In some aspects, cells are grown at 35° C. in an appropriate cell medium. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the host cells. In addition, more specific cell culture conditions can be used to culture the cells. For example, in some embodiments, the marine bacterial cells (such as S. degradans cells) express one or more heterologous nucleic acids encoding isoprene synthase under the control of an inducible promoter in a multicopy plasmid and are cultured at a temperature range between about 5° C. to 40° C. and a pH range between about pH 4.5 to about pH 10.0.

Standard culture conditions and modes of fermentation, such as batch, fed-batch, or continuous fermentation that can be used are described in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716. Batch and Fed-Batch fermentations are common and well known in the art and examples can be found in Brock, Biotechnology: A Textbook of Industrial Microbiology, Second Edition (1989) Sinauer Associates, Inc.

In some aspects, the cells are cultured under limited glucose conditions. By “limited glucose conditions” is meant that the amount of glucose that is added is less than or about 105% (such as about 100%, 90%, 80%, 70%, 60%, 50%, 40%, 30%, 20%, or 10%) of the amount of glucose that is consumed by the cells. In particular aspects, the amount of glucose that is added to the culture medium is approximately the same as the amount of glucose that is consumed by the cells during a specific period of time. In some aspects, the rate of cell growth is controlled by limiting the amount of added glucose such that the cells grow at the rate that can be supported by the amount of glucose in the cell medium. In some aspects, glucose does not accumulate during the time the cells are cultured. In various aspects, the cells are cultured under limited glucose conditions for greater than or about 1, 2, 3, 5, 10, 15, 20, 25, 30, 35, 40, 50, 60, or 70 hours. In various aspects, the cells are cultured under limited glucose conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited glucose conditions can allow more favorable regulation of the cells.

In some aspects, the bacterial cells are grown in batch culture. The bacterial cells can also be grown in fed-batch culture or in continuous culture. Additionally, the bacterial cells can be cultured in minimal medium, including, but not limited to, any of the minimal media described above. The minimal medium can be further supplemented with 1.0% (w/v) glucose, or any other six carbon sugar, or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract.

In some aspects, the recombinant cells are grown under low oxygen conditions. In other aspects, the bacterial cells are grown under atmospheric conditions comprising any of about 4%, 5%, 6%, 7%, 8%, 9%, 10%, 11%, 12%, 13%, 14%, or 15%, inclusive, including any values in between these percentages, oxygen. In other aspects, the bacterial cells are grown under atmospheric conditions comprising any of about 3-8%, 3.5-8.5%, 4-9%, 4.5-9.5%, 5-10%, 5.5-10.5%, 6-11%, or 6.5-11.5% oxygen.

### Methods of Using the Recombinant Marine Bacterial Cells to Produce Isoprene

Provided herein are methods of producing isoprene comprising culturing any of the recombinant cells described herein. In certain embodiments, the method of the present invention is a method of producing isoprene comprising a) culturing a recombinant marine bacterial cell comprising and b) producing the isoprene. In one embodiment, the recombinant marine bacterial cell is a marine saphrophytic bacterium. In another embodiment, the recombinant bacterial cell is Saccharophagus degradans 2-40. In some embodiments, the recombinant marine bacterial cell is cultured in a medium comprising biomass to produce isoprene. In other embodiments, the isoprene synthase is a plant isoprene synthase. In a further embodiment, the isoprene synthase is the P. alba synthase. In another embodiment, the isoprene synthase is an isoprene synthase variant. In certain embodiments, the recombinant bacterial cell further comprises a heterologous nucleic acid encoding one or more MVA pathway polypeptide and/or one or more DXP pathway polypeptide. The isoprene can be produced from any of the cells described herein and according to any of the methods described herein. Any of the cells can be used for the purpose of producing isoprene from carbohydrates, including six carbon sugars such as glucose, and/or biomass, including complex carbohydrates such as cellulose from plants. The cells can further comprise one or more nucleic acid molecules encoding one or more MVA pathway polypeptide(s) described above (e.g., MVK, PMK, MVD, and/or IDI). In some aspects, the cells can further comprise one or more nucleic acid molecules encoding one or more DXP pathway polypeptide(s) described above (e.g., DXS, DXR, MCT, CMK, MCS, HDS, and/or HDR). In another aspect, the cells can further comprise one or more nucleic acid molecules encoding any of the isoprene synthase polypeptide(s) described above (e.g. P. alba isoprene synthase). In some aspects, the marine bacterial cells can be any of the cells described herein. Any of the isoprene synthases or variants thereof described herein, any of the marine bacterial strains described herein, any of the promoters described herein, and/or any of the vectors described herein can also be used to produce isoprene using any of the energy sources (e.g. glucose or any other six carbon sugar and biomass) described herein. In some aspects, the method of producing isoprene further comprises a step of recovering the isoprene.

In some aspects, the amount of isoprene produced is measured at a productivity time point. In some aspects, the productivity for the cells is about any of the amounts of isoprene disclosed herein. In some aspects, the cumulative, total amount of isoprene produced is measured. In some aspects, the cumulative total productivity for the cells is about any of the amounts of isoprene disclosed herein.

In some aspects, any of the cells described herein (for examples the cells in culture) produce isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, or more nmole of isoprene/gram of cells for the wet weight of the cells/hour (nmole/g_{wcm}/hr). In some aspects, the amount of isoprene is between about 2 to about 5,000 nmole/g_{wcm}/hr, such as between about 2 to about 100 nmole/g_{wcm}/hr, about 100 to about 500 nmole/g_{wcm}/hr, about 150 to about 500 nmole/g_{wcm}/hr, about 500 to about 1,000 nmole/g_{wcm}/hr, about 1,000 to about 2,000 nmole/g_{wcm}/hr, or about 2,000 to about 5,000 nmole/g_{wcm}/hr. In some aspects, the amount of isoprene is between about 20 to about 5,000 nmole/g_{wcm}/hr, about 100 to about 5,000 nmole/g_{wcm}/hr, about 200 to about 2,000 nmole/g_{wcm}/hr, about 200 to about 1,000 nmole/g_{wcm}/hr, about 300 to about 1,000 nmole/g_{wcm}/hr, or about 400 to about 1,000 nmole/g_{wcm}/hr.

In some aspects, the cells in culture produce isoprene at greater than or about 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 100,000, or more ng of isoprene/gram of cells for the wet weight of the cells/hr (ng/g_{wcm}/h). In some aspects, the amount of isoprene is between about 2 to about 5,000 ng/g_{wcm}/h, such as between about 2 to about 100 ng/g_{wcm}/h, about 100 to about 500 ng/g_{wcm}/h, about 500 to about 1,000 ng/g_{wcm}/h, about 1,000 to about 2,000 ng/g_{wcm}/h, or about 2,000 to about 5,000 ng/g_{wcm}/h. In some aspects, the amount of isoprene is between about 20 to about 5,000 ng/g_{wcm}/h, about 100 to about 5,000 ng/g_{wcm}/h, about 200 to about 2,000 ng/g_{wcm}/h, about 200 to about 1,000 ng/g_{wcm}/h, about 300 to about 1,000 ng/g_{wcm}/h, or about 400 to about 1,000 ng/g_{wcm}/h.

In some aspects, the cells in culture produce a cumulative titer (total amount) of isoprene at greater than about any of or about any of 1, 10, 25, 50, 100, 150, 200, 250, 300, 400, 500, 600, 700, 800, 900, 1,000, 1,250, 1,500, 1,750, 2,000, 2,500, 3,000, 4,000, 5,000, 10,000, 50,000, 100,000, or more mg of isoprene/L of broth (mg/L_{broth}, wherein the volume of broth includes the volume of the cells and the cell medium). In some aspects, the amount of isoprene is between about 2 to about 5,000 mg/L_{broth}, such as between about 2 to about 100 mg/L_{broth}, about 100 to about 500 mg/L_{broth}, about 500 to about 1,000 mg/L_{broth}, about 1,000 to about 2,000 mg/L_{broth}, or about 2,000 to about 5,000 mg/L_{broth}. In some aspects, the amount of isoprene is between about 20 to about 5,000 mg/L_{broth}, about 100 to about 5,000 mg/L_{broth}, about 200 to about 2,000 mg/L_{broth}, about 200 to about 1,000 mg/L_{broth}, about 300 to about 1,000 mg/L_{broth}, or about 400 to about 1,000 mg/L_{broth}.

In some aspects, the isoprene produced by the cells in culture (such as any of the recombinant cells described herein) comprises at least about 1, 2, 5, 10, 15, 20, or 25% by volume of the fermentation offgas. In some aspects, the isoprene comprises between about 1 to about 25% by volume of the offgas, such as between about 5 to about 15%, about 15 to about 25%, about 10 to about 20%, or about 1 to about 10%.

### Exemplary Purification Methods

In some aspects, any of the methods described herein further include a step of recovering isoprene produced by any of the recombinant cells disclosed herein. In some aspects, the isoprene is recovered by absorption stripping (See, e.g., U.S. Patent Appl. Publ. No. 2011/0178261). Suitable purification methods are described in more detail in U.S. Patent Application Publication No. US2010/0196977 A1.

Throughout this specification, various patents, patent applications and other types of publications (e.g., journal articles) are referenced. The disclosure of all patents, patent applications, and publications cited herein are hereby incorporated by reference in their entirety for all purposes.

The invention can be further understood by reference to the following examples, which are provided by way of illustration and are not meant to be limiting.

### EXAMPLES

The biomass-degrading bacterium Saccharophagus degradans was engineered to directly make isoprene. The gene ipsS was introduced into and expressed in S. degradans to make a consolidated bioprocessing organism that produces isoprene directly from biomass.

### Example 1

### Cloning of the Gene Encoding Isoprene Synthase to an Expression System Compatible with S. degradans

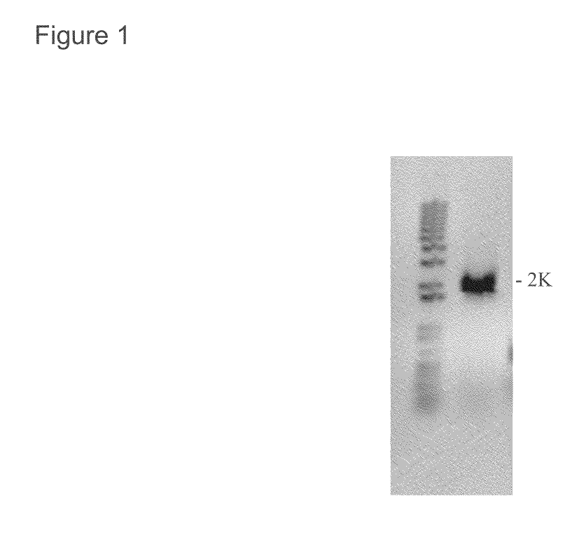

The P. alba gene encoding isoprene synthase (ispS) enzyme was PCR amplified from pET24a-ispS using a Forward primer (5′-ATAGCGAATTCAGAAGGAGATATACCATGGAAGCACGTCGCTCTGCGAACT-3′) (SEQ ID NO:11) containing an EcoRI restriction enzyme recognition site (underlined), a Reverse primer (5′-ATACGCGGATCCTTAGCGTTCAAACGGCAGAATCGGT-3′) (SEQ ID NO:12) containing a BamHI restriction enzyme recognition site (underlined), and the Phusion High-Fidelity DNA Polymerase (New England Biolabs) according to the manufacturer's instructions (FIG. 1). After purification of the PCR product, the fragment was digested with EcoRI and BamHI, and ligated with EcoRI/BamHI-digested pMMB503H (provided by Michael Bagdasarian), a derivative of pMMB67EH which confers streptomycin resistance (Fiirste, J. P., W. Pansegrau, et al. (1986). “Molecular cloning of the plasmid RP4 primase region in a multi-host-range tacP expression vector.” Gene 48: 119-131) to form plasmid pMMB503EH-ispS. After ligation overnight at 16° C., the ligation mixture was electroporated into DHSa E. coli cells (Invitrogen). The electroporated cells were incubated for 1 hour at 37° C. and spread onto Luria-Bertani plates containing streptomycin. Colony PCR was carried out the next day to identify successful transformants that were subsequently picked and grown overnight in broth culture containing streptomycin for subsequent isolation of pMMB503EH-ispS. Insertion of the P. alba ispS gene into pMMB503EH was confirmed by sequencing. The pET24a-IspS provided herein is described U.S. Patent Application Publication No.: 2010/0003716, the contents of which are expressly incorporated herein by reference in their entirety with respect to the isoprene synthases and isoprene synthase variants.

### Example 2

### Expression of the ispS Gene in E. coli

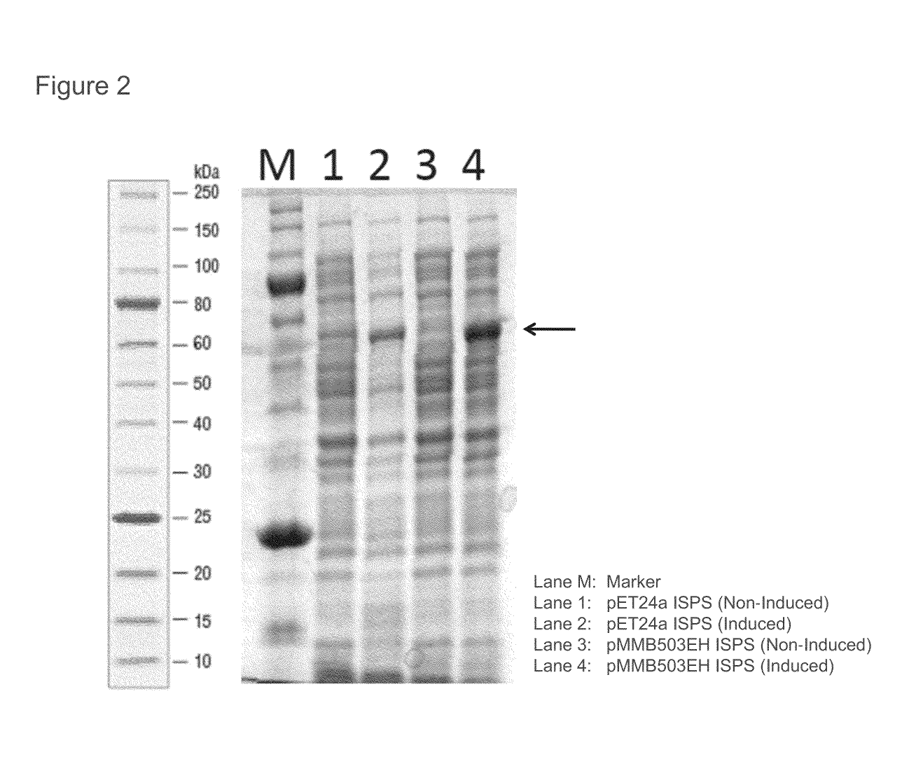

The pET24a-ispS and pMMB503EH-ispS plasmids were each electroporated into Rosetta2™ (DE3) (Novagen) for subsequent isoprene synthase protein expression analysis. Briefly, Rosetta transformants were picked and cells transformed with pMMB503EH-ispS were grown at 37° C. to an OD600 of 0.6 in Luria-Bertani broth culture supplemented with streptomycin and chloramphenicol. Cells transformed with pET24a-ispS were grown at 37° C. to an OD₆₀₀ of 0.6 in Luria-Bertani broth culture supplemented with kanamycin. Cultures were either induced by the addition of 0.4 mM IPTG (isopropyl-β-D-thiogalactopyranoside) or non-induced (no IPTG) and maintained at 16° C. with shaking for 16 hours before cells were harvested and lysed. Crude lysates of non-induced (no IPTG) and induced (with IPTG) cells were analyzed by sulfate-polyacrylamide gel electrophoresis (FIG. 2). The presence of a band corresponding to isoprene synthase protein at a molecular weight of about 60 kDa in crude lysates from the induced cultures demonstrates the P. alba ispS gene is expressed from the inducible pET24a-ispS and pMMB503EH-ispS plasmids in E. coli.

Rosetta transformants harboring pMMB503EH-ipsS or pET24a-ipsS were grown as described above. An overnight culture in 50 ml broth culture in LB medium was induced by the addition of IPTG and after 16 hrs subject to the “sniff” test. Cultures were scored for the presence of a refinery-like smell, which is indicative of isoprene production. Induced Rosetta transformants harboring pMMB503EH-ipsS or pET24a-ipsS emitted a refinery-like smell as compared to non-induced Rosetta transformants that lacked the refinery-like smell.

### Example 3

### Expression of the ispS Gene in S. degradans 2-40

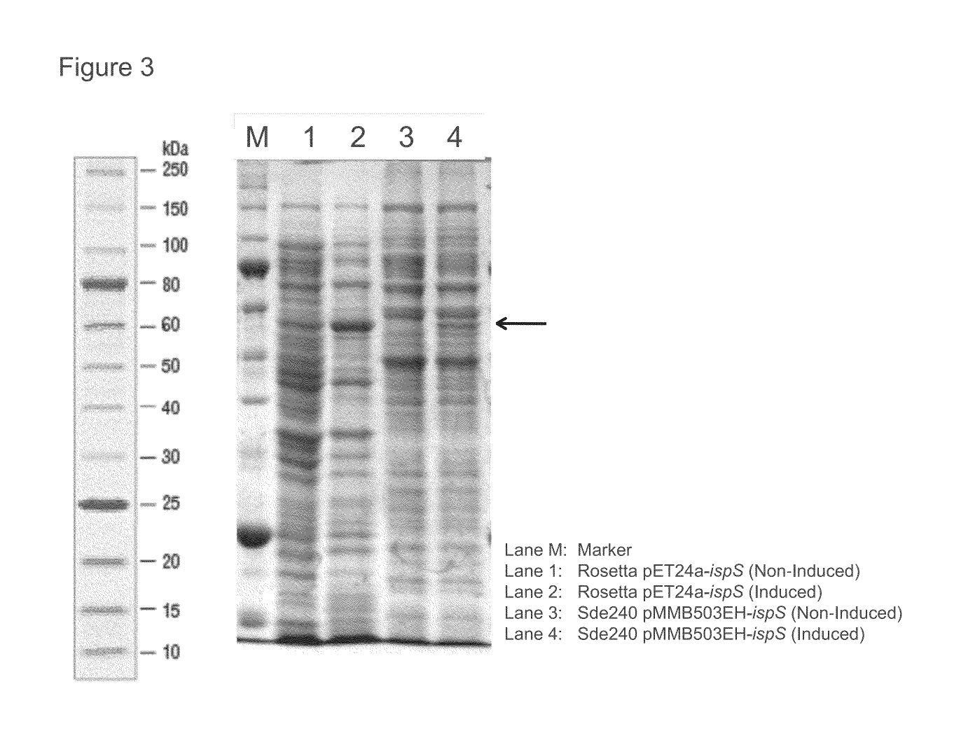

The pET24a-ispS and pMMB503EH-ispS plasmids were each isolated from Rosetta2 (D3) cells and electroporated into Saccharophagus degradans 2-40 (Sde240) for subsequent isoprene synthase protein expression analysis. Sde240 transformants were grown in media containing 2.3% (w/v) Instant Ocean (Aquarium Systems, Mentor, Ohio), 0.05% Yeast Extract, 0.5% ammonium chloride, and 16.7 mM Tris pH 8.6 supplemented with 0.2% carbon source and streptomycin to an OD₆₀₀ of 0.3 at 30° C. Cultures were either induced by the addition of 1 mM IPTG (isopropyl-β-D-thiogalactopyranoside) or non-induced (no IPTG) for 6 hours before cells were harvested. Crude cellular lysates were prepared using a BeadBeater (Biospec) and analyzed by sulfate-polyacrylamide gel electrophoresis (FIG. 3). The presence of a band corresponding to isoprene synthase protein at a molecular weight of about 60 kDa in crude lysates from the induced cultures demonstrates the P. alba ispS gene is expressed from inducible pET24a-ispS in Rosetta cells and from inducible pMMB503EH-ispS in Sde240 cells.

S. degradans transformants harboring pMMB503EH-ipsS or pET24a-ipsS were grown as described above. An overnight culture in 50 ml broth culture in Sde2-40 medium was induced by the addition of IPTG and after 6 hrs subject to the “sniff” test. Cultures were scored for the presence of a refinery-like smell, which is indicative of isoprene production. Induced S. degradans transformants harboring pMMB503EH-ipsS or pET24a-ipsS emitted a refinery-like smell as compared to non-induced S. degradans transformants that lacked the refinery-like smell.

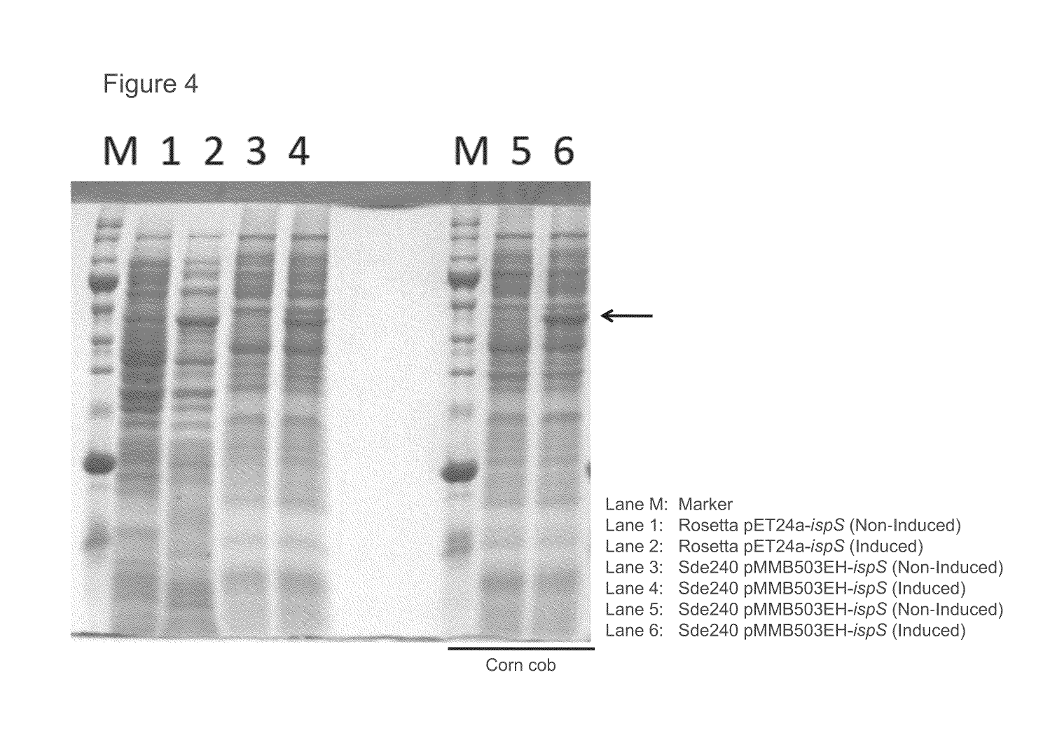

Cultures of Sde240 cells harboring the pMMB503EH-ispS plasmid were grown on milled corn cob and assayed for isoprene synthase expression as described above. Cultures were either induced by the addition of 1 mM IPTG (isopropyl-β-D-thiogalactopyranoside) or non-induced (no IPTG) for 6 hours before cells were harvested. Crude cellular lysates were prepared using a BeadBeater (Biospec) and analyzed by sulfate-polyacrylamide gel electrophoresis (FIG. 4). The presence of a band corresponding to isoprene synthase protein at a molecular weight of about 60 kDa in crude lysates from the induced cultures demonstrates the P. alba ispS gene is expressed from the inducible pMMB503EH-ispS plasmid in Sde240 cells grown on corn cob as the major carbon source.

### Example 4

### Expression of the Isoprene Synthase in S. degradans and E. coli

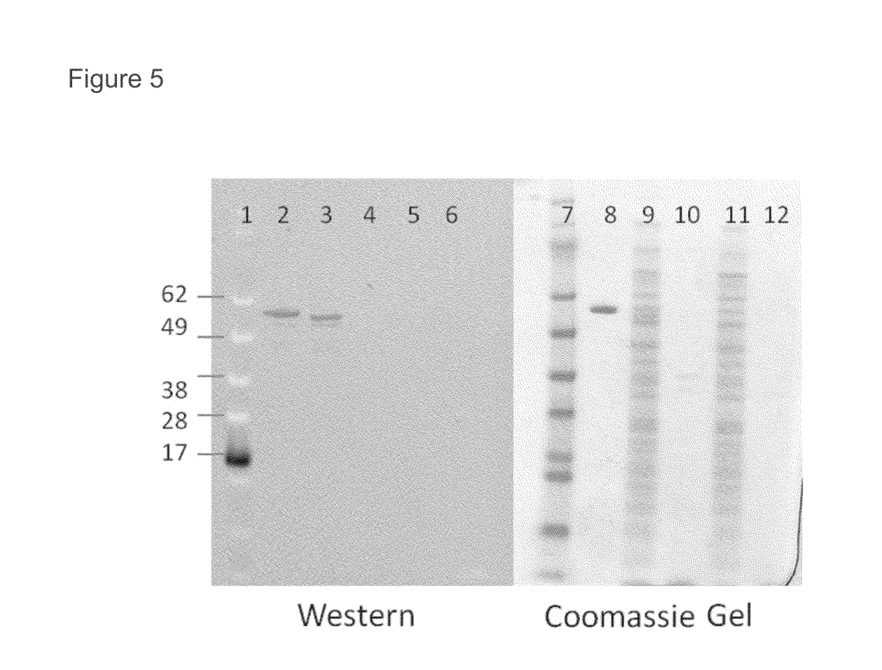

Cultures of Sde240 cells harboring the pMMB503EH-ispS plasmid that were either induced with IPTG or non-induced were harvested by centrifugation and resuspended in 5 mL buffer containing 100 mM Tris, 100 mM NaCl pH 7.6 and 0.1 mg/mL DNAse I. Cells were lysed by French press and 1 mL of the lysate was centrifuged at 14,000×g in a microfuge at 4° C. for 15 minutes. The pellet was resuspended in 1 mL buffer containing 100 mM Tris and 100 mM NaCl, pH 7.6 for analysis by SDS-PAGE using 4-12% Bis-Tris NUPAGE Gels. The pellet and supernatant fractions were quantified for proteins levels using the BioRad Protein Assay according to the manufacturer's instructions. Isoprene synthase expression was analyzed by Coomasie gel staining (FIG. 5, right panel) and western blot analysis (FIG. 5, left panel). Isoprene synthase was detected for western blot analysis by probing with rabbit anti-IspS primary antibody and goat anti-rabbit Alexa Fluor 488 secondary antibody. Protein analysis demonstrated that a majority of the P. alba isoprene synthase expressed in S. degradans was contained in the soluble fraction (FIG. 5, Lane 2 and 8).

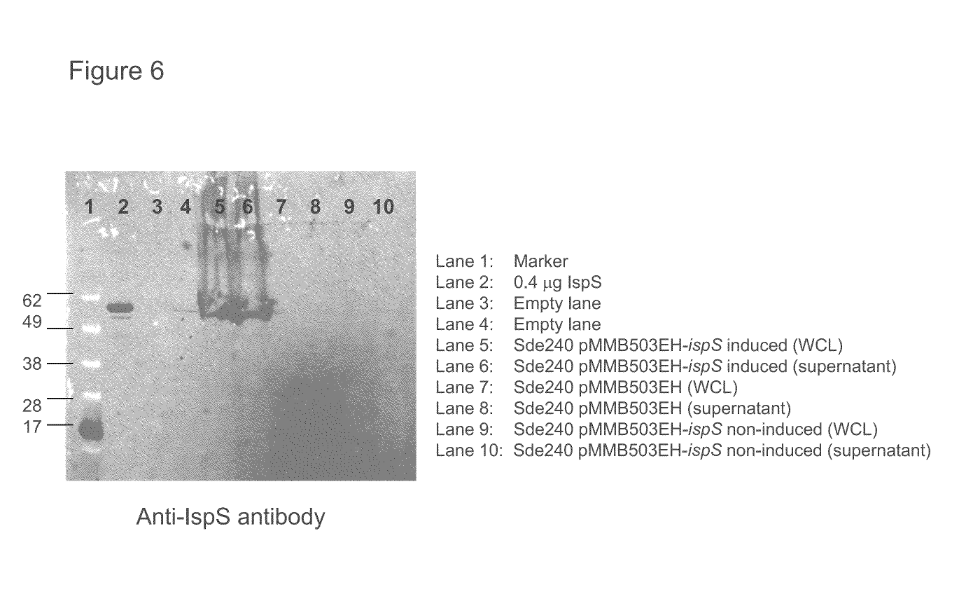

Cultures of Sde240 cells harboring the pMMB503EH-ispS plasmid that were either induced with IPTG or non-induced were harvested by centrifugation and resuspended in 5 mL buffer containing 100 mM Tris, 100 mM NaCl pH 7.6 and 0.1 mg/mL DNAse I. Cells were lysed by French press and 1 mL of the lysate was centrifuged at 14,000×g in a microfuge at 4° C. for 15 minutes. Sample of the supernatant and of the whole cell lysate (WCL) was loaded onto 4-12% Bis-Tris NUPAGE Gels in a 15 μl volume for electrophoresis. Isoprene synthase expression was analyzed by western blot analysis (FIG. 6). Isoprene synthase was detected for western blot analysis by probing with rabbit anti-IspS primary antibody and goat anti-rabbit Alexa Fluor 488 secondary antibody. Protein analysis demonstrated that soluble isoprene synthase was expressed in S. degradans that was induced with IPTG (FIG. 6, Lane 5 and 6) at about the same molecular weight of purified isoprene synthase (FIG. 6, Lane 2). No isoprene synthase was produced by non-induced S. degradans harboring the pMMB503EH-ispS plasmid (FIG. 6, Lane 9 and 10). Similarly, no isoprene synthase was produced by S. degradans cultures transformed with empty pMMB503EH vector (FIG. 6, Lane 7 and 8).

### Example 5

### Production of Isoprene by S. degradans Expressing P. alba Isoprene Synthase

The specific productivity of isoprene from the engineered S. degradans 2-40 strain was determined. To induce expression of the genes encoded by the plasmid, Sde240 cultures were grown in media containing 2.3% (w/v) Instant Ocean (Aquarium Systems, Mentor, Ohio), 0.05% Yeast Extract, 0.5% ammonium chloride, and 16.7 mM Tris pH 8.6 supplemented with 0.2% carbon source and streptomycin to an OD₆₀₀ of 0.3 at 30° C. Cultures were either induced by the addition of 1 mM IPTG (isopropyl-β-D-thiogalactopyranoside) or non-induced (no IPTG) for 6 hours before cells were harvested by centrifugation and resuspended in 5 mL buffer containing 100 mM Tris, 100 mM NaCl pH 7.6 and 0.1 mg/mL DNAse I. Cells were lysed by French press and 1 mL of the lysate was centrifuged at 14,000×g in a microfuge at 4° C. for 15 minutes. The supernatant was assayed for isoprene synthase activity at 34° C. for 15 min in a 100 μL reaction containing 25 μL lysate, 5 mM DMAPP, 50 mM MgCl₂ and 65 μL of buffer containing 100 mM Tris, 100 mM NaCl, pH 7.6. The reaction was terminated by addition of 100 μL of 250 mM EDTA and the levels of isoprene in the headspace were determined by flame ionization detector coupled to a gas chromatograph also known as GC-MS (Model G1562A, Agilent Technologies) (Mergen et al., LC GC North America, 28(7):540-543, 2010). The induced sample contained 819 μg/L isoprene as compared to the non-induced sample that containing 8.44 μg/L Isoprene.

### Examples 6

### Production of Isoprene by S. degradans Expressing P. alba Isoprene Synthase Utilizing Biomass as a Carbon Source

Saccharophagus degradans 2-40 expressing pMMB503EH-ispS is grown in Sde2-40.2 medium (Yeast Extract, 0.5 g/L; Ammonium chloride, 5 g/L; 50 mM Trizma-HCl pH 7.6, Instant Sea Salts, 23 g/L)+2 g/L glucose and 50 mg/L streptomycin), at 30° C. with shaking at 200 rpm. The day after incubation, 200 μl of a culture OD₆₀₀ of 0.5 is inoculated into 20 mL of the Sde2-40.2 medium containing no carbon source, or with 10 g/L glucose, or with 200 mg of hardwood pulp, corn fiber, acid pretreated bagasse or acid pretreated corn stover that is added to the medium before heat sterilization. The media is supplemented with 50 mg/L streptomycin. Inoculated cell cultures are incubated at 30° C. with shaking at 200 rpm. After a 2 hour incubation for the cultures containing media with no carbon source or with 10 g/L glucose, and after a 4 hour incubation for the cultures containing hardwood pulp, corn fiber, acid pretreated bagasse or acid pretreated corn stover as a carbon source, a total of 400 mM IPTG is added to the culture to induce expression of the isoprene synthase. Growth of the cells is measured on a spectrophotometer (OD 600 nm) for the cultures with no carbon source or for cultures supplemented with glucose. For the cultures containing hardwood pulp, corn fiber, acid pretreated bagasse or acid pretreated corn stover, colony forming unit (cfu) are measured according to a method similar to what is described in Gerhardt et al., 1994, Methods for General and Molecular Bacteriology, American Society for Microbiology. Off-gas analysis of isoprene produced by the recombinant bacteria is performed using a gas chromatograph-mass spectrometer (GC-MS) (Agilent) headspace assay. A 100 μl sample of whole broth is placed in a sealed GC vial and incubated at 34° C. with shaking at 200 rpm for a fixed time of 60 minutes or 4 hours. Following a heat kill step, consisting of incubation at 70° C. for 7 minutes, the sample is loaded on the GC. The reported specific productivity is the amount of isoprene in μg/L read by the GC divided by the incubation time and the measured OD₆₀₀ or cfu.

What is claimed is:

1. A recombinant marine bacterial cell capable of increased production of isoprene, the cell comprising one or more copies of a heterologous polynucleotide sequence encoding an isoprene synthase, wherein said cell produces isoprene at a higher level than isoprene produced by a cell that does not comprise one or more copies of a heterologous polynucleotide sequence encoding an isoprene synthase.

2. The recombinant marine bacterial cell of claim 1, wherein the cell is a gram-positive bacterium or a gram-negative bacterium.

3. The recombinant marine bacterial cell of claim 1, wherein the cell is a cellulolytic bacterium, an agarolytic bacterium, an alginolytic bacterium, a glucanolytic bacterium, a chitinolytic bacterium, a pectinolytic bacterium, a xylanolytic bacterium or a mannanolytic bacterium.

4. The recombinant marine bacterial cell of claim 1, wherein the cell is a marine γ-proteobacterium, a marine saprophytic bacterium, a Microbulbifer, a Marinobacterium or a Saccharophagus.

5. The recombinant marine bacterial cell of claim 4, wherein the cell is selected from the group consisting of Saccharophagus degradans 2-40, Microbulbifer hydrolyticus IRE-31 and Marinobacterium georgiense KW-40.

6. The recombinant marine bacterial cell of claim 5, wherein the cell is Saccharophagus degradans 2-40 having the identifying characteristics of ATCC 43961.

7. The recombinant marine bacterial cell of claim 1, wherein the cell is cultured in a medium comprising a carbon source selected from the group consisting of biomass, carbohydrates, sugar alcohols, and byproducts of biodiesel production.

8. The recombinant marine bacterial cell of claim 7, wherein the biomass is selected from the group consisting of wood, crops, waste, and plants.

9. The recombinant marine bacterial cell of claim 7, wherein the carbohydrates are selected from the group consisting of monosaccharides, disaccharides, oligosaccharides, and polysaccharides.

10. The recombinant marine bacterial cell of claim 7, wherein the carbohydrates are selected from the group consisting of agar, agarose, alginate, chitin, cellulose, fucoidan, laminarin, pectin, pullulan, starch α-glucan, β-glucan, glucomannan, galactomannan, and xylan.

11. The recombinant marine bacterial cell of claim 1, wherein the cell is cultured in a medium comprising a carbon source selected from the group consisting of glucose, glycerol, glycerine, dihydroxyacetone, yeast extract, biomass, molasses, sucrose, corn cob, algae, cellulose, xylan, pectin, agar, alginate, chitin, α-glucans, β-glucans, laminarin, glucomannan, galactomannan, march grass, and oil.

12. The recombinant marine bacterial cell of claim 1, wherein the isoprene synthase is a plant isoprene synthase.

13. The recombinant marine bacterial cell of claim 12, wherein the plant isoprene synthase is a poplar isoprene synthase, a kudzu isoprene synthase, a willow isoprene synthase, or a eucalyptus isoprene synthase.

14. The recombinant marine bacterial cell of claim 12, wherein the plant isoprene synthase is an isoprene synthase from Pueraria or Populus or a hybrid, Populus alba×Populus tremula.

15. The recombinant marine bacterial cell of claim 14, wherein the isoprene synthase is selected from the group consisting of Pueraria montana or Pueraria lobata, Populus tremuloides, Populus alba, Populus nigra, and Populus trichocarpa.

16. The recombinant marine bacterial cell of claim 1, wherein the isoprene synthase is the P. alba isoprene synthase of SEQ ID NO: 1.

17. The recombinant marine bacterial cell of claim 1, wherein the isoprene synthase is an isoprene synthase variant.

18. The recombinant marine bacterial cell of claim 1, wherein the cell further comprises a heterologous polynucleotide sequence encoding for one or more MVA pathway polypeptide and/or one or more DXP pathway polypeptide.

19. The recombinant marine bacterial cell of claim 18, wherein the cell further comprises a heterologous polynucleotide sequence encoding for one or more IDI polypeptide.

20. The recombinant marine bacterial cell of claim 1, wherein any one or more copies of a heterologous polynucleotide sequence is overexpressed.

21. The recombinant marine bacterial cell of claim 1, wherein the heterologous polynucleotide sequence is cloned into a multicopy plasmid.

22. The recombinant marine bacterial cell of claim 1, wherein the heterologous polynucleotide sequence is cloned into an IncQ or IncQ-like plasmid.

23. The recombinant marine bacterial cell of claim 1, wherein the heterologous polynucleotide sequence is placed under an inducible promoter or a constitutive promoter.

24. The recombinant marine bacterial cell of claim 1, wherein any one or more of the heterologous polynucleotide sequences is integrated into the chromosome of the bacterial cell.

25. A method of producing isoprene, comprising:
a) culturing a recombinant marine bacterial cell comprising one or more copies of a heterologous polynucleotide sequence encoding an isoprene synthase in under suitable culture conditions for production of isoprene, wherein said cell produces isoprene at a higher level than isoprene produced by a cell that does not comprise one or more copies of a heterologous sequence encoding an isoprene synthase; and
b) producing the isoprene.

26. The method of claim 25, further comprising (c) recovering the isoprene.

27. The method of claim 26, further comprising (d) polymerizing the isoprene.

28. The method of claim 25, wherein the cell is a gram-positive bacterium or a gram-negative bacterium.

29. The method of claim 25, wherein the cell is a cellulolytic bacterium, an agarolytic bacterium, an alginolytic bacterium, a glucanolytic bacterium, a chitinolytic bacterium, a pectinolytic bacterium, a xylanolytic bacterium or a mannanolytic bacterium.

30. The method of claim 25, wherein the cell is a marine γ-proteobacterium, a marine saprophytic bacterium, a Microbulbifer, a Marinobacterium or a Saccharophagus.

31. The method of claim 30, wherein the cell is selected from the group consisting of Saccharophagus degradans 2-41, Microbulbifer hydrolyticus IRE-31 and Marinobacterium georgiense KW-40.

32. The method of claim 31, wherein the cell is Saccharophagus degradans 2-40 having the identifying characteristics of ATCC 43961.

33. The method of claim 25, wherein the cell is cultured in a medium comprising a carbon source selected from the group consisting of biomass, carbohydrates, sugar alcohols, and byproducts of biodiesel production.

34. The method of claim 33, wherein the biomass is selected from the group consisting of wood, crops, waste, and plants.

35. The method of claim 33, wherein the carbohydrates are selected from the group consisting of monosaccharides, disaccharides, oligosaccharides, and polysaccharides.

36. The method of claim 33, wherein the carbohydrates are selected from the group consisting of agar, agarose, alginate, chitin, cellulose, fucoidan, laminarin, pectin, pullulan, starch α-glucan, β-glucan, glucomannan, galactomannan, and xylan.

37. The method of claim 25, wherein the cell is cultured in a medium comprising a carbon source selected from the group consisting of glucose, glycerol, glycerine, dihydroxyacetone, yeast extract, biomass, molasses, sucrose, corn cob, algae, cellulose, xylan, pectin, agar, alginate, chitin, α-glucans, β-glucans, laminarin, glucomannan, galactomannan, march grass, and oil.

38. The method of claim 25, wherein the isoprene synthase is a plant isoprene synthase.

39. The method of claim 38, wherein the plant isoprene synthase is a poplar isoprene synthase, a kudzu isoprene synthase, a willow isoprene synthase, or a eucalyptus isoprene synthase.

40. The method of claim 38, wherein the plant isoprene synthase is an isoprene synthase from Pueraria or Populus or a hybrid, Populus alba×Populus tremula.

41. The method of claim 40, wherein the isoprene synthase is selected from the group consisting of Pueraria montana or Pueraria lobata, Populus tremuloides, Populus alba, Populus nigra, and Populus trichocarpa.

42. The method of claim 25, wherein the isoprene synthase is the P. alba isoprene synthase of SEQ ID NO: 1.

43. The method of claim 25, wherein the isoprene synthase is an isoprene synthase variant.

44. The method of claim 25, wherein the cell further comprises a heterologous polynucleotide sequence encoding for one or more MVA pathway polypeptide and/or one or more DXP pathway polypeptide.

45. The method of claim 44, wherein the cell further comprises a heterologous polynucleotide sequence encoding for one or more IDI polypeptide.

46. The method of claim 25, wherein any one or more copies of a heterologous polynucleotide sequence is overexpressed.

47. The method of claim 25, wherein the heterologous polynucleotide sequence is cloned into a multicopy plasmid.

48. The method of claim 25, wherein the heterologous polynucleotide sequence is cloned into an IncQ or IncQ-like plasmid.

49. The method of claim 25, wherein the heterologous polynucleotide sequence is placed under an inducible promoter or a constitutive promoter.

50. The method of claim 25, wherein any one or more of the heterologous polynucleotide sequences is integrated into the chromosome of the bacterial cell.

51. A composition comprising isoprene produced by the recombinant marine bacterial cell of claim 1.
